(19) United States
(12) Patent Application Publication
(10) Pub. No.: US 2018/0215805 A1
(43) Pub. Date: Aug. 2, 2018

(54) THERAPEUTIC AND DIAGNOSTIC AGENTS

(71) Applicant: NEXVET AUSTRALIA PTY LTD, Melbourne, Victoria (AU)

(72) Inventors: Kathryn HJERRILD, Corvallis, OR (US); David GEARING, Camberwell (AU)

(21) Appl. No.: 15/546,889

(22) PCT Filed: Jan. 29, 2016
(86) PCT No.: PCT/AU2016/050052

§ 371 Date: Jul. 27, 2017

(30) Foreign Application Priority Data
Jan. 29, 2015 (AU) 2015900260

(51) Int. Cl. C07K 14/715

(52) U.S. Cl. CPC C07K 14/7151; A61K 38/00; C07K 2319/70; C07K 2319/30

(57) ABSTRACT

Provided herein is an isolated tumour necrosis factor receptor (TNFR) polypeptide capable of binding to TNF, or a TNF-binding fragment thereof, wherein the polypeptide comprises an amino acid sequence of SEQ ID NO:1, or an amino acid sequence that has at least 60% identity thereto and wherein the polypeptide or TNF-binding fragment comprises an N-terminal VPAQV motif (SEQ ID NO: 68), with the proviso that the polypeptide is not mouse TNFR p80 isoform. Also provided is a fusion protein comprising said polypeptide, or a TNF-binding fragment thereof, linked to a fusion partner, such as the CH2 and CH3 domains of an immunoglobulin heavy chain constant region. The polypeptides and fusion proteins disclosed herein may be used for diagnostic purposes and in the treatment or prevention of conditions mediated by TNF, particularly in non-human animals such as feline and equine.

### FIELD

The present invention relates generally to agents useful in the treatment, prevention and diagnosis of conditions associated with tumour necrosis factor (TNF) in non-human animals.

### BACKGROUND

Companion, athletic and work animals, such as cats, dogs and horses, develop inflammatory diseases similar to those that occur in humans, examples of which include plasmatic-lymphocytic synovitis, systemic lupus erythematosus (SLE), vasculitis and a variety of autoimmune skin diseases. Traumatically-induced diseases of joints are another relatively common affliction, particularly in horses, ranging from a mild “sprain” to a severe catastrophic injury with complete loss of support for the joints, often associated with inflammation. For race horses in particular, this can often mean that the animal needs to be put down at substantial economic and personal loss.

Arthritis (e.g., rheumatoid arthritis (RA), osteoarthritis, immune-mediated polyarthritis), often referred to as degenerative joint disease (DJD), is another inflammatory condition that afflicts companion and athletic animals. Typically associated with pain, arthritis makes it difficult for animals to maintain mobility. The condition is normally characterized as a slowly progressive chronic disease of the joint in which joint cartilage wears away. Despite its severity and impact on quality of life, where treatment is proposed, current regimes for arthritis, particularly in animals, are merely designed to manage the disease and reduce symptoms such as pain, stiffness and immobility. For instance, depending on the severity of the arthritic condition, a veterinarian may prescribe the use of non-steroidal anti-inflammatory agents or an oral or injectable joint supplement, such as hyaluronic acid or glucosamine. The direct injection into the affected joint with a corticosteroid and hyaluronic acid has also been prescribed in some instances. Whilst studies have reported that DJD is common in domesticated animals such as cats, relatively little is known about the direct relationship between DJD, pain and mobility impairment in companion animals.

The use of tumour necrosis factor (TNF) antagonists, such as anti-TNF antibodies, soluble TNF receptor proteins and TNF receptor-Fc fusion proteins has shown that the pro-inflammatory effects mediated by TNF can be mitigated in humans. For example, recombinant TNFR-Fc fusion proteins such as ENBREL™ (etanercept, Immunex) have shown that TNF antagonism can be used to treat human rheumatoid arthritis.

Whilst the use of ENBREL™ as a therapeutic agent for the treatment of inflammatory conditions has shown promise in humans, its use for the treatment of inflammatory conditions in non-human animals, such as dogs, is not indicated beyond modelling of human disease. This is attributed, at least in part, to immunogenicity that develops in a non-human species when administered with a heterologous protein derived from another species, such as ENBREL™.

There is a need to treat inflammatory conditions in non-human animals as well as other conditions exacerbated by or otherwise associated with TNF-mediated signalling.

### SUMMARY

The present disclosure is predicated, in part, on the identification of the correct amino acid sequences of the extracellular domain of equine and feline TNFR p80 isoforms. Previously published amino acid sequences did not identify this domain, resulting in an inability to exploit animal TNFR products. In accordance with the present invention, TNFR p80 equine and feline isoforms, including TNF-binding fragments thereof, and fusion proteins comprising any of the foregoing, are used as therapeutic or prophylactic agents to bind to and sequester, inhibit, abrogate, antagonize or otherwise block the biological activity of TNF in an equine or feline subject, while minimising or avoiding the immunogenicity that is typically associated with the administration of existing, heterologous agents to an equine or feline subject. These agents also have use in the development of diagnostic assays for conditions mediated, at least in part, by TNF.

With respect to equine TNFR p80, the present inventors have surprisingly found that the predicted equine p80 TNFR sequence (TNFRSF1B), as represented by Genbank Accession No. XM_005607617 (SEQ ID NO:19), has an incorrect leader sequence (MGEEAGVEGARASPFYSYLLHVEKSPITFPLQ; SEQ ID NO:18) derived from an incorrectly assigned exon 1 in the genome sequence. Furthermore, the present inventors have surprisingly found that the incorrect leader sequence shown for the predicted equine TNFR p80 amino acid sequence does not conform to the consensus and is not recognized as a signal peptide. A molecule lacking a signal peptide, as is the case for XM_005607617, would not be secreted from the cell and would thus be non-functional. Accordingly, the predicted equine TNFR p80 isoform (XM_005607617) is incapable of being expressed in a mammalian cell, by virtue of its lack of signal sequence.

Similarly, the present inventors have surprisingly found that the predicted feline p80 TNFR sequences, as represented, for example, by Genbank Accession No. XP_003989632.1 (SEQ ID NO:64), also has an incorrect leader sequence (MSNCGHVLALSGVPPGWVWGCS; SEQ ID NO:65) derived from an incorrectly assigned exon 1 in the genome sequence. The incorrect leader sequence shown for XP_003989632.1 does not conform to the consensus and is not recognized as a signal peptide. A molecule lacking a signal peptide, as is the case for XP_003989632.1, would not be secreted from the cell and would thus be non-functional. Thus, the predicted feline TNFR p80 isoform (XP_003989632.1) is incapable of being expressed in a mammalian cell, by virtue of its lack of signal sequence.

By using a degenerate primer designed on the DNA sequences that encode the N-terminal sequences of human, mouse, pig and canine TNFR p80 isoforms, the present inventors have correctly identified the N-terminal sequence for equine and feline TNFR p80 isoforms. A signal peptide is predicted for the translated amino acid sequence, with cleavage site at analogous positions. Cleavage of the signal sequence gives rise to mature TNFR p80 polypeptides comprising an N-terminal VPAQV motif.

Thus, enabled herein is an isolated tumor necrosis factor receptor (TNFR) polypeptide capable of binding to TNF, or a TNF-binding fragment of the polypeptide, wherein the polypeptide comprises an amino acid sequence of SEQ ID NO:1, or an amino acid sequence that has at least 60% similarity thereto after optimal alignment, and wherein the polypeptide or the TNF-binding fragment thereof comprises an N-terminal VPAQV motif, with the proviso that the polypeptide is not a mouse TNFR p80. In an embodiment, the polypeptide is an equine TNFR p80 comprising an N-terminal VPAQVVF motif. In another embodiment, the polypeptide is a feline TNFR p80 comprising an N-terminal VPAQVAL motif.

In an embodiment, taught herein is an isolated tumor necrosis factor receptor (TNFR) polypeptide capable of binding to TNF, or a TNF-binding fragment of the polypeptide, wherein the polypeptide comprises an amino acid sequence of SEQ ID NO:1, or an amino acid sequence that has at least 60% identity thereto after optimal alignment, and wherein the polypeptide or the TNF-binding fragment thereof comprises an N-terminal VPAQV motif, with the proviso that the polypeptide is not a mouse TNFR p80. In an embodiment, the polypeptide is an equine TNFR p80 comprising an N-terminal VPAQVVF motif. In another embodiment, the polypeptide is a feline TNFR p80 comprising an N-terminal VPAQVAL motif.

Enabled herein is an isolated polynucleotide encoding the polypeptides or TNF-binding fragments, as herein described. In an embodiment, there is provided an isolated polynucleotide comprising the nucleic acid sequence of SEQ ID NO:6.

Taught herein is a fusion protein comprising the polypeptide, or TNF-binding fragments, as herein described, linked to a fusion partner.

The present specification is also instructional for an isolated polynucleotide encoding the fusion proteins, as herein described.

Disclosed herein is a pharmaceutical composition comprising any of the polypeptides, TNF-binding fragments or fusion proteins, as herein described.

Provided herein are also any of the polypeptides, TNF-binding fragments or fusion proteins, as herein described, for use in the treatment or prevention of a condition mediated by TNF in an equine.

Also provided herein are any of the polypeptides, TNF-binding fragments or fusion proteins, as herein described, for use in the treatment or prevention of a condition mediated by TNF in a feline.

Disclosed herein is the use of any of the polypeptides, TNF-binding fragments or fusion proteins, as herein described, in the preparation of a medicament for the treatment or prevention of a condition mediated by TNF in an equine.

Taught herein is the use of any of the polypeptides, TNF-binding fragments or fusion proteins, as herein described, in the preparation of a medicament for the treatment or prevention of a condition mediated by TNF in a feline.

Contemplated herein is a method for treating or preventing a condition mediated by TNF in an equine, the method comprising the step of administering a therapeutically effective amount of any of the polypeptides, TNF-binding fragments or fusion proteins, as herein described, to an equine in need thereof.

Also contemplated herein is a method for treating or preventing a condition mediated by TNF in a feline, the method comprising the step of administering a therapeutically effective amount of any of the polypeptides, TNF-binding fragments or fusion proteins, as herein described, to a feline in need thereof.

Also provided herein is an agent capable of binding specifically to any of the TNFR polypeptides, TNF-binding fragments therefore or fusion proteins, as herein described.

Also enabled herein is a method for treating or preventing a condition associated with aberrant TNFR expression in a subject, the method comprising the step of administering a therapeutically effective amount of an agent capable of binding specifically to a TNFR p80 polypeptide, as herein described, to a subject in need thereof.

Also enabled herein is a method of diagnosing or monitoring in a subject a condition associated with aberrant expression of TNFR p80, the method comprising executing the step of analyzing a biological sample from a subject for the presence and/or level of TNFR p80, wherein the execution step comprises contacting the biological sample with an agent that is capable of binding specifically to a TNFR p80 polypeptide, as herein described.

Also disclosed herein is a method of diagnosing or monitoring in a subject a condition mediated at least in part by TNF, the method comprising executing the step of analyzing a biological sample from a subject for the presence and/or level of TNF, wherein the execution step comprises contacting the biological sample with a TNFR polypeptide, TNF-binding fragment therefore or fusion protein, as herein described.

### BRIEF DESCRIPTION OF THE FIGURES

FIG. 1 shows the DNA alignment of p80 TNFR sequences used for the design of sense PCR primer, EqDeg. The sequence of primer EqDeg is also shown. Also shown is the amino acid and nucleotide sequence of exon 2 of equine p80, with the EqGSP-1 and EqStdR reverse primer sequences.

FIG. 2A shows the alignment of the novel equine TNFR p80 exon 1 sequence, clone EqDegR2, with the corresponding human, mouse, dog and pig sequences.

FIG. 2B shows the Signal-P prediction of the signal peptide cleavage site for clone EqDegR2, where cleavage results in a mature equine TNFR p80 polypeptide comprising an N-terminal valine residue.

FIG. 3 shows the alignment of the predicted equine and feline TNFR p80 sequences with known sequences for pig, human, mouse and dog. Note location of exon 1 boundary (arrow).

FIG. 4 shows the Signal-P prediction analysis performed on the amino acid sequence of XM_005607617 (4A) and the human homolog, NM_001066 (4B).

FIG. 5 shows the correct predicted amino acid sequence of equine TNFR1A (p60) isoform.

FIG. 6 shows the nucleotide sequence encoding the equine TNFR p80 isoform.

FIG. 7 shows the N-terminal amino acid sequence of equine TNFR p80, aligned with the N-terminal amino acid sequence of dog, mouse, human and pig TNFR p80 isoforms. The site of TNFR signal sequence cleavage is depicted by the arrow.

FIG. 8 shows the nucleotide sequences of the degenerate PCR forward (sense) and reverse (antisense) primers FeGSP-1, FeGSP2 and FeGSP2nest. The template was feline first-strand cDNA primed with FeGSP2. The strike-through represents the incorrect 5′ sequence of the previously predicted amino acid sequence of feline TNFR p80, as represented, for example, by Genbank Accession No. XP_003989632.1 (SEQ ID NO:64).

FIG. 9 shows a micrograph of the PCR products produced by using the sense (EqDeg) and antisense (feline GSP-1 [GSP-1] or feline GSP2nest [GSP2nest]) primers, as previously shown in FIG. 8. The PCR product obtained with feline GSP-1 is approximately 276 bp (172 bp+104 bp [backbone]). The PCR product obtained with GSP2nest is approximately 356 bp (252 bp+backbone). The 100 bp ladder is shown on the far left of the micrograph.

FIG. 10 shows the alignment of the predicted N-terminal equine and feline TNFR p80 amino acid sequences with known amino acid sequences for pig, human, mouse and dog. The signal peptide cleavage site is shown by the arrow. The “cat_1” and “cat_2” amino acid sequences represent different clones that were obtained as a result of using a degenerate primer (see FIG. 1). The two clones differ by a single amino acid residue at position 6 within the signal sequence.

FIG. 11 shows the Signal-P prediction analysis performed on the predicted amino acid sequence for feline TNFR p80.

FIG. 12 shows the conditions used for the purification of the feline TNFR p80-Fc fusion peptide, NV-12 (a feline TNFRp80:HC1 fusion peptide comprising the extracellular domain of the mature feline TNFR p80 polypeptide and the hinge region, CH2 and CH3 domains of the feline IgG1 heavy chain constant region NV-12; SEQ ID NO:40) by MabSelect SuRe chromatography (FIG. 12A), HiTrap Q XL: anion exchange chromatography (FIG. 12B) and CHT Ceramic Hydroxyapatite purification (FIG. 12C).

FIG. 13 is a photomicrograph of an SDS-PAGE gel showing the feline TNFR p80-Fc fusion peptide, NV-12, purified by a 3-step process using (i) MabSelect SuRe chromatography, (ii) HiTrap Q XL: anion exchange chromatography and (iii) CHT Ceramic Hydroxyapatite purification. The first and third lanes of the SDS-PAGE gel photomicrograph show the molecular weight markers and the second and fourth lanes show protein bands of the purified NV-12 fusion peptide under non-reducing (NR; second lane) and reducing (R; fourth lane) conditions.

FIG. 14 shows the elution profile of the feline TNFR p80-Fc fusion peptide, NV-12, following the 3-step purification process, as shown in FIG. 12.

FIG. 15 shows the conditions used for the purification of the equine TNFR p80-Fc fusion peptide, NV-11 (an equine TNFRp80:HC2 fusion peptide comprising the ECD of mature equine TNFR p80 and an equine IgG2 heavy chain constant region; SEQ ID NO:14) by MabSelect SuRe chromatography (FIG. 15A) and Q Sepharose XL anion exchange chromatography (FIG. 15B).

FIG. 16 shows the photomicrographs of SDS PAGE gels showing the equine TNFR p80-Fc fusion peptide, NV-11, purified by a 2-step process using (i) MabSelect SuRe chromatography and (ii) Q Sepharose XL anion exchange chromatography under non-reducing (A) and reducing (B) conditions. The first lanes of each SDS-PAGE gel photomicrograph show the molecular weight markers. The second, third and fourth lanes of each SDS-PAGE gel photomicrograph show protein bands of NV-11 from the starting material (cell culture supernatant; second lanes), following purification by MabSelect SuRe chromatography (third lanes) and from the subsequent purification by Q Sepharose XL anion exchange chromatography (fourth lanes).

FIG. 17 shows the elution profile of the equine TNFR p80-Fc fusion peptide, NV-11, following the 2-step purification process, as shown in FIG. 15.

FIG. 18 shows the pharmacokinetics of NV-12. Approximately 2 mg/kg body weight of NV-12 was administered intravenously to two cats (subject nos. 1336 and 85364). Plasma NV-12 level was measured at various time points post-administration by enzyme-linked immunosorbent assay (ELISA). The terminal half-life of NV-12 was estimated to be about 3.6 days.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “a polypeptide” means one polypeptide or more than one polypeptide, unless otherwise indicated.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present.

The present disclosure provides polypeptides, fusion proteins, polynucleotides, compositions, kits, uses and methods for treating or preventing of a condition mediated, at least in part, by TNF in an equine or feline subject in need thereof, including an inflammatory condition. The polypeptides, fusion proteins, polynucleotides, compositions, kits, uses and methods herein described serve to sequester, abrogate, antagonise, inhibit or otherwise block TNF activity in an equine or feline subject, either systemically or at a particular anatomical location.

### Equine TNFR Polypeptides

The present invention is predicated, at least in part, on the inventors' surprising finding that the N-terminus of the equine tumour necrosis factor receptor (TNFR) p80 isoform comprises a signal peptide sequence that, when cleaved, gives rise to a mature form of equine TNFR p80 comprising an N-terminal valine (V) residue, more specifically, to an N-terminal VPAQV motif. This is to be contrasted with the earlier predicted equine p80 TNFR sequence (TNFRSF1B; Genbank Accession No. XM_005607617; SEQ ID NO:21), which the present inventors have determined has an incorrect leader sequence (MGEEAGVEGARASPFYSYLLHVEKSPITFPLQ; SEQ ID NO:20) derived from an incorrectly assigned exon 1 in the genome sequence. The incorrect leader sequence shown for XM_005607617 does not conform to the consensus and is not recognized as a signal peptide. This molecule, lacking a signal peptide, cannot be secreted from a cell in which it is expressed and is therefore non-functional.

By using a degenerate primer designed from the known DNA sequences that encode the human, mouse, pig and canine N-terminal sequences of the respective TNFR p80 isoforms, the present inventors have identified the correct N-terminal sequence for the p80 isoform of equine p80 TNFR. A signal peptide is predicted for the translated amino acid sequence, with cleavage of the signal sequence giving rise to a mature equine p80 TNFR polypeptide comprising an N-terminal VPAQV motif.

Thus, disclosed herein is an isolated equine tumor necrosis factor receptor (TNFR) polypeptide capable of binding to TNF, or a TNF-binding fragment of the polypeptide, wherein the polypeptide comprises an amino acid sequence of SEQ ID NO:1, or an amino acid sequence that has at least 60% similarity thereto after optimal alignment, and wherein the polypeptide or the TNF-binding fragment thereof comprises an N-terminal VPAQV motif, with the proviso that the polypeptide is not a mouse TNFR p80.

Also disclosed herein is an isolated equine tumor necrosis factor receptor (TNFR) polypeptide capable of binding to TNF, or a TNF-binding fragment of the polypeptide, wherein the polypeptide comprises an amino acid sequence of SEQ ID NO:1, or an amino acid sequence that has at least 60% identity thereto after optimal alignment, and wherein the polypeptide or the TNF-binding fragment thereof comprises an N-terminal VPAQV motif, with the proviso that the polypeptide is not a mouse TNFR p80.

The mature form of mouse TNFR p80 comprises an N-terminal VPAQVVL motif (GenBank Accession No. NP_035740; SEQ ID NO:53), taking into account the predicted signal sequence cleavage site. Thus, in an embodiment disclosed herein, the polypeptide or TNF-binding fragment thereof does not comprise an N-terminal VPAQVVL motif. In another embodiment, the polypeptide does not comprise the amino acid sequence of SEQ ID NOs:52 or 53.

In an embodiment disclosed herein, the polypeptide or TNF-binding fragment thereof is an equine TNFR p80 polypeptide, or TNF-binding fragment thereof, comprising an N-terminal VPAQVVF motif.

Without being bound by theory or a particular mode of action, the isolated TNFR p80 polypeptide or TNF-binding fragment thereof in accordance with the present invention, by virtue of having an N-terminal motif that shares sequence identity with the N-terminal amino acid sequence of the native equine TNFR p80 isoform, is expected to be less immunogenic following administration of the isolated polypeptide or fragment to an equine subject. This is to be contrasted, for example, to the equine TNFR p80 polypeptide that is based on the predicted polypeptide sequence represented by Accession No. XM_005607617 (SEQ ID NO:19), which is more likely to give rise to xenoantibodies in an equine host to which it is administered because it would comprise an N-terminal amino acid sequence that is foreign to equine; that is, it does not comprise the correct N-terminal amino acid sequence of native equine TNFR p80.

In an embodiment, the polypeptide comprises an amino acid sequence that has at least 85% similarity to SEQ ID NO:1 after optimal alignment. In another embodiment, the polypeptide comprises an amino acid sequence that has at least 85% identity to SEQ ID NO:1 after optimal alignment.

In an embodiment, the polypeptide is an equine TNFR polypeptide comprising the amino acid sequence of SEQ ID NO:1.

In an embodiment, the polypeptide comprises an amino acid sequence of SEQ ID NO:2, or an amino acid sequence that has at least 85% similarity thereto after optimal alignment. In another embodiment, the polypeptide comprises an amino acid sequence that has at least 85% identity to SEQ ID NO:2 after optimal alignment

In an embodiment, the polypeptide comprises an amino acid sequence of SEQ ID NO:3, or an amino acid sequence that has at least 85% similarity thereto after optimal alignment. In another embodiment, the polypeptide comprises an amino acid sequence that has at least 85% identity to SEQ ID NO:3 after optimal alignment.

In an embodiment, the polypeptide consists of an amino acid sequence selected from the group consisting of SEQ ID NO:1, SEQ ID NO:2 and SEQ ID NO:3.

As defined herein, the terms “equine” and “horse” are used interchangeably to a species belong to the subspecies with the trinomial name Equus ferus caballus, these being hooved (ungulate) mammals. Equines are a subspecies of the family Equidae and include any species classified therein and extends to over 300 breeds of horse known.

### Feline TNFR Polypeptides

The present invention is also predicated, at least in part, on the inventors' surprising finding that the N-terminus of feline TNFR p80 isoform comprises a signal peptide sequence that, when cleaved, gives rise to a mature form of feline TNFR p80 comprising an N-terminal valine (V) residue, more specifically, to an N-terminal VPAQV motif. This is to be contrasted with the predicted feline p80 TNFR sequence, as represented, for example, by Genbank Accession No. XP_003989632.1 (SEQ ID NO:64), which has an incorrect leader sequence (MSNCGHVLALSGVPPGWVWGCS; SEQ ID NO:65) derived from an incorrectly assigned exon 1 in the genome sequence. The incorrect leader sequence shown for XP_003989632.1 does not conform to the consensus and is not recognized as a signal peptide. As a consequence, this molecule, lacking a signal peptide, cannot be secreted from a cell in which it is expressed and is therefore non-functional.

By using a degenerate primer designed from the known DNA sequences that encode the N-terminal sequences of human, mouse, pig and canine TNFR p80 isoforms, the present inventors have identified the correct N-terminal sequence for the p80 isoform of feline TNFR. A signal peptide is predicted for the translated amino acid sequence, the cleavage of which gives rise to a mature feline TNFR p80 polypeptide comprising an N-terminal VPAQV motif.

Thus, disclosed herein is an isolated feline tumor necrosis factor receptor (TNFR) polypeptide capable of binding to TNF, or a TNF-binding fragment of the polypeptide, wherein the polypeptide comprises an amino acid sequence of SEQ ID NO:1, or an amino acid sequence that has at least 60% similarity thereto after optimal alignment, and wherein the polypeptide or the TNF-binding fragment thereof comprises an N-terminal VPAQV motif, with the proviso that the polypeptide is not a mouse TNFR p80.

Also disclosed herein is an isolated feline tumor necrosis factor receptor (TNFR) polypeptide capable of binding to TNF, or a TNF-binding fragment of the polypeptide, wherein the polypeptide comprises an amino acid sequence of SEQ ID NO:1, or an amino acid sequence that has at least 60% identity thereto after optimal alignment, and wherein the polypeptide or the TNF-binding fragment thereof comprises an N-terminal VPAQV motif, with the proviso that the polypeptide is not a mouse TNFR p80.

As noted elsewhere herein, the mature form of mouse TNFR p80 comprises an N-terminal VPAQVVL motif (GenBank Accession No. NP_035740; SEQ ID NO:53), taking into account the predicted signal sequence cleavage site. Thus, in an embodiment disclosed herein, the polypeptide or TNF-binding fragment thereof does not comprise an N-terminal VPAQVVL motif. In another embodiment, the polypeptide does not comprise the amino acid sequence of SEQ ID NOs:52 or 53.

In an embodiment disclosed herein, the polypeptide or TNF-binding fragment thereof is a feline TNFR p80 polypeptide, or a TNF-binding fragment thereof, comprising an N-terminal VPAQVAL motif.

Without being bound by theory or a particular mode of action, the isolated TNFR p80 polypeptide, or TNF-binding fragment thereof, in accordance with the present disclosure, by virtue of having an N-terminal VPAQV motif that shares sequence identity with the N-terminal amino acid sequence of the native feline TNFR p80 isoform, is expected to be less immunogenic following administration of the isolated polypeptide or fragment to a feline subject. This is to be contrasted, for example, to the predicted feline TNFR p80 polypeptide represented by Accession No. XP_003989632.1 (SEQ ID NO:64), which is more likely to give rise to xenoantibodies in a feline subject to which it is administered because it would comprise an N-terminal amino acid sequence that is foreign to feline; that is, it does not represent the correct N-terminal amino acid sequence of native feline TNFR p80.

In an embodiment disclosed herein, the polypeptide comprises an amino acid sequence of SEQ ID NO:36, or an amino acid sequence that has at least 85% similarity thereto after optimal alignment. In another embodiment disclosed herein, the polypeptide comprises an amino acid sequence that has at least 85% identity to SEQ ID NO:36 after optimal alignment.

In an embodiment disclosed herein, the polypeptide comprises an amino acid sequence of SEQ ID NO:37, or an amino acid sequence that has at least 85% similarity thereto after optimal alignment. In another embodiment disclosed herein, the polypeptide comprises an amino acid sequence that has at least 85% identity to SEQ ID NO:37 after optimal alignment

In an embodiment disclosed herein, the polypeptide consists of an amino acid sequence selected from the group consisting of SEQ ID NO:36 and SEQ ID NO:37.

The terms “immunogenic”, “immunogenicity” and the like, as used herein, typically refer to a measure of the ability of the polypeptide to elicit an immune response (humoral or cellular) when administered to a recipient. In an embodiment, the TNFR polypeptide or TNF-binding fragment thereof, according to the present invention, will have no immunogenicity; that is, that no xenoantibodies will be raised against it when administered to the target species (i.e., equine or feline), having regard to the N-terminal sequence of the polypeptide or TNF-binding fragment thereof. In another embodiment, the TNFR polypeptide or TNF-binding fragment thereof, according to the present invention, will have no detectable immunogenicity; that is, that no detectable xenoantibody titre will be raised against it when administered to the target species (i.e., equine or feline), having regard to the N-terminal sequence of the polypeptide or TNF-binding fragment thereof. In yet another embodiment, the TNFR polypeptide or TNF-binding fragment thereof, according to the present invention, will have low immunogenicity; that is, whilst there may be a detectable xenoantibody titre generated following its administration to the target species (i.e., equine or feline), the xenoantibody titre will be low such that it will not adversely affect the ability of the TNFR polypeptide or TNF-binding fragment thereof, as herein described, to sequester, inhibit, abrogate, antagonise or otherwise reduce the biological activity of TNF in the target species. The terms “xenoantibody” and “xenoantibodies” typically refer to an antibody which is raised by the host against an epitope which is foreign to the host.

It would be understood by persons skilled in the art that the closer the TNFR polypeptide sequence resembles the native TNFR polypeptide sequence of the target species (i.e., the higher the degree of sequence identity), the less likely it is to raise xenoantibodies in the target species to which it is administered. However, it would also be understood by persons skilled in the art that there may be instances where the generation of xenoantibodies (or risk of generating xenoantibodies) is inconsequential or unimportant to its intended use. The percentage identity of the TNFR polypeptide, or TNF-binding fragment thereof, according to the present invention, as compared to the native TNFR polypeptide sequence of the target species may therefore depend on the desired use or application. For instance, where the TNFR polypeptide, or TNF-binding fragment thereof, it is be administered to an equine or feline subject as a single dose (e.g., for the treatment or prophylaxis of an acute, TNF-mediated condition), the percentage identity, as compared to the native sequence of the TNFR p80 isoform of the target species (e.g., comprising the amino acid sequence represented by SEQ ID NO:3 for equine TNFR p80 and SEQ ID NOs:36 or 37 for feline TNFR p80) can be relatively low, since there is less risk that the administered the TNFR polypeptide or TNF-binding fragment would generate enough xenoantibodies in vivo that would neutralize, sequester, abrogate, antagonize, block or otherwise inhibit the ability of the TNFR polypeptide or fragment to bind to TNF in vivo.

Conversely, where the TNFR polypeptide or TFN-binding fragment thereof is to be administered to a target species as a series of multiple doses (e.g., for the long-term treatment or prophylaxis of a chronic, TNF-mediated condition), it may be beneficial (but not necessarily essential) that the TNFR polypeptide or TNF-binding fragment thereof comprises an amino acid sequence that has greater sequence identity to the native TNFR p80 amino acid sequence (as represented, for example, by SEQ ID NO:3 for equine TNFR p80 or SEQ ID NO:36 or 37 for feline TNFR p80).

The terms “polypeptide”, “peptide”, “protein” and the like are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide.

### Sequence Similarity and Identity

Reference to “at least 60%” includes 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence similarity or identity, for example, after optimal alignment or best fit analysis.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In an embodiment disclosed herein, nucleotide and amino acid sequence comparisons are made at the level of identity, rather than similarity.

Terms used to describe sequence relationships between two or more polynucleotides or polypeptides include “reference sequence”, “comparison window”, “sequence similarity”, “sequence identity”, “percentage sequence similarity”, “percentage sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of, for example, about 12 contiguous nucleotides that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (1997) Nucl. Acids. Res. 25:3389. A detailed discussion of sequence analysis can be found in Unit 19.3 of Ausubel et al. (1994-1998) In: Current Protocols in Molecular Biology, John Wiley & Sons Inc.

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

The term “sequence identity”, as used herein, includes exact identity between compared sequences at the nucleotide or amino acid level. This term is also used herein to include non-exact identity (i.e., similarity) at the nucleotide or amino acid level where any difference(s) between sequences are in relation to amino acids (or in the context of nucleotides, amino acids encoded by said nucleotides) that are nevertheless related to each other at the structural, functional, biochemical and/or conformational levels. For example, where there is non-identity (similarity) at the amino acid level, “similarity” includes amino acids that are nevertheless related to each other at the structural, functional, biochemical and/or conformational levels. In an embodiment, nucleotide and sequence comparisons are made at the level of identity rather than similarity. For example, leucine may be substituted for an isoleucine or valine residue. This may be referred to as a conservative substitution. In an embodiment, the amino acid sequences may be modified by way of conservative substitution of any of the amino acid residues contained therein, such that the modification has no or negligible effect on the binding specificity or functional activity of the modified polypeptide when compared to the unmodified polypeptide.

Sequence identity with respect to a polypeptide of the invention, or a TNF-binding fragment thereof or a fusion protein comprising any of the foregoing, as herein described, relates to the percentage of amino acid residues in the candidate sequence which are identical with the residues of the corresponding polypeptide, fragment or fusion protein after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology.

It would be understood by persons skilled in the art that the binding affinity of the TNFR polypeptide for TNF may vary depending on the amino acid sequence of the polypeptide. For example, in some instances, a TNFR polypeptide comprising a truncated form of the extracellular domain of TNFR p80 may have a lower binding affinity for equine or feline TNF as compared to, for example, the native TNFR p80 sequence or a TNFR p80 polypeptide comprising the entire extracellular domain of the respective equine or feline TNFR p80 (e.g., SEQ ID NO:3, 36 or 37), yet still be able to bind to equine or feline TNF and thereby sequester, inhibit, abrogate, antagonise or otherwise block its biological activity in vitro or in vivo. Methods for determining the binding affinity of the TNFR polypeptide for equine or feline TNF would be known to persons skilled in the art, illustrative examples of which are disclosed elsewhere herein. It would generally be desirable for the TNFR polypeptide to have a binding affinity for equine or feline TNF that is high enough so as to enable an effective amount of TNFR polypeptide of the present invention to be administered as a therapeutic or prophylactic agent to an equine or feline subject in need thereof in an effective amount that will sequester, inhibit, abrogate, antagonise or otherwise block the biological activity of equine or feline TNF in vivo. In an embodiment disclosed herein, the TNFR polypeptide binds to equine TNF-alpha with a binding affinity having an equilibrium-dissociation constant (K_{D}) of 1×10⁻⁸ or less.

### TNF-Binding Fragments

As used herein, the terms “TNF-binding fragment”, “binding fragment” and the like, when referring to a TNFR polypeptide, mean a portion of the TNFR polypeptide that retains the ability to bind to TNF (e.g., TNF-alpha and/or TNF-beta) in vivo or in vitro, in particular, to TNF of the same species (i.e., to equine and/or feline TNF). Suitable methods for determining whether a fragment of the TNFR polypeptide of the present invention is capable of binding to equine or feline TNF would be known to persons skilled in the art. For example, the fragment can be exposed to equine or feline TNF ligand for a period of time and under conditions that allow formation of an equine or feline TNF:TNFR-fragment complex and the resulting mixture separated by, e.g., gel electrophoresis, whereby a complex formed will be discernible from fragments and ligands based on size. In some embodiments, the fragment or ligand can be labelled with a detectable moiety (e.g., radioisotope or a fluorochrome) so as to assist in detection of an equine or feline TNF:TNFR complex. The ability of a TNF-binding fragment to bind equine and/or feline TNF can also be determined by using a suitable functional assay. For example, a method can be used to determine whether the TNF-binding fragment can bind to and inhibit the biological activity of equine or feline TNF. As used herein, the term “biological activity” refers to any one or more inherent biological properties of TNF (whether present naturally as found in vivo, or provided or enabled by recombinant means). Biological activity associated with equine or feline TNF would be known to persons skilled in the art. Illustrative examples include, but are not limited to, receptor binding and/or activation, induction of cell signalling or cell proliferation, inhibiting cell growth, induction of cytokine production, induction of apoptosis and enzymatic activity. Methods for determining the biological activity of equine TNF would also be known to persons skilled in the art. An illustrative example includes a cell-based assay. For example, cells that express TNFR and, upon exposure to equine or feline TNF, give rise to a measurable biological effect, are exposed to equine TNF in the presence or absence of the TNFR polypeptide of the present invention, or a TNF-binding fragment thereof. Measurements are then taken, or observations made, to determine whether the TNFR polypeptide or fragment thereof sequesters, inhibits, abrogates, antagonises or otherwise reduces the biological effect induced by equine or feline TNF on that cell

In an embodiment, the TNF-binding fragment comprises, consists of, or consists essentially of, a truncated TNFR polypeptide that lacks the transmembrane and cytoplasmic domains, yet retains an extracellular domain comprising a TNF-binding moiety that is capable of binding to equine or feline TNF.

In an embodiment disclosed herein, the TNF-binding fragment is a TNF-binding fragment of equine TNFR p80 comprising the CDR2 or CDR3 TNF-binding domains of equine TNFR p80. In an embodiment disclosed herein, the TNF-binding fragment is a TNF-binding fragment of equine TNFR p80 comprising a CDR2 domain and a CDR3 domain of equine TNFR p80. In an embodiment, the TNF-binding fragment consists of the amino acid sequence of SEQ ID NO:1, or an amino sequence having at least 60%, 65%, 70%, 75%, 80%, 85%, 90%, 93%, 95%, 96%, 97%, 98% or 99% sequence similarity thereto, wherein the TNF-binding fragment comprises an N-terminal VPAQV motif. In another embodiment, the TNF-binding fragment consists of the amino acid sequence of SEQ ID NO:1, or an amino sequence having at least 60%, 65%, 70%, 75%, 80%, 85%, 90%, 93%, 95%, 96%, 97%, 98% or 99% sequence identity thereto, wherein the TNF-binding fragment comprises an N-terminal VPAQV motif. In an embodiment, the TNF-binding fragment comprises an N-terminal VPAQVV motif. In another embodiment, the TNFR polypeptide comprises an N-terminal VPAQVVF motif.

In an embodiment, the TNF-binding fragment consists of the amino acid sequence of SEQ ID NO:3.

In yet another embodiment disclosed herein, the TNF-binding fragment is a TNF-binding fragment of feline TNFR p80 comprising the CDR2 or CDR3 TNF-binding domains of feline TNFR p80. In an embodiment disclosed herein, the TNF-binding fragment is a TNF-binding fragment of feline TNFR p80 comprising a CDR2 domain and a CDR3 domain of feline TNFR p80. In an embodiment, the TNF-binding fragment consists of the amino acid sequence of SEQ ID NO:36 or SEQ ID NO:37, or an amino sequence having at least 60%, 65%, 70%, 75%, 80%, 85%, 90%, 93%, 95%, 96%, 97%, 98% or 99% sequence similarity thereto, wherein the TNF-binding fragment comprises an N-terminal VPAQV motif. In another embodiment, the TNF-binding fragment consists of the amino acid sequence of SEQ ID NO:36 or SEQ ID NO:37, or an amino sequence having at least 60%, 65%, 70%, 75%, 80%, 85%, 90%, 93%, 95%, 96%, 97%, 98% or 99% sequence identity thereto, wherein the TNF-binding fragment comprises an N-terminal VPAQV motif. In another embodiment, the TNF-binding fragment comprises an N-terminal VPAQVA motif. In another embodiment, the TNFR polypeptide comprises an N-terminal VPAQVAL motif.

In an embodiment, the TNF-binding fragment consists of the amino acid sequence of SEQ ID NO:36 or SEQ ID NO:37.

Typically, the TNFR polypeptide or TNF-binding fragment thereof, as herein described, is a TNF-neutralising polypeptide or fragment. As used herein, the term “neutralising” describes a TNFR polypeptide or fragment comprising a TNF-binding moiety; that is, a TNFR polypeptide moiety that is capable of binding to equine or feline TNF and sequestering, inhibiting, abrogating, antagonising or otherwise blocking the biological activity of TNF in vivo or in vitro. In an embodiment, the neutralising TNFR polypeptide or fragment, which may also be referred to as a TNF antagonist, an antagonistic TNFR polypeptide, or a blocking TNFR polypeptide, specifically and preferably selectively, binds to equine TNF and inhibits one or more biological activities of equine TNF. For example, the neutralising TNFR polypeptide may inhibit the binding of equine TNF to its target receptor, such as the cell membrane bound TNF Receptor 1 (TNFR1) receptor (CD120a). In another embodiment, the neutralising TNFR polypeptide or fragment specifically and preferably selectively binds to feline TNF and inhibits one or more biological activities of feline TNF. For example, the neutralising TNFR polypeptide may inhibit the binding of feline TNF to its target receptor, such as the cell membrane bound TNF Receptor 1 (TNFR1) receptor (CD120a).

The phrase “specifically binds to” and the like refer to the binding of the TNFR polypeptide or fragment, as herein described, to TNF, specifically to equine or feline TNF, which may be present amongst a heterogeneous population of other material, including other polypeptides and proteins. Hence, when present in specific immunoassay conditions, the TNFR polypeptide of the present invention, or TNF-binding fragments thereof, will bind to equine or feline TNF, and will not bind (or not bind in a significant amount) to other proteins that may be present.

### Fusion Proteins

Also disclosed herein is a fusion protein comprising a TNFR polypeptide, or a TNF-binding fragment thereof, as herein described, linked to or otherwise attached to a fusion partner.

The fusion protein disclosed herein may be a chimeric fusion protein; that is, comprising at least two domains that are derived from different species. These domains are brought together in the chimeric, dimeric or fusion polypeptide. For instance, the TNFR polypeptide, or TNF-binding fragment thereof, as herein described, can be an equine TNFR polypeptide, or TNF-binding fragment thereof, and the fusion partner can be derived from a non-equine species (i.e., comprising an amino acid sequence derived from a species other than equine). Similarly, the TNFR polypeptide, or TNF-binding fragment thereof, as herein described, can be a feline TNFR polypeptide, or TNF-binding fragment thereof, and the fusion partner is derived from a non-feline species (i.e., comprising an amino acid sequence derived from a species other than feline). In an embodiment disclosed herein, the chimeric fusion protein comprises an equine TNFR polypeptide, or TNF-binding fragment thereof, linked to a fusion partner derived from feline. In another embodiment disclosed herein, the chimeric fusion protein comprises a feline TNFR polypeptide, or TNF-binding fragment thereof, linked to a fusion partner derived from equine. It will be understood, however, that where the fusion protein is to be used as a therapeutic or prophylactic agent (or as part of a therapeutic or prophylactic agent) that is to be administered to a target species, then the fusion protein would comprise a fusion partner that is native to the target species, thereby minimising or avoiding immunogenicity that would otherwise be associated with the use of a fusion protein comprising a heterologous component (i.e., a component derived from a species other than the target species). Thus, in an embodiment disclosed herein, the fusion protein comprises an equine TNFR polypeptide, or TNF-binding fragment thereof, as herein described, linked to a fusion partner derived from equine. In another embodiment disclosed herein, the fusion protein comprises a feline TNFR polypeptide, or TNF-binding fragment thereof, as herein described, linked to a fusion partner derived from feline.

In an embodiment disclosed herein, the TNFR polypeptide, or TNF-binding fragment thereof, is linked to, attached to or otherwise associated with, the fusion partner at the C-terminus or at the N-terminus of said polypeptide or fragment. It will be understood that the preferred linkage, attachment or association of the fusion partner will be at a position such that the fusion partner does not inhibit or otherwise block the ability of said polypeptide or fragment to bind to TNF. In an embodiment disclosed herein, the TNFR polypeptide, or TNF-binding fragment thereof, is linked to, attached to or otherwise associated with, the fusion partner at the C-terminus of said polypeptide or fragment,

The TNFR polypeptide, or TNF-binding fragment thereof, can be linked to, attached to or otherwise associated with, the fusion partner by any suitable means known to persons skilled in the art. In illustrative examples, the fusion partner can be linked to the TNFR polypeptide or TNF-binding fragment by a covalent bond or by non-covalent means. In another illustrative example, the fusion partner can be linked to the TNFR polypeptide or TNF-binding protein by recombinant means (i.e., the fusion protein is produced by recombinant means; e.g., recombinant DNA technology), as is described elsewhere herein.

The choice of fusion partner will be dictated, at least in part, by the proposed application or use of the fusion protein. For example, in some embodiments, the fusion partner will assist in targeting the TNFR polypeptide, or TNF-binding fragment thereof, to a desired anatomical site of action (e.g., a site of aberrant TNF activity). Suitable fusion partners for this purpose would be known to persons skilled in the art. In an illustrative example, the fusion partner will be a targeting moiety capable of binding to a target ligand (e.g., a protein, antigen, epitope, nucleic acid molecule) that is expressed by a cell or tissue. Examples of suitable targeting moieties include receptors (e.g., soluble receptors) and antibodies or ligand-binding fragments thereof (e.g. single chain Fv (scFv), Fab, etc) that are capable of binding specifically to the target ligand. In some embodiments, the fusion partner will assist to reduce the clearance of the TNFR polypeptide, or TNF-binding fragment thereof, to which it is linked, in vivo. In other embodiments, the fusion partner will assist to improve the stability of the TNFR polypeptide, or TNF-binding fragment thereof, to which it is linked, in vivo. Suitable fusion partners capable of improving the stability of the TNFR polypeptide, or TNF-binding fragment thereof, to which it is linked will be known to persons skilled in the art. An illustrative example includes an Fc region of an immunoglobulin molecule (e.g., IgG), or a portion thereof (e.g., an immunoglobulin heavy chain constant region). Without being bound by theory or any particular mode of action, it is understood that the Fc region of an immunoglobulin (e.g., IgG) heavy chain constant region, or a portion thereof, can improve the solubility, half-life and/or stability of the TNFR polypeptide, in vitro and in vivo. Employing an Fc region, or a potion thereof, also allows for ease of purification, for example, by Protein-G/Protein-A affinity chromatography.

A fusion partner derived from any species can be used in the fusion protein of the present invention, such as human, canine, feline, porcine and murine Fc immunoglobulin regions, or potions thereof. In some instances, as noted elsewhere herein, it may desirable to generate a fusion protein that minimises or avoids the risk of generating xenoantibodies (i.e., immunogenicity) when administered to a target species, such as equine or feline, for example, by using a fusion partner that is substantially immunologically inert so as to minimise or avoid immunogenicity when administered to a target species; that is, the risk of generating xenoantibodies when the fusion protein of the present invention is administered to a target species such as equine or feline will be minimised or avoided. This is particularly advantageous where the fusion protein is administered as multiple doses to a subject over a period of time (e.g., for the treatment of a chronic condition that is mediated, at least in part, by TNF).

In an embodiment disclosed herein, the fusion partner comprises a CH2 domain and/or a CH3 domain of an immunoglobulin heavy chain constant region.

As noted elsewhere herein, where the fusion protein is to be used as a therapeutic or prophylactic agent (or as part of a therapeutic or prophylactic agent) that is to be administered to a target species, then the fusion partner is native to the target species, thereby minimising or avoiding immunogenicity that would otherwise be associated with the use of a fusion protein comprising a heterologous component (i.e., a component derived from a species other than the target species). Thus, where the fusion protein is to be administered to an equine, one would use a CH2 domain and/or a CH3 domain of an immunoglobulin heavy chain constant region that is native to equine (e.g., derived from equine immunoglobulin). Similarly, where the fusion protein is to be administered to a feline, one would use a CH2 domain and/or a CH3 domain of an immunoglobulin heavy chain constant region that is native to feline (e.g., derived from feline immunoglobulin).

In an embodiment disclosed herein, the fusion partner comprises a CH2 domain and/or a CH3 domain of a heavy chain constant region of an equine IgG. The amino acid sequence of equine immunoglobulin heavy chain constant regions, including equine IgG and isotypes thereof, would be known to persons skilled in the art. With respect to equine immunoglobulin gamma (IgG), seven distinct heavy chain constant domain isotypes have been identified—IgG1 to IgG7. Thus, in an embodiment, the fusion partner comprises a CH2 domain and/or a CH3 domain of a heavy chain constant region of an equine IgG isotype selected from the group consisting of IgG1 (SEQ ID NO:7), IgG2 (SEQ ID NO:8), IgG3 (SEQ ID NO:9), IgG4 (SEQ ID NO:10), IgG5 (SEQ ID NO:11), IgG6 (SEQ ID NO:12) and IgG7 (SEQ ID NO:13). In an embodiment disclosed herein, the fusion partner comprises a CH2 domain and a CH3 domain of a heavy chain constant region of an equine Ig molecule (e.g., an equine IgG molecule), as herein described.

In another embodiment disclosed herein, the fusion partner comprises a CH2 domain and/or a CH3 domain of a heavy chain constant region of a feline immunoglobulin molecule. The amino acid sequence of feline immunoglobulin heavy chain constant regions, including their CH2 and CH3 domains, would be known to persons skilled in the art, as shown, for example, in SEQ ID NOs:43-45 disclosed herein. In an embodiment, the feline immunoglobulin is a feline IgG. In an embodiment disclosed herein, the fusion partner comprises a CH2 domain and a CH3 domain of a heavy chain constant region of a feline Ig molecule (e.g., a feline IgG molecule), as herein described.

In another embodiment disclosed herein, the fusion partner further comprise an immunoglobulin heavy chain hinge region. Suitable hinge regions derived from an immunoglobulin heavy chain constant region would be known to persons skilled in the art. It will be understood, however, that where the fusion protein is to be used as a therapeutic or prophylactic agent (or as part of a therapeutic or prophylactic agent) that is to be administered to a target species, then it would be desirable that the hinge region is native to the target species, thereby minimising or avoiding the immunogenicity that would otherwise be associated with the use of a heterologous hinge region (i.e., a hinge region derived from immunoglobulin of a species other than the target species). Thus, where the fusion protein is to be administered to an equine, it is desirable to use a hinge region that is native to equine (e.g., derived from equine immunoglobulin). Similarly, where the fusion protein is to be administered to a feline, it is desirable to use a hinge region that is native to feline (e.g., derived from feline immunoglobulin).

In an embodiment disclosed herein, the hinge region is an equine immunoglobulin hinge region. Suitable equine immunoglobulin hinge regions would be known to persons skilled in the art, an illustrative example of which includes equine IgG and subtypes thereof. Thus, in an embodiment disclosed herein, the hinge region is an equine IgG hinge region. In an embodiment, the equine IgG hinge region comprises an amino acid sequence of the hinge region shown in SEQ ID NOs:7-13 and 54-60 or an amino acid sequence that has at least 85% similarity to any of SEQ ID NOs:7-13 and 54-60 after optimal alignment. In another embodiment, the equine IgG hinge region comprises an amino acid sequence that has at least 85% identity to any of SEQ ID NOs:7-13 and 54-60 after optimal alignment. In an embodiment, the equine IgG hinge region consists of the amino acid sequence of the hinge region shown in any one of SEQ ID NOs:7-13 and 54-60. In another embodiment, the hinge region is an equine IgG hinge region comprising an amino acid sequence of SEQ ID NO:35, or an amino acid sequence that has at least 85% similarity thereto after optimal alignment. In another embodiment, the hinge region is an equine IgG hinge region comprising an amino acid sequence that has at least 85% identity to SEQ ID NO:35 after optimal alignment. In another embodiment, the hinge region consists of the amino acid sequence of SEQ ID NO:35.

In an embodiment disclosed herein, the fusion partner comprises an amino acid sequence selected from the group consisting of SEQ ID NOs:7-13 and 54-60, or an amino acid sequence that has at least 85% similarity to any of SEQ ID NOs:7-13 and 54-60 after optimal alignment. In another embodiment disclosed herein, the fusion partner comprises an amino acid sequence that has at least 85% identity to any of SEQ ID NOs:7-13 and 54-60 after optimal alignment. In another embodiment, the fusion partner consists of the amino acid sequence selected from the group consisting of SEQ ID NOs:7-13 and 54-60.

In an embodiment disclosed herein, the fusion protein comprises an amino acid selected from the group consisting of SEQ ID NOs: 14, 15 and 21-34, or an amino acid sequence that has at least 85% similarity to any of SEQ ID NOs: 14, 15 and 21-34 after optimal alignment. In another embodiment disclosed herein, the fusion protein comprises an amino acid sequence that has at least 85% identity to any of SEQ ID NOs: 14, 15 and 21-34 after optimal alignment. In an embodiment disclosed herein, the fusion protein consists of an amino acid selected from the group consisting of SEQ ID NOs: 14, 15 and 21-34.

In another embodiment disclosed herein, the hinge region is a feline immunoglobulin hinge region. Suitable feline immunoglobulin hinge regions would be known to persons skilled in the art, an illustrative example of which includes feline IgG and subtypes thereof. Thus, in an embodiment disclosed herein, the hinge region is a feline IgG hinge region. In an embodiment, the feline IgG hinge region comprises an amino acid sequence of the hinge region shown in SEQ ID NOs:43-45 and 61-63 or an amino acid sequence that has at least 85% similarity to any of SEQ ID NOs:43-45 and 61-63 after optimal alignment. In another embodiment, the feline IgG hinge region comprises an amino acid sequence that has at least 85% identity to any of SEQ ID NOs:43-45 and 61-63 after optimal alignment. In an embodiment, the feline IgG hinge region consists of the amino acid sequence of the hinge region shown in any one of SEQ ID NOs:43-45 and 61-63.

In an embodiment disclosed herein, the fusion partner comprises an amino acid sequence selected from the group consisting of SEQ ID NOs:43-45 and 61-63, or an amino acid sequence that has at least 85% similarity to any of SEQ ID NOs:43-45 and 61-63 after optimal alignment. In another embodiment disclosed herein, the fusion partner comprises an amino acid sequence that has at least 85% identity to any of SEQ ID NOs:43-45 and 61-63 after optimal alignment. In another embodiment disclosed herein, the fusion partner consists of an amino acid sequence selected from the group consisting of SEQ ID NOs:43-45 and 61-63.

In an embodiment disclosed herein, the fusion protein comprises an amino acid sequence selected from the group consisting of SEQ ID NOs: 40 and 46-51, or an amino acid sequence that has at least 85% similarity to any of SEQ ID NOs: 40 and 46-51 after optimal alignment. In another embodiment disclosed herein, the fusion protein comprises an amino acid sequence that has at least 85% identity to any of SEQ ID NOs: 40 and 46-51 after optimal alignment. In an embodiment disclosed herein, the fusion protein consists of an amino acid selected from the group consisting of SEQ ID NOs: 40 and 46-51.

The TNFR polypeptide, or TNF-binding fragment thereof, may be linked to the fusion partner by way of a covalent linkage. In some embodiments, a linker, such as a polypeptide linker, may be used to covalently link the TNFR polypeptide, or TNF-binding fragment thereof, to the fusion partner, to form the fusion protein, which is also referred to herein as a fusion polypeptide, chimeric fusion polypeptide or an immunoconjugate. In some embodiments, a linker region is not required, for example, where structural flexibility is conferred by a hinge region of an immunoglobulin heavy chain constant region, where such hinge region (or fragment thereof) is present in the fusion protein of the present invention, as herein described. In another embodiment, the fusion protein further comprises a linker functionally interposed between the TNFR polypeptide and the fusion partner.

By “linker” is meant a molecule or group of molecules (such as a monomer or polymer) that connects two molecules (i.e., the TNFR polypeptide, or TNF-binding fragment thereof, and the fusion partner) and serves to place the two molecules in a desirable configuration.

In an embodiment, the fusion protein of the present invention comprises a fusion partner that is attached to the C-terminus of the TNFR polypeptide, or TNF-binding fragment thereof. In another embodiment, the fusion protein comprises a fusion partner that is attached to the N-terminus of the TNFR polypeptide, or TNF-binding fragment thereof, without adversely affecting the ability of the TNFR polypeptide to bind to equine TNF and sequester, abrogate, antagonise, inhibit or otherwise block its biological activity in vitro or in vivo.

The present invention contemplates any suitable method known to persons skilled in the art for attaching (e.g., conjugating, linking or fusing) the TNFR polypeptide, or TNF-binding fragment thereof, to a fusion partner, non-limiting examples of which include:

(i) Use of a carbodiimide or other suitable coupling agent to form an amide linkage between a free amino group of the TNFR polypeptide, or TNF-binding fragment thereof, and a free carboxyl group of the fusion partner or a linker peptide fused or otherwise attached to the fusion partner;
(ii) Use of a carbodiimide or other suitable coupling agent to form an amide linkage between a free amino group of the fusion partner and a free carboxyl group of the TNFR polypeptide, or TNF-binding fragment thereof, or a linker peptide fused or otherwise attached to the TNFR polypeptide, or TNF-binding fragment thereof;
(iii) Use of MBS (m-maleimidonemzoic acid N-hydroxysuccinimide ester) to link via the thiol group of a cysteine added to the C-terminus of the TNFR polypeptide, or TNF-binding fragment thereof or to the C-terminus of a linker peptide attached or otherwise fused to the TNFR polypeptide, or TNF-binding fragment thereof, to a free amino group at the N-terminus of the fusion partner;
(iv) Use of MBS (m-maleimidonemzoic acid N-hydroxysuccinimide ester) to link via the thiol group of a cysteine added to the C-terminus of the fusion partner or to the C-terminus of a linker peptide attached or otherwise fused to the fusion partner, to a free amino group at the N-terminus of the TNFR polypeptide, or TNF-binding fragment thereof;
(v) Use of glutaraldehyde to conjugate a free amino group on the fusion partner or on a linker protein fused or otherwise attached to the fusion partner and a free amino group on the TNFR polypeptide, or TNF-binding fragment thereof or on a linker protein fused or otherwise attached to the TNFR polypeptide, or TNF-binding fragment thereof;
(vi) Use of glutaraldehyde to conjugate a free amino group on the TNFR polypeptide, or TNF-binding fragment thereof, or on a linker protein fused or otherwise attached to the TNFR polypeptide, or TNF-binding fragment thereof, and a free amino group on the fusion partner or on a linker protein fused or otherwise attached to the fusion partner;
(vii) Use of SO₂Cl or triazoles to couple an amine of the fusion partner to an aldehyde-containing moiety of the TNFR polypeptide, or TNF-binding fragment thereof; or
(viii) Use of SO₂Cl or triazoles to couple an amine of the TNFR polypeptide, or TNF-binding fragment thereof to an aldehyde-containing moiety of the fusion partner.

Other suitable methods of attaching a fusion partner to the TNFR polypeptide, or TNF-binding fragment thereof, would be known to persons skilled in the art. In an illustrative example, the TNFR polypeptide, or a TNF-binding fragment thereof, is conjugated to the fusion partner, either with or without linker peptides extending from the C- or N-termini, by recombinant fusion or by peptide-peptide covalent conjugation. Linkers could also be prepared with several spaced reactive residues via which to conjugate multiple sulfonylurea compounds.

In an embodiment, the fusion protein, with or without an intervening linker, can be made by peptide synthesis or by recombinant expression using any suitable prokaryotic or eukaryotic expression system (including but not limited to bacterial, yeast, insect and mammalian cells), as known in the art.

In an embodiment disclosed herein, the fusion protein comprises an N-terminal VPAQV motif, with the proviso that the N-terminal motif is not VPAQVVL. In another embodiment, the fusion protein comprises an N-terminal VPAQVVF motif, which corresponds to the N-terminal motif of the mature equine TNFR p80 isoform (i.e., subsequent to cleavage of the signal sequence). In yet another embodiment, the fusion protein comprises an N-terminal VPAQVAF, which corresponds to the N-terminal motif of the mature feline TNFR p80 isoform (i.e., subsequent to cleavage of the signal sequence).

In an embodiment, the fusion proteins disclosed herein will have no immunogenicity in the target species; that is, no xenoantibodies will be raised against it when administered to the target species. In another embodiment, the fusion proteins disclosed herein will have no detectable immunogenicity; that is, no detectable xenoantibody titre will be raised against it when administered to the target species. In yet another embodiment, the fusion proteins disclosed herein will have low immunogenicity; that is, whilst there may be a detectable xenoantibody titre generated following its administration to the target species, the xenoantibody titre will be low enough such that it will not adversely affect the ability of the fusion protein to sequester, inhibit, abrogate, antagonise or otherwise reduce the biological activity of TNF in the recipient. The terms “xenoantibody” and “xenoantibodies” typically refer to an antibody which is raised by the recipient against an epitope which is foreign to the recipient

In an embodiment, the fusion protein is capable of binding to equine TNF and sequestering, inhibiting, abrogating, antagonising or otherwise blocking its biological activity in vitro or in vivo. In another embodiment, the fusion protein is capable of binding to and sequestering, inhibiting, abrogating, antagonising or otherwise blocking the biological activity of equine TNF-alpha (TNF-α) and/or TNF-beta (TNF-β). In an embodiment, the fusion protein is capable of binding to and sequestering, inhibiting, abrogating, antagonising or otherwise blocking the biological activity of equine TNF-alpha.

In an embodiment, the fusion protein is capable of binding to feline TNF and sequestering, inhibiting, abrogating, antagonising or otherwise blocking its biological activity in vitro or in vivo. In another embodiment, the fusion protein is capable of binding to and sequestering, inhibiting, abrogating, antagonising or otherwise blocking the biological activity of feline TNF-alpha (TNF-α) and/or TNF-beta (TNF-β). In an embodiment, the fusion protein is capable of binding to and sequestering, inhibiting, abrogating, antagonising or otherwise blocking the biological activity of feline TNF-alpha.

It would be understood by persons skilled in the art that the binding affinity of the TNFR polypeptide moiety of the fusion protein may vary depending on the sequence of that moiety. For example, in some instances, a TNFR polypeptide comprising a truncated form of the extracellular domain of the TNFR p80 isoform may have a lower binding affinity for TNF as compared to, for example, the native TNFR p80 polypeptide or a TNFR p80 polypeptide comprising the entire extracellular domain of TNFR p80, yet still be able to bind to TNF and thereby sequester, inhibit, abrogate, antagonise or otherwise block its biological activity in vitro or in vivo. Methods for determining the binding affinity of the fusion protein of the present invention for TNF (e.g., for equine or feline TNF) would be known to persons skilled in the art, as noted elsewhere herein. It would generally be desirable for the TNFR polypeptide of the fusion protein to have a binding affinity for the target TNF that is high enough so as to enable an effective amount of the fusion protein to be administered to the target so that it will sequester, inhibit, abrogate, antagonise or otherwise block the biological activity of TNF in the target (in vivo). In an embodiment disclosed herein, the fusion protein binds to equine TNF-alpha with a binding affinity having an equilibrium-dissociation constant (K_{D}) of 1×10⁻⁸ or less. In another embodiment disclosed herein, the fusion protein binds to feline TNF-alpha with a binding affinity having an equilibrium-dissociation constant (K_{D}) of 1×10⁻⁸ or less.

The fusion proteins, as herein disclosed, will generally exhibit improved stability and/or in vivo half life. Furthermore, the fusion proteins overcome, at least in part, problems associated with administering hitherto known anti-TNF compounds to a target species, most specifically by limiting the production of xenoantibodies thereagainst, when administered to the target.

In some embodiments, where the fusion partner comprises a CH2 domain and/or CH3 domain of an immunoglobulin heavy chain constant region, the fusion protein does not mediate any downstream effector function, such as complement recruitment, fixation and activation, ADCC and Fc receptor binding and activation. Mutations, substitutions and additions may be made to the amino acid sequence of the fusion partner to ensure that downstream effector functions are minimised or do not occur. In other embodiments, where the fusion partner comprises a CH2 domain and/or CH3 domain of an immunoglobulin heavy chain constant region, the immunoglobulin isotype may be selected on the base of their desirable properties in not mediating downstream effector functions. In other embodiments, modifications to the amino acid sequence or to the amino acid residues of a native immunoglobulin heavy chain constant region, or to the CH2 and/or CH3 domains derived therefrom, can be made. Such modifications may involve the addition, substitution or deletion of one or more amino acid residues. In some embodiments, the amino acid changes are performed in order to modify the functional characteristics of the fusion partner; for example, so as to inhibit or otherwise remove any effector function that may be associated with that sequence. In an illustrative example, amino acid modifications can be performed to prevent downstream effector functions mediated by the immunoglobulin heavy chain constant region, or by the CH2 and/or CH3 domains derived therefrom (e.g., by preventing the ability of the fusion partner to bind to Fc receptors, activate complement and/or induce ADCC). Modifications may also be made to the amino acid residues of the fusion partner in order to modify the half-life of the fusion protein in vivo.

In other embodiments, where the fusion partner comprises a CH2 domain and/or CH3 domain of an immunoglobulin heavy chain constant region, the fusion protein comprises downstream effector function, such as complement recruitment, fixation and activation, ADCC and Fc receptor binding and activation. Mutations, substitutions and additions may be made to the amino acid sequence of the fusion partner to enhance downstream effector functions. In other embodiments, where the fusion partner comprises a CH2 domain and/or CH3 domain of an immunoglobulin heavy chain constant region, the immunoglobulin isotype may be selected on the base of their desirable properties in mediating downstream effector functions. In other embodiments, modifications to the amino acid sequence or to the amino acid residues of a native immunoglobulin heavy chain constant region, or to the CH2 and/or CH3 domains derived therefrom, can be made. Such modifications may involve the addition, substitution or deletion of one or more amino acid residues. In some embodiments, the amino acid changes are performed in order to modify the functional characteristics of the fusion partner; for example, so as to enhance downstream effector function associated with that sequence.

In other embodiments, the fusion partner comprises a CH2 domain and/or CH3 domain of an immunoglobulin heavy chain constant region that has an altered glycosylation pattern. For example, the fusion partner can be altered to increase or decrease the extent to which the amino acid residues of the fusion partner are glycosylated. Glycosylation of polypeptides is typically either N-linked or O-linked. N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose or xylose to a hydroxyamino acid, most commonly serine or threonine, although 5-hydroxyproline or 5-hydroxylysine may also be used.

In some embodiments, the fusion protein can be PEGylated by reacting the fusion protein with a polyethylene glycol (PEG) derivative. In some embodiments, the fusion protein is defucosylated and therefore lacks fucose residues.

In some embodiments, modifications may be accomplished by selecting substitutions that affect (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (A. L. Lehninger, in Biochemistry, 2^{nd} Ed., 73-75, Worth Publishers, New York (1975)): (1) non-polar: Ala (A), VaI (V), Leu (L), Ile (I), Pro (P), Phe (F), Trp (W), Met (M); (2) uncharged polar: GIy (G), Ser (S), Thr (T), Cys (C), Tyr (Y), Asn (N), Gln (Q); (3) acidic: Asp (D), GIu (E); and (4) basic: Lys (K), Arg (R), His (H). Alternatively, naturally occurring residues may be divided into groups based on common side-chain properties: (1) hydrophobic: Norleucine, Met, Ala, VaI, Leu, Ile; (2) neutral hydrophilic: Cys, Ser, Thr, Asn, Gln; (3) acidic: Asp, GIu; (4) basic: His, Lys, Arg; (5) residues that influence chain orientation: GIy, Pro; and (6) aromatic: Trp, Tyr, Phe. Non-conservative substitutions will entail exchanging a member of one of these classes for another class. Such substituted residues may also be introduced into the conservative substitution sites or into the remaining (e.g. non-conserved) sites.

Typically, the fusion protein of the present invention is a TNF-neutralising fusion protein. In this context, the term “neutralising” describes a fusion protein comprising a TNF-binding moiety (i.e., the TNFR polypeptide moiety) that is capable of binding to TNF of a target species (e.g., equine and/or feline TNF) and sequestering inhibiting abrogating antagonising or otherwise blocking the biological activity of the target TNF. In an embodiment, the neutralising fusion protein, which may also be referred to as a TNF antagonist, an antagonistic fusion protein, or a blocking fusion protein, specifically and preferably selectively binds to TNF of the target species and inhibits one or more biological activities of TNF in the target species. For example, the neutralising fusion protein may inhibit the binding of equine TNF to its target receptor, such as the cell membrane bound TNF Receptor 1 (TNFR1) receptor (CD120a). Similarly, the neutralising fusion protein may inhibit the binding of feline TNF to its target receptor, such as the cell membrane bound TNF Receptor 1 (TNFR1) receptor (CD120a).

The phrase “specifically binds to” and the like refers to the binding of the fusion protein, to TNF in a target species, such as equine and/or feline TNF, which may be present amongst a heterogeneous population of other material, including other polypeptides and proteins. Hence, when present in specific immunoassay conditions, the fusion protein of the present invention will bind to the target TNF, and not bind in a significant amount to other proteins that may be present in a sample.

As used herein, the term “biological activity” refers to any one or more inherent biological properties of a molecule (whether present naturally as found in vivo, or provided or enabled by recombinant means). Biological properties associated with TNF would be known to persons skilled in the art. Illustrative examples include, but are not limited to, receptor binding and/or activation, induction of cell signalling or cell proliferation, inhibiting cell growth, induction of cytokine production, induction of apoptosis and enzymatic activity. Methods for determining the biological activity of TNF would also be known to persons skilled in the art. An illustrative example includes a cell-based assay. For example, cells that express TNFR and, upon exposure to TNF, give rise to a measurable biological effect, are exposed to TNF in the presence or absence of the TNFR polypeptide and/or the fusion protein of the present invention, including TNF-binding fragments thereof, and/or a polynucleotide encoding the same. Measurements are then taken, or observations made, to determine whether the TNFR polypeptide, fusion protein, fragment and/or polynucleotide inhibits the biological effect mediated by TNF on that cell.

The terms “consists essentially of” or “consisting essentially of”, as used herein, typically mean that the TNFR polypeptide, TNF-binding fragments thereof, or the fusion protein, may have additional features or elements beyond those described provided that such additional features or elements do not materially affect their ability to bind specificity to TNF. That is, the TNFR polypeptide, TNF-binding fragment thereof or fusion protein may have additional features or elements that do not significantly interfere with the ability of the TNF-binding moiety to bind to TNF of a target species (e.g., equine or feline TNF) and sequester, inhibit, antagonise, abrogate or otherwise reduce the biological activity of the TNF molecule. Such modifications may be introduced into the amino acid sequence in order to reduce the immunogenicity of the TNFR polypeptide, fragment or fusion protein. For example, a polypeptide consisting essentially of a specified sequence may contain one, two, three, four, five or more additional, deleted or substituted amino acids, at either end or at both ends of the sequence provided that these amino acids do not interfere with binding of TNF to the TNF-binding moiety and sequester, inhibit, antagonise, abrogate or otherwise reduce the biological activity of the TNF molecule. Similarly, the TNFR polypeptide, fragment or fusion protein may be chemically modified with one or more functional groups provided that such functional groups do not interfere with the ability of the TNFR-binding moiety to bind to TNF and sequester, inhibit, antagonise, abrogate or otherwise reduce its biological activity.

### Polynucleotides

The present disclosure further extends to polynucleotides that encode any of the TNFR polypeptides, TNF-binding fragments thereof and fusion proteins of the present invention, as herein described.

Polynucleotides that encode any of the TNFR polypeptides, TNF-binding fragments thereof and fusion proteins of the present invention can be readily prepared by a skilled person using techniques which are known in the art, such as those described in Sambrook et al. “Molecular Cloning”, A laboratory manual, cold Spring Harbor Laboratory Press, Volumes 1-3, 2001 (ISBN-0879695773), and Ausubel et al. Short Protocols in Molecular Biology. John Wiley and Sons, 4^{th} Edition, 1999 (ISBN-0471250929).

Polynucleotides that encode any of the TNFR polypeptides, TNF-binding fragments thereof and fusion proteins of the present invention may be provided as constructs in the form of a plasmid, vector, transcription or expression cassette which comprises at least one nucleic acid. The construct may be comprised within a recombinant host cell which comprises one or more constructs. Expression may conveniently be achieved by culturing, under appropriate conditions, recombinant host cells containing suitable nucleic acid sequences. Following expression, the TNFR polypeptides, fragments and fusion proteins according to the present invention, as described herein, may be isolated and/or purified using any suitable technique known to persons skilled in the art.

Systems for cloning and expression of polypeptides, fragments thereof and fusion proteins in a variety of different host cells are well known to persons skilled in the art. Examples of suitable host cells include bacteria, mammalian cells, yeast, insect and baculovirus systems. Mammalian cell lines available in the art for expression of a heterologous polypeptide include Chinese hamster ovary (CHO) cells, HeLa cells, baby hamster kidney cells and NSO mouse myeloma cells. A common, preferred bacterial host is E. coli. The expression of the TNFR polypeptides, fragments or fusion proteins according to the present invention, as described herein, in prokaryotic cells such as E. coli is also well established in the art. Expression in eukaryotic cells in culture is also available to those skilled in the art as an option for production of a polypeptide.

In an embodiment, the isolated polynucleotide encodes any of the equine or feline TNFR p80 polypeptides as herein described. In an embodiment, the polynucleotide comprises the nucleic acid sequence of SEQ ID NO:6. In another embodiment, the polynucleotide comprises the nucleic acid sequence of SEQ ID NO:42.

In an embodiment, the polynucleotide comprises a nucleic acid sequence encoding the TNFR polypeptide of the present invention, as herein described, operatively linked to a nucleic acid sequence encoding a TNFR signal sequence. As noted elsewhere herein, the inventors have, for the first time, identified the correct signal sequence for equine TNFR80, represented by the amino acid sequence MAPVAVWAALAVGLQLWAAGRA (SEQ ID NO:4). The inventors have, for the first time, also identified the correct signal sequence for feline TNFR80, represented by the amino acid sequence of SEQ ID NO:38 or SEQ ID NO:39. This allows, for the first time, the recombinant expression of an equine or feline TNFR p80 polypeptide or TNF-binding fragment thereof, or a fusion protein comprising said polypeptide or fragment. It does so by expressing the correct signal sequence that facilitates the transport and release of the mature and functional form of an equine or feline TNFR p80 polypeptide moiety from the host cell.

Thus, in an embodiment, the polynucleotide comprises a nucleic acid sequence encoding the TNFR polypeptide of the present invention, or a TNF-binding fragment thereof, as herein described, operatively linked to a nucleic acid sequence encoding a TNFR signal sequence comprising the amino acid sequence MAPVAVWAALAVGLQLWAAGRA (SEQ ID NO:4), or an amino acid sequence having at least 70%, 75%, 80%, 85%, 90%, 93%, 95%, 96%, 97%, 98% or 99% sequence similarity thereto after optimal alignment. In another embodiment, the polynucleotide comprises a nucleic acid sequence encoding the TNFR polypeptide of the present invention, or a TNF-binding fragment thereof, as herein described, operatively linked to a nucleic acid sequence encoding a TNFR signal sequence comprising an amino acid sequence having at least 70%, 75%, 80%, 85%, 90%, 93%, 95%, 96%, 97%, 98% or 99% sequence identity to SEQ ID NO:4 after optimal alignment. In another embodiment, the polynucleotide comprises a nucleic acid sequence encoding the TNFR polypeptide of the present invention, as herein described, operatively linked to a nucleic acid sequence encoding a TNFR signal sequence consisting of the amino acid sequence MAPVAVWAALAVGLQLWAAGRA (SEQ ID NO:4).

In some embodiments, the polynucleotide comprises a nucleic acid sequence encoding an equine TNFR p80 precursor molecule comprising the amino acid sequence of SEQ ID NO:5, or an amino acid sequence having at least 70%, 75%, 80%, 85%, 90%, 93%, 95%, 96%, 97%, 98% or 99% sequence similarity thereto after optimal alignment, wherein the TNFR precursor molecule is capable of being expressed in a host cell and, upon expression, the TNFR signal sequence is cleaved to generate a functional equine TNFR polypeptide, or a TNF-binding fragment thereof, having an N-terminal VPAQV motif (with the proviso that the TNFR polypeptide, or TNF-binding fragment thereof, does not have an N-terminal VPAQVVL motif). In another embodiment, the polynucleotide comprises a nucleic acid sequence encoding an equine TNFR p80 precursor molecule comprising an amino acid sequence having at least 70%, 75%, 80%, 85%, 90%, 93%, 95%, 96%, 97%, 98% or 99% sequence identity to SEQ ID NO:5 after optimal alignment, wherein the TNFR precursor molecule is capable of being expressed in a host cell and, upon expression, the TNFR signal sequence is cleaved to generate a functional equine TNFR polypeptide, or a TNF-binding fragment thereof, having an N-terminal VPAQV motif (with the proviso that the TNFR polypeptide, or TNF-binding fragment thereof, does not have an N-terminal VPAQVVL motif). In an embodiment, the TNFR polypeptide or fragment thereof comprises an N-terminal VPAQVVF motif. In an embodiment, the polynucleotide comprises a nucleic acid sequence that encodes an equine TNFR p80 precursor molecule that consists of an amino acid sequence of SEQ ID NO:5.

In another embodiment, the polynucleotide comprises a nucleic acid sequence encoding a fusion protein of the present invention, as herein described, operatively linked to a nucleic acid sequence encoding an equine TNFR p80 signal sequence comprising the amino acid sequence MAPVAVWAALAVGLQLWAAGRA (SEQ ID NO:4), or an amino acid sequence having at least 70%, 75%, 80%, 85%, 90%, 93%, 95%, 96%, 97%, 98% or 99% sequence similarity thereto after optimal alignment, wherein the nucleic acid sequence encodes a fusion protein comprising a TNFR precursor molecule that, upon expression in a host cell, is capable of being cleaved (at the TNFR signal sequence) to form a fusion protein having an N-terminal VPAQV motif (with the proviso that the fusion protein does not have an N-terminal VPAQVVL motif). In another embodiment, the polynucleotide comprises a nucleic acid sequence encoding a fusion protein of the present invention, as herein described, operatively linked to a nucleic acid sequence encoding an equine TNFR p80 signal sequence comprising an amino acid sequence having at least 70%, 75%, 80%, 85%, 90%, 93%, 95%, 96%, 97%, 98% or 99% sequence identity to SEQ ID NO:4 after optimal alignment, wherein the nucleic acid sequence encodes a fusion protein comprising a TNFR precursor molecule that, upon expression in a host cell, is capable of being cleaved (at the TNFR signal sequence) to form a fusion protein having an N-terminal VPAQV motif (with the proviso that the fusion protein does not have an N-terminal VPAQVVL motif). In an embodiment, the fusion protein comprises an N-terminal VPAQVVF motif. In another embodiment, the polynucleotide comprises a nucleic acid sequence encoding a fusion protein of the present invention, as herein described, operatively linked to a nucleic acid sequence encoding a TNFR signal sequence consisting of the amino acid sequence MAPVAVWAALAVGLQLWAAGRA (SEQ ID NO:4).

In another embodiment, the polynucleotide comprises a nucleic acid sequence encoding a fusion protein comprising the amino acid sequence of SEQ ID NO: 16 or SEQ ID NO:17, or an amino acid sequence having at least 70%, 75%, 80%, 85%, 90%, 93%, 95%, 96%, 97%, 98% or 99% sequence similarity to any of the foregoing after optimal alignment, wherein the fusion protein, upon expression in a host cell, is capable of being cleaved (at the TNFR signal sequence) to form a fusion protein that comprises an N-terminal VPAQV motif (with the proviso that the fusion protein does not have an N-terminal VPAQVVL motif). In another embodiment, the polynucleotide comprises a nucleic acid sequence encoding a fusion protein comprising an amino acid sequence having at least 70%, 75%, 80%, 85%, 90%, 93%, 95%, 96%, 97%, 98% or 99% sequence identity to SEQ ID NO:16 or SEQ ID NO:17 after optimal alignment, wherein the fusion protein, upon expression in a host cell, is capable of being cleaved (at the TNFR signal sequence) to form a fusion protein that comprises an N-terminal VPAQV motif (with the proviso that the fusion protein does not have an N-terminal VPAQVVL motif). In an embodiment, the fusion protein is capable of being cleaved to form a fusion protein comprising an N-terminal VPAQVVF motif.

In another embodiment, the polynucleotide comprises a nucleic acid sequence encoding a the amino acid sequence consisting of SEQ ID NO: 17 or SEQ ID NO:18.

Also disclosed herein is a recombinant vector comprising the polynucleotides as herein described. In an embodiment, the vector is under the control of, or is operably linked to, a promoter.

The terms “polynucleotide” and “nucleic acid” are used interchangeably to refer to polymeric forms of nucleotides of any length. The term polynucleotide also refers interchangeably to double and single stranded nucleic acid molecules.

The terms “operatively linked”, “operable linkage” and the like are understood to mean, for example, the sequential arrangement of a promoter with the nucleic acid sequence to be transcribed and, if appropriate, additional regulatory elements such as, for example, a terminator and/or a polyadenylation signal such that each of the regulatory elements can fulfil its function when the nucleic acid sequence is transcribed, depending on the arrangement of the nucleic acid sequences. It is to be understood that a direct linkage in the chemical sense is not necessarily required. Genetic control sequences such as, for example, enhancer sequences, may also exert their function on the target sequence in positions which are distal (i.e., further away) from other nucleic acid molecules. In some embodiments, the nucleic acid sequence to be transcribed is positioned behind the sequence acting as promoter, so that both sequences are covalently linked to one another.

Methods of Production and/or Purification

The present disclosure also extends to methods of producing and/or purifying any of the TNFR polypeptides, TNF-binding fragments thereof and fusion proteins of the present invention, as herein described. Suitable methods of production and purification would be known to persons skilled in the art.

In an illustrative example, the TNFR polypeptides, TNF-binding fragments and fusion proteins of the present invention, as herein described, can be produced using recombinant DNA technology. By identifying, for the first time, the correct signal sequence of the native equine and feline TNFR p80 isoforms, enabled herein are methods by which recombinant TNFR polypeptides, TNF-binding fragments thereof and recombinant fusion proteins, as herein described can be expressed and produced by a host cell by recombinant DNA technology. This is facilitated, at least in part, by the correctly identified TNFR signal sequence of the equine and feline TNFR p80 isoforms. This is to be contrasted with the predicted equine and feline TNFR p80 isoforms, represented by SEQ ID NO:19 (equine; XM_00507617) and SEQ ID NO:64 (feline; XP_003989632.1), which lack the correct signal sequence and are therefore unable to generate a functional TNFR polypeptides.

Suitable host cells would be known to persons skilled in the art. Illustrative examples include eukaryotic cells and prokaryotic cells. In some embodiments, the recombinant vector is the vector of the present invention, as herein described. In some embodiments, the polynucleotide is the polynucleotide of the present invention, as herein described.

In a yet further aspect there is provided a host cell line, or a derivative or progeny cell thereof that produces the TNFR polypeptides, TNF-binding fragments thereof, or the fusion proteins according to the present invention, as herein described.

In another aspect of the present invention, there is provided a method for purification of the fusion proteins of the present invention, as herein described. The inventors have previously identified that Protein A chromatography can be a useful tool in the purification of HC2, but not HC6, isotypes of equine immunoglobulins. Thus, equine-derived immunoglobulin heavy chain domains of type IgG2 are purified more efficiently than those of type IgG6. Accordingly, purification of fusion proteins according to an embodiment of the present invention provides higher yields where the fusion partner comprises an amino acid sequence derived from the immunoglobulin heavy chain constant domain of equine IgG2, as compared to equine IgG6. This is an advantage in that often protein purification based on Protein A matrices is used to obtain a commercially relevant yield of a protein for therapeutic use. Accordingly, the use of Protein A purification for the purification of a fusion protein according to an embodiment of the present invention provides higher yields where the immunoglobulin heavy chain constant region is derived from equine IgG2. This feature, coupled to the surprising observation that the resulting purified proteins do not recruit complement or mediate downstream effector functions when administered to an equine, provides compositions according to the invention which can be advantageously administered to equines for therapeutic purposes and, in particular, protein therapeutics which are surprisingly advantageous over fusion proteins comprising polypeptides of human origin.

Thus, in an aspect of the present invention, there is provided a method of purifying a dimer form of the fusion protein of the present invention, as herein described, the method comprising a step of eluting the dimer form of the fusion protein from Protein A at a pH of 4.7 to 5.3. In an embodiment, the dimer form of the fusion protein is eluted from Protein A at a pH of 4.8 to 5.2. In another embodiment, the fusion protein comprises a CH2 domain and/or a CH3 domain of an equine IgG2 immunoglobulin heavy chain constant region, or a fragment thereof.

A further aspect of the present invention provides for the selective purification of a preferred dimer form of the fusion protein of the present invention, in particular, fusion proteins comprising an immunoglobulin heavy chain constant region, or portion thereof (e.g., having a CH2 and/or CH3 domain) of equine IgG2, from higher molecular weight multimers formed by host cell expression by elution at optimal pH from a Protein A column.

The above methods may be applied as a modification of standard methods of purification by Protein A chromatography to purify the desired TNFR fusion protein dimer from aggregated forms thereof. This advantageously yields a product of higher specific activity and purity and, furthermore, the removal of aggregates reduces the potential for immunogenicity. Standard procedures for Protein A purification of immunoglobulins are known to persons skilled in the art and typically include binding at neutral pH and elution at low pH.

In an embodiment, the process of producing the fusion protein of the present invention results in the presentation of the TNFR extracellular domain which, based on the inventors' analysis, will retain the conformation of the receptor, maintain binding specificity and avidity and increase the receptor size above that eliminated in the kidney, while reducing the presence of immunogenic epitopes which may result in neutralising antibodies (particularly xenoantibodies) being generated against the receptor if it were to be administered to a target host in an unaltered form.

The TNFR polypeptides or fusion proteins according to the present invention, as herein described, including TNF-binding fragments thereof, may be produced wholly or partly by chemical synthesis. For example, the TNFR polypeptides, TNF-binding fragments thereof and fusion polypeptides can be prepared by techniques which are well known to the person skilled in the art, such as standard liquid peptide synthesis or by solid-phase peptide synthesis methods. Alternatively, the TNFR polypeptides, fragments and fusion proteins according to the present invention, as herein described, may be prepared in solution using liquid phase peptide synthesis techniques, or further by a combination of solid-phase, liquid phase and solution chemistry.

In another aspect of the present invention, there is provided a method for producing a fusion protein comprising a TNFR polypeptide operatively linked to a polypeptide comprising a CH2 domain and a CH3 domain of an equine or feline IgG immunoglobulin, the method comprising:
(i) providing a host cell comprising a recombinant vector that is capable of expressing a polynucleotide encoding the fusion protein according to the present invention;
(ii) culturing the host cell under conditions suitable for expression of the fusion protein, and
(iii) recovering the fusion protein.

The term “isolated”, when used in reference to the TNFR polypeptides, TNF-binding fragments or fusion proteins according to the present invention, as herein described, or polynucleotides which encode the same, as herein described, refers to the state in which said polypeptides, fragments, proteins and polynucleotides are provided in an isolated and/or purified form; that is, they have been separated, isolated or purified from their natural environment, and are provided in a substantially pure or homogeneous form, or, in the case of nucleic acids, free or substantially free of nucleic acid or genes of origin other than the sequence encoding a polypeptide with the required function. Accordingly, such isolated polypeptides, fragments, proteins and polynucleotides will be free or substantially free of material with which they are naturally associated, such as other polypeptides or nucleic acids with which they are found in their natural environment, or the environment in which they are prepared (e.g. cell culture) when such preparation is by recombinant DNA technology practised in vitro or in vivo.

### Pharmaceutical Compositions and Kits

The present disclosure also extends to pharmaceutical compositions comprising any of the TNFR polypeptides, TNF-binding fragments and fusion proteins of the present invention, as herein described. Thus, in another aspect disclosed herein, there is provided a pharmaceutical composition comprising the TNFR polypeptide, TNF-binding fragment thereof or fusion protein of the present invention, as herein described, and a pharmaceutically acceptable carrier or excipient. Also disclosed herein is a pharmaceutical composition comprising a polynucleotide encoding a TNFR polypeptide, TNF-binding fragment thereof or fusion protein according to the present invention, as herein described, and a pharmaceutically acceptable carrier or excipient.

In an embodiment, the pharmaceutical composition further comprises at least one further TNF antagonist and/or an anti-inflammatory compound. In an embodiment, the TNF antagonist is methotrexate. In some embodiments, the pharmaceutical composition further comprises at least one analgesic, NSAID, opioid, corticosteroid, steroid or antagonist of nerve growth factor.

The TNFR polypeptides, TNF-binding fragments thereof, fusion proteins or polynucleotides according to the present invention, as described herein, may be formulated with diluents or adjuvants and still, for practical purposes, be considered as being provided in an isolated form. For example, the TNFR polypeptides, fragments, fusion proteins or polynucleotides, can be mixed with gelatin or other carriers if used to coat microtitre plates for use in immunoassays, or can be mixed with pharmaceutically acceptable carriers or diluents when used in diagnosis or therapy. The TNFR polypeptides, fragments and/or fusion proteins may be glycosylated, either naturally or by systems of heterologous eukaryotic cells (e.g. CHO or NSO cells), or they may be (e.g., if produced by expression in a prokaryotic cell) unglycosylated.

The present invention also extends to heterogeneous preparations comprising the TNFR polypeptides, fragments, fusion proteins or polynucleotides according to the present invention, as described herein. In some embodiments, such preparations may be mixtures of fusion proteins with TNFR polypeptides lacking a fusion partner, with various degrees of glycosylation and/or with derivatised amino acids, such as cyclization of an N-terminal glutamic acid to form a pyroglutamic acid residue.

In some embodiments, the pharmaceutical composition of the present invention is formulated in a liquid formulation, a lyophilized formulation, a lyophilized formulation that is reconstituted as a liquid, or as an aerosol formulation. In an embodiment, the TNFR polypeptides, fragments and/or the fusion proteins according to the present invention, as described herein, are present in the composition at a concentration of about 0.5 mg/ml to about 250 mg/ml, about 0.5 mg/ml to about 45 mg/ml, about 0.5 mg/ml to about 100 mg/ml, about 100 mg/ml to about 200 mg/ml or about 50 mg/ml to about 250 mg/ml.

In some embodiments, the composition further comprises a buffer. Typically, the pH of the composition is from about pH 5.5 to about pH 6.5. In some embodiments, the buffer may comprise from about 4 mM to about 60 mM histidine buffer, about 5 mM to about 25 mM succinate buffer, or about 5 mM to 25 mM acetate buffer. In some embodiments, the buffer comprises sodium chloride at a concentration of from about 10 mM to 300 mM, typically at around 125 mM concentration and sodium citrate at a concentration of from about 5 mM to 50 mM, typically 25 mM. In some embodiments, the composition further comprises a surfactant, for example, at a concentration of just above 0% to about 0.2%. Suitable surfactants would be known to persons skilled in the art. Illustrative examples include polysorbate-20, polysorbate-40, polysorbate-60, polysorbate-65, polysorbate-80, polysorbate-85, or combinations thereof. In an embodiment, the surfactant is polysorbate-20 and may further comprise sodium chloride at a concentration of about 125 mM and sodium citrate at a concentration of about 25 mM.

The TNFR polypeptides, fragments, fusion proteins and/or polynucleotides according to the present invention, as herein described, may be administered alone. In some embodiments, they will be administered as a pharmaceutical composition which will generally comprise a suitable pharmaceutically acceptable excipient, diluent or carrier selected, for example, depending on the intended route of administration. Suitable pharmaceutical carriers are known to persons skilled in the art. Illustrative examples include water, glycerol, ethanol and the like.

The TNFR polypeptides, fragments, fusion proteins and/or polynucleotides according to the present invention, as herein described, may be administered to a subject (i.e., an equine or feline) in need thereof exogenously or via any other suitable route of administration. Typically, the composition can be administered parenterally by injection or infusion. Suitable routes of parenteral administration would be known to persons skilled in the art. Illustrative examples include intravenous, intraperitoneal, intramuscular, subcutaneous or transmucosal. Routes of administration may further include topical and enteral, for example, mucosal (including pulmonary), oral, nasal and rectal.

In embodiments where the pharmaceutical composition of the present invention is to be administered as an injectable composition, for example in intravenous, intradermal or subcutaneous application, the active ingredient can be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection or, Lactated Ringer's injection. Preservatives, stabilisers, buffers, antioxidants and/or other additives may also be included, as required. The pharmaceutical compositions may also be administered via microspheres, liposomes, other microparticulate delivery systems or sustained release formulations placed in certain tissues, including blood.

Examples of the techniques and protocols mentioned above and other techniques and protocols which may be used in accordance with the present invention can be found in Remington's Pharmaceutical Sciences, 18th edition, Gennaro, A. R., Lippincott Williams & Wilkins; 20th edition ISBN 0-912734-04-3 and Pharmaceutical Dosage Forms and Drug Delivery Systems; Ansel, H. C. et al. 7th Edition ISBN 0-683305-72-7.

In another aspect of the present invention, there is provided a kit comprising the TNFR polypeptides, fragments, fusion proteins and/or polynucleotides according to the present invention, as herein described. Such kits may be used for the treatment and/or prevention of a condition mediated by TNF and may therefore further comprise instructions for such use.

### Methods and Uses for Treatment or Prophylaxis

As noted elsewhere herein, the present disclosure is predicated on the inventor's surprising finding of the correct amino acid sequences of the extracellular domain of equine and feline TNFR p80 isoforms. Accordingly, the TNFR p80 polypeptides, TNF-binding-fragments thereof and fusion proteins comprising any of the foregoing, as herein described, can be employed as a therapeutic or prophylactic agent to bind to and sequester, inhibit, abrogate, antagonize or otherwise block the biological activity of TNF in an equine or feline subject. Moreover, by virtue of having an N-terminal amino acid sequence that is native to the target species (i.e., equine or feline), the immunogenicity that would otherwise be associated with the use of a TNFR p80 polypeptide or TNF-binding-fragment thereof having an N-terminal amino acid sequence that is foreign to the target species, can be avoided or minimised.

Thus, in an aspect disclosed herein, there is provided a TNFR polypeptide, or a TNF-binding fragment thereof, or the fusion protein, as herein described, for use in the treatment or prevention of a condition mediated by TNF in a feline. In another aspect disclosed herein, there is provided a TNFR polypeptide, or a TNF-binding fragment thereof, or the fusion protein, as herein described, for use in the treatment or prevention of a condition mediated by TNF in an equine.

In yet another aspect disclosed herein, there is provided use of a TNFR polypeptide, or a TNF-binding fragment thereof, or the fusion protein, as herein described, in the preparation of a medicament for the treatment or prevention of a condition mediated by TNF in a feline. In a further aspect disclosed herein, there is provided use of a TNFR polypeptide, or a TNF-binding fragment thereof, or the fusion protein, as herein described, in the preparation of a medicament for the treatment or prevention of a condition mediated by TNF in an equine.

In yet another aspect disclosed herein, there is provided a method for treating or preventing a condition mediated by TNF in an equine, the method comprising the step of administering a therapeutically effective amount of use of a TNFR polypeptide, or a TNF-binding fragment thereof, or the fusion protein, as herein described, to an equine in need thereof. In a further aspect disclosed herein, there is provided a method for treating or preventing a condition mediated by TNF in a feline, the method comprising the step of administering a therapeutically effective amount of use of a TNFR polypeptide, or a TNF-binding fragment thereof, or the fusion protein, as herein described, to a feline in need thereof.

Conditions mediated by TNF in an equine or feline subject would be known to persons skilled in the art. Illustrative examples include an inflammatory mediated condition, a chronic inflammatory disease, arthritis, such as immune mediated polyarthritis, rheumatoid arthritis, osteoarthritis, polyarthritidies, juvenile idiopathic arthritis, psoriatic arthritis, ankylosing spondylitis, Crohn's disease, ulcerative colitis, psoriasis, systemic vasculitis, atopic dermatitis, congestive heart failure, refractory uveitis, bronchial asthma, allergic conditions, sepsis, shock, diabetes mellitus, and neuro-degenerative conditions, such as Alzheimer's disease, Parkinson's disease, stroke, amyotrophic lateral sclerosis, Behcet's disease, bullous dermatitis, neutrophilic dermatitis, toxic epidermal necrolysis, systemic vasculitis, pyoderma gangrenosum, pustular dermatitis, cerebral malaria, hemolytic uremic syndrome, pre-eclampsia, allograft rejection, otitis media, snakebite, erythema nodosum, myelodysplastic syndromes, graft versus host disease, dermatomyositis and polymyositis.

In an embodiment disclosed herein, the condition mediated by TNF is selected from the group consisting of an inflammatory mediated condition, a chronic inflammatory disease, arthritis, such as immune mediated polyarthritis, rheumatoid arthritis, osteoarthritis, polyarthritidies, juvenile idiopathic arthritis, psoriatic arthritis, ankylosing spondylitis, Crohn's disease, ulcerative colitis, psoriasis, systemic vasculitis, atopic dermatitis, congestive heart failure, refractory uveitis, bronchial asthma, allergic conditions, sepsis, shock, diabetes mellitus, and neuro-degenerative conditions, such as Alzheimer's disease, Parkinson's disease, stroke and amyotrophic lateral sclerosis.

In an embodiment, the condition mediated by TNF is arthritis or an arthritic condition, including a symptom thereof. In certain embodiments, the arthritis or arthritic condition includes the condition selected from the group consisting of immune mediated polyarthritis, rheumatoid arthritis, osteoarthritis, psoriatic arthritis, juvenile idiopathic arthritis, ankylosing spondylitis and related conditions. In some embodiments, the treatment of the arthritis or arthritic condition comprises ameliorating, inhibiting, reducing or suppressing the immune response which is causative, associated with, or attributable to the arthritic condition.

In some embodiments, the TNFR polypeptides, TNF-binding fragments or fusion proteins of the present invention, as herein described, will bind with high affinity binding to equine or feline TNF, thereby neutralising its biological function (activity). As stated elsewhere herein, and without being bound by theory or by any particular mode of action, the TNFR polypeptides, TNF-binding fragments and fusion proteins of the present invention, as herein described, inhibit the binding of TNF to TNFR cell membrane-associated receptors.

With respect to fusion proteins, as noted elsewhere herein, it may be desirable to use a fusion partner that is immunologically inert (e.g., comprising only amino acid residues present in the target species), such that, when administered to the target (equine or feline), immunogenicity against the fusion protein is minimised or avoided. Thus, the TNFR polypeptides, TNF-binding fragments thereof and fusion proteins according to the present invention, as herein described, are particularly suitable for long-term administration (e.g., as part of a multiple dosage regimen) for the treatment or prophylaxis of conditions mediated, at least in part, by TNF in the target species.

As used herein, the terms “effective amount” and “therapeutically effective amount” are used interchangeably to mean an amount of TNFR polypeptide, TNF-binding fragment thereof and/or fusion protein, as herein described, which is required to inhibit, antagonise, abrogate or otherwise block TNF binding to endogenous, cell bound TNF receptors in vivo and, hence, inhibit, antagonise, abrogate or otherwise block the biological activity of TNF in vivo. The terms “effective amount” and “therapeutically effective amount” also mean an amount of TNFR polypeptide, TNF-binding fragment thereof and/or fusion protein, as herein described, that is sufficient to effect a beneficial or desired clinical results. An “effective amount” may therefore be an amount that achieves one or more of the following: a reduction in TNF levels, a reduction of an inflammatory response or a reduction, prevention or amelioration of a TNF-mediated disease or condition, or a symptom thereof. An effective amount need not be achieved by a single dose or administration, but may be achieved by administering the TNFR polypeptide, TNF-binding fragment thereof and/or fusion protein, as herein described, in more that one dose or administration. Split dosing may also be utilized where the total daily amount of the extract is given over a period of from 1 to 24 hours in different or evenly proportional amounts. For example, two half doses every 12 hours.\

Examples of dosage regimens which can be administered to an equine or feline subject can be selected from the group comprising, but not limited to, 1 μg/kg/day through to 20 mg/kg/day, 1 μg/kg/day through to 10 mg/kg/day and 10 μg/kg/day through to 1 mg/kg/day. In some embodiments, the dosage will be such that a plasma concentration of from 1 μg/ml to 100 μg/ml of the antibody is obtained. However, persons skilled in the art will understand that the actual dose or doses to be administered, and rate and time-course of administration, will depend on the nature and severity of the condition being treated, the age, sex and weight of the equine or feline subject being treated, as well as the route of administration. Further consideration may be given to the properties of the composition, for example, its binding activity and in vivo plasma life, the concentration of the TNFR polypeptide, TNF-binding fragment and/or fusion protein of present invention, as described herein, or polynucleotides encoding same, in the formulation. Prescription of treatment, e.g. decisions on dosage etc., is ultimately within the responsibility and at the discretion of veterinary practitioners and other veterinary doctors, and typically takes account of the disorder to be treated, the condition of the equine subject in question, the site of delivery, the method of administration and other factors known to practitioners, some of which are described herein.

The present invention also extends to combination therapy. For example, in an embodiment, the method further comprises the step of administering to an equine or feline subject in need thereof, simultaneously or sequentially, at least one further agent which may enhance and/or supplement the effectiveness of the TNFR polypeptide, TNF-binding fragment thereof or fusion protein of the present invention, as herein described. In some embodiments, the foregoing uses and methods of the present invention further comprise the step of administering to an equine or feline subject in need thereof, simultaneously or sequentially, at least one additional TNF inhibitory agent (TNF antagonist) and/or anti-inflammatory agent. Said additional agent may comprise a drug useful for treating a chronic inflammatory condition, in particular, a TNF-mediated condition. In some embodiments, the additional agent may be a TNF antagonist, such as methotrexate, an analgesic, a compound which is a cytokine suppressing anti-inflammatory drug, an NSAID, an opioid, a corticosteroid or a steroid.

Suitable analgesics would be known to persons skilled in the art. Illustrative examples include butorphanol, buprenorphine, fentanyl, flunixin meglumine, merpidine, morphine, nalbuphine and derivatives thereof. Suitable NSAIDS include, but are not limited to, acetaminophen, acetylsalicylic acid, carprofen, etodolac, ketoprofen, meloxicam, firocoxib, robenacoxib, deracoxib and the like.

In some embodiments, the at least one further agent may be a therapeutically active agent selected from the group consisting of an antibiotic, an antifungal agent, an antiprotozoal agent, an antiviral agent and similar therapeutic agents. In some embodiments, the at least one further agent may be an inhibitor of mediator(s) of inflammation, such as a PGE-receptor antagonist, an immunosuppressive agent, such as cyclosporine, or anti-inflammatory glucocorticoids. In some embodiments, the at least one further agent may be an agent which is used for the treatment of cognitive dysfunction or impairment, such as memory loss or related conditions which may become increasingly prevalent in older equines. Further still, the at least one further agent may be an anti-hypertensive or other compound used for the treatment of cardiovascular dysfunction, for example, to treat hypertension, myocardial ischemia, congestive heart failure and the like. Further still, the at least one further agent may be a diuretic, vasodilator, beta-adrenergic receptor antagonist, angiotensin-II converting enzyme inhibitor, calcium channel blocker or HMG-CoA reductase inhibitor.

The TNFR polypeptides, TNF-binding fragments thereof and the fusion proteins of the present invention, as herein described, are typically administered exogenously, for example, by intravenous or subcutaneous administration. However, in certain embodiments, a vector may be used to deliver a polynucleotide into a cell of the target, the polynucleotide encoding a TNFR polypeptide, fragment or fusion protein, as described elsewhere herein.

As noted elsewhere herein, dosage regimens can include a single administration of the TNFR polypeptides, TNF-binding fragments, fusion proteins or polynucleotides according to the present invention, as described herein, or multiple administrative doses, as required. The TNFR polypeptides, TNF-binding fragments, fusion proteins or polynucleotides according to the present invention, as described herein, can be administered sequentially, simultaneously or separately, with other anti-inflammatory agents or TNF antagonists. In some embodiments, the TNFR polypeptides or TNF-binding fragments thereof can be administered sequentially, simultaneously or separately, with a fusion protein according to the present invention, as described herein.

### Agents and Methods for Treatment and Diagnosis

From the knowledge of the correct amino acid sequence of the equine and feline TNFR p80 isoforms, as herein described, agents can be developed that bind to an equine or feline TNFR p80 isoform. Thus, also provided herein is an agent capable of binding specifically to any of the TNFR polypeptides, TNF-binding fragments therefore or fusion proteins, as herein described.

In an embodiment, the agent is an immuno-interactive molecule. An immuno-interactive molecule, in the context of the present disclosure, is a molecule capable of binding specifically to an equine or feline TNFR p80 polypeptide, as herein described, in vitro or in vivo. In an embodiment, the agent specifically binds to an epitope located at the N-terminus of the TNFR polypeptide, wherein the epitope comprises an N-terminal VPAQV motif. In an embodiment, the TNFR polypeptide is an equine TNFR p80 isoform and the epitope to which the agent specifically binds comprises an N-terminal VPAQVAF motif. In another embodiment, the TNFR polypeptide is a feline TNFR p80 isoform and the epitope to which the agent specifically binds comprises an N-terminal VPAQVVL motif.

Suitable agents would be known to persons skilled in the art. Illustrative examples include an antibody or a fragment thereof that is capable of binding specifically to the TNFR p80 polypeptide, fragment or fusion protein. Antibodies suitable for use in accordance with the methods disclosed herein would be known to those skilled in the art. Illustrative examples include polyclonal, monoclonal, mono-specific, poly-specific (including bi-specific), humanized, single-chain, chimeric, synthetic, recombinant, hybrid, mutated, and CDR-grafted antibodies. Various techniques for producing antibodies and binding fragments thereof are known in the art. Antibodies may be derived from any species, including but not limited to, rat, mouse, goat, guinea pig donkey, rabbit, horse, lama, camel, or any avian species (e.g., chicken, duck). The antibody may be of any suitable isotype, such as IgG, IgM, IgA, IgD, IgE or any subclass thereof. The skilled addressee will appreciate that antibodies produced recombinantly, or by other means, for use in accordance with the methods embodied herein include fragments that are still capable of binding to or otherwise recognizing the TNFR p80 isoforms herein described. Examples include Fab, an F(ab)₂, Fv, scFv fragments. In an embodiment, the antibody is a monoclonal antibody or a TNFR p80-binding fragment thereof. The monoclonal antibody can be a humanised or deimmunised form of a non-human antibody.

In another embodiment disclosed herein, the agent is a multi-specific antibody that is capable of specifically binding to at least two epitopes, wherein one of the at least two antigens is an epitope of an equine or feline TNFR p80 polypeptide. The multi-specific antibody may a bi-specific antibody.

In an embodiment, the agent is capable of binding specifically to a cell surface-bound TNFR p80 polypeptide.

In another embodiment disclosed herein, the agent is a cytotoxic agent for targeting a cell that expresses an equine or feline TNFR p80 polypeptide. In this context, the agent may be labeled with a cytotoxic moiety. Suitable cytotoxic moieties are known to those skilled in the art, illustrative examples of which include a toxin, an apoptotic agent or a radioactive isotope. In another embodiment, the agent is an antibody that is cytotoxic to the cell by complement-directed means.

Also enabled herein is a method for treating or preventing a condition associated with aberrant TNFR expression in a feline subject, the method comprising the step of administering a therapeutically effective amount of an agent capable of binding specifically to a feline TNFR p80 polypeptide, as herein described, to a feline in need thereof. Also enabled herein is a method for treating or preventing a condition associated with aberrant TNFR expression in an equine subject, the method comprising the step of administering a therapeutically effective amount of an agent capable of binding specifically to an equine TNFR p80 polypeptide, as herein described, to an equine in need thereof.

The term “aberrant” typically means a level of expression of TNFR p80, as determined, for example, by (i) the concentration of the TNFR p80 polypeptide in a biological sample, (ii) the number of cells in a subject that express the TNFR p80 polypeptide and/or (iii) the level of expression of the TNFR p80 polypeptide on the surface of cells, where the level of expression of TNFR p80 correlates or is associated with an undesirable pathological or physiological condition or state, including a symptom thereof. The level of expression of a TNFR p80 polypeptide can be determined by any means known to persons skilled in the art, illustrative examples of which include Western blot analysis, enzyme-linked immunosorbent assay (ELISA) and fluorescence-activated cell sorting (FACS) analysis.

Also enabled herein is use of an agent that is capable of binding specifically to an equine or feline TNFR p80 isoform, as herein described, in the manufacture of a medicament for the treatment of a condition associated with aberrant TNFR p80 expression.

Also enabled herein is a method of diagnosing or monitoring in a subject a condition associated with aberrant expression of TNFR p80, the method comprising executing the step of analyzing a biological sample from a subject for the presence and/or level of TNFR p80, wherein the execution step comprises contacting the biological sample with an agent that is capable of binding specifically to a TNFR p80 polypeptide, as herein described, wherein the binding of the agent to a TNFR p80 polypeptide is indicative of presence of a cell that expresses the TNFR p80 polypeptide. Suitable agents that are capable of binding specifically to a TNFR p80 polypeptide (e.g., an equine or feline TNFR p80 polypeptide, as herein described) could be developed by persons skilled in the art armed with the knowledge of the correct amino acid sequences, as herein described. Illustrative examples are described elsewhere herein.

In an embodiment disclosed herein, the agent capable of binding specifically to a TNFR p80 polypeptide, as herein described, can be labelled with a detectable label to facilitate the detection of binding of the agent to the TNFR p80 polypeptide. Suitable detectable labels are known to those skilled in the art, an illustrative example of which includes any molecule that may be detected directly or indirectly so as to reveal the presence of a target (e.g., TNFR p80). Suitable examples of detectable labels which may be used in accordance with the present invention include fluorophores, radioactive isotopes, chromophores, electrochemiluminescent labels, bioluminescent labels, polymers, polymer particles, beads or other solid surfaces, gold or other metal particles or heavy atoms, spin labels, haptens, myc, nitrotyrosine, biotin and avidin. Others include phosphor particles, doped particles, nanocrystals or quantum dots. In an embodiment, a direct detectable label is used. Direct detectable labels may be detected per se without the need for additional molecules. In another embodiment, an indirect detectable label is used, which requires the employment of one or more additional molecules so as to form a detectable molecular complex (e.g., a biotin-avidin complex).

Also disclosed herein is a method of diagnosing or monitoring in a subject a condition mediated at least in part by TNF, the method comprising executing the step of analyzing a biological sample from a subject for the presence and/or level of TNF, wherein the execution step comprises contacting the biological sample with a TNFR polypeptide, TNF-binding fragment thereof or fusion protein, as herein described, wherein the binding of the polypeptide, binding fragment or fusion protein to TNF is indicative of presence of TNF in the sample.

In an embodiment disclosed herein, the TNFR polypeptide, TNF-binding fragment thereof or fusion protein, as herein described, can be labelled with a detectable label to facilitate the detection of binding of the polypeptide, binding fragment or fusion protein to TNF. Suitable detectable labels are known to those skilled in the art, an illustrative example of which includes any molecule that may be detected directly or indirectly so as to reveal the presence of a target (e.g., TNF). Suitable examples of detectable labels which may be used in accordance with the present invention include fluorophores, radioactive isotopes, chromophores, electrochemiluminescent labels, bioluminescent labels, polymers, polymer particles, beads or other solid surfaces, gold or other metal particles or heavy atoms, spin labels, haptens, myc, nitrotyrosine, biotin and avidin. Others include phosphor particles, doped particles, nanocrystals or quantum dots. In an embodiment, a direct detectable label is used. Direct detectable labels may be detected per se without the need for additional molecules. In another embodiment, an indirect detectable label is used, which requires the employment of one or more additional molecules so as to form a detectable molecular complex (e.g., a biotin-avidin complex).

The skilled person would understand that, where necessary, the diagnostic and/or monitoring methods disclosed herein may comprise using a secondary binding agent to increase the sensitivity of the method. As used herein, the term “secondary binding agent” means any substance that is capable of binding to or otherwise recognizing the TNFR p80 polypeptide, TNF-binding fragment thereof or fusion protein, as herein described, or any substance that is capable of binding to or otherwise recognizing the agent that is bound to TNF. Suitable secondary binding agents would be known to those skilled in the art. Illustrative examples include antibodies, or antigen binding fragments thereof, also referred to herein as secondary antibodies. Antibodies suitable for use as secondary binding agents would be known to those skilled in the art and include polyclonal, monoclonal, humanized, single-chain, chimeric, synthetic, recombinant, hybrid, mutated and CDR-grafted antibodies. Antibodies may be derived from any species, as hereinbefore described, and may be of any suitable isotype, such as IgG, IgM, IgA, IgD, IgE or any subclass thereof. The skilled addressee will appreciate that antibodies produced recombinantly, or by other means, for use in accordance with the present invention include antigen-binding fragments thereof that can still bind to or otherwise recognize the primary binding agent. Illustrative examples include Fab, F(ab)₂, Fv and scFv fragments.

The terms “recognize”, “recognizing” and the like, as used herein, mean an event in which one substance, such as a binding agent, directly or indirectly interacts with a target molecule in such a way that the interaction with the target may be detected. In some examples, a binding agent may react with a target, or directly bind to a target, or indirectly react with or bind to a target by directly binding to another substance that in turn directly binds to or reacts with a target. The terms “specific for”, “specifically” and the like, as used herein in the context of describing binding between two or more entities, mean that the binding is through a specific interaction between complementary binding partners, rather than through non-specific aggregation or association.

The diagnostic methods described herein may also be used to monitor the severity of a condition mediated, at least in part, by TNF or a condition associated with aberrant expression of TNFR p80. The diagnostic methods described herein may also be used to monitor the efficacy of a treatment regimen for a condition mediated, at least in part, by TNF or a condition associated with aberrant expression of TNFR p80. For example, samples may be obtained from a subject during the course of the condition (e.g., at daily, weekly, monthly, etc., intervals) and each sample analysed by the methods disclosed herein for the presence and/or level of expression of TNF and/or TNFR p80. Changes in the level of expression of TNF and/or TNFR p80, including the presence or absence of expression, is typically indicative of the severity of the condition. By following the subject over a period of time, the change in severity of the condition can therefore be monitored over time and, where necessary, a treatment regimen may be prescribed or modified in an attempt to compensate for the change in severity. In another illustrative example, samples can be obtained from a subject before and/or subsequent to the commencement of a treatment regimen and analysed by the methods disclosed herein for the presence and/or level of expression of TNF and/or TNFR p80. Changes in the level of expression of TNF and/or TNFR p80, including the presence or absence of expression, is typically indicative of the severity of the condition, as hereinbefore described. By following the subject over a period of time, the change in severity of the condition can therefore be monitored over time and, where necessary, the treatment regimen may be modified in an attempt to compensate for the change in severity (e.g., increasing the dosage of the treatment regimen).

The present invention will now be described with reference to the following examples which are provided for the purpose of illustration and are not intended to be construed as being limiting on the present invention. The methods and techniques of the present invention are generally performed according to conventional methods well known in the art and as described in various general and more specific references that are cited and discussed throughout the present specification unless otherwise indicated.

### EXAMPLES

### Example 1—Equine TNFR p80 Polypeptide

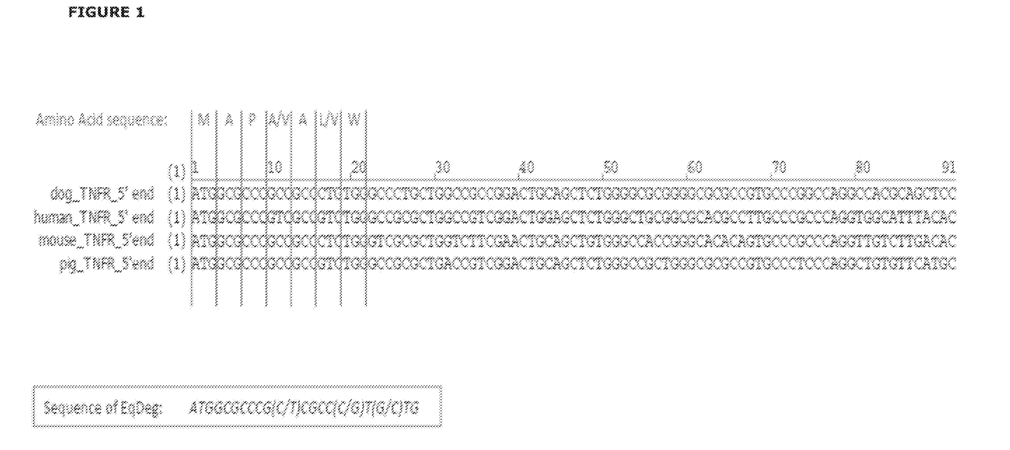

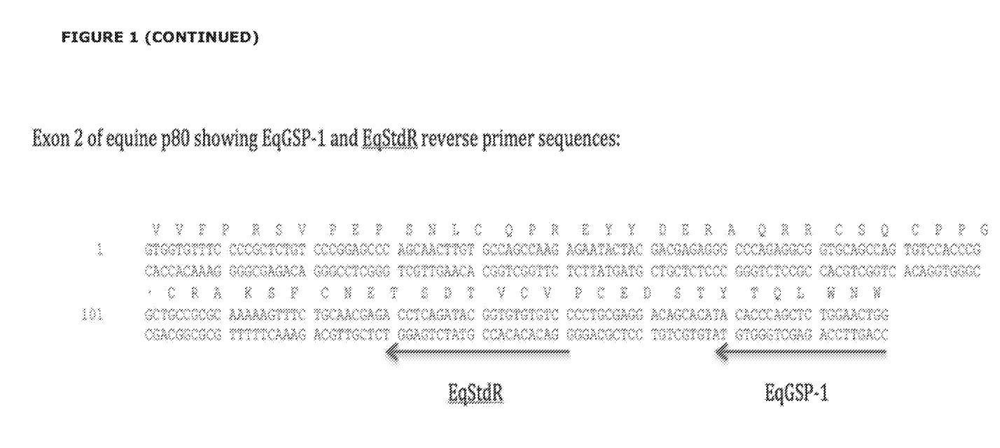

As described elsewhere herein, the inventors identified that the current predicted amino acid sequence of equine TNFR p80 (XM_005607617), comprises an incorrect leader sequence which would not function as a signal peptide. In order to identify the correct leader sequence for the p80 isoform of equine TNFR, a degenerate primer EqDeg was designed from knowledge of the DNA sequences that encode the human, mouse, pig and canine-terminal sequences of the p80 isoform of TNFR. FIG. 1 shows the alignment of canine, human, murine (mouse) and porcine (pig) p80 TNFR nucleotide sequences used for the design of a sense primer, EqDeg.

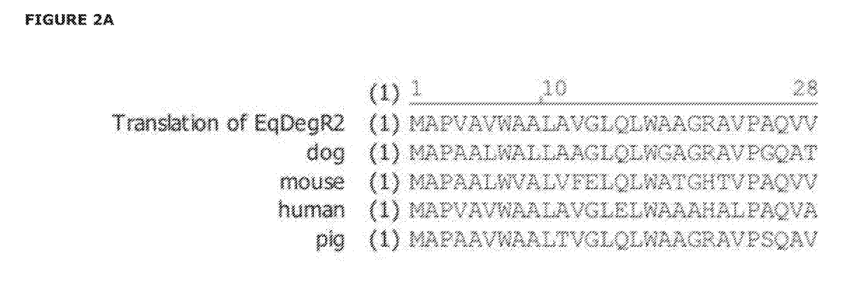

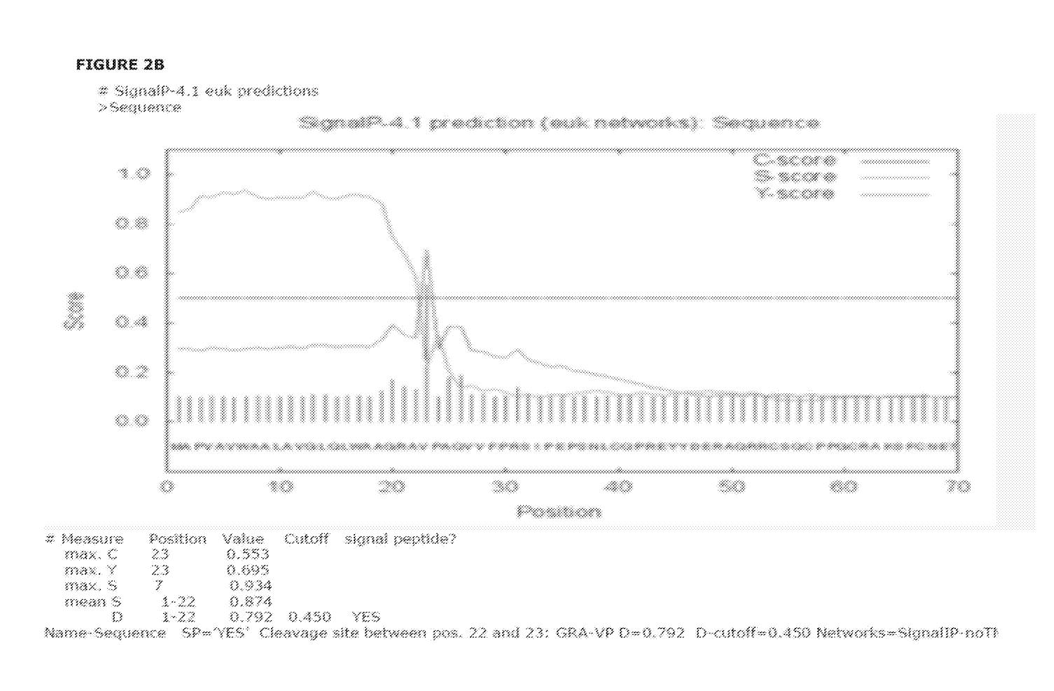

Total RNA was isolated from buffy coat cells prepared from 50 mls of equine blood. Specifically-primed first strand cDNA was prepared from 2 μg of the equine total RNA; the primer EqGSP-1 (see FIG. 1) was used for priming first strand synthesis. This specifically-primed first strand cDNA was then used as a template for standard PCR amplification using the degenerate forward primer EqDeg, and a nested reverse primer EqStdR. A 230 bp PCR product was obtained and this was subcloned and sequenced. An amino acid alignment of the translated sequenced PCR product, EqDegR2, is shown in FIG. 2.

The alignment data shown in FIG. 2A confirms that the correct coding sequence for equine p80 TNFR has been identified.

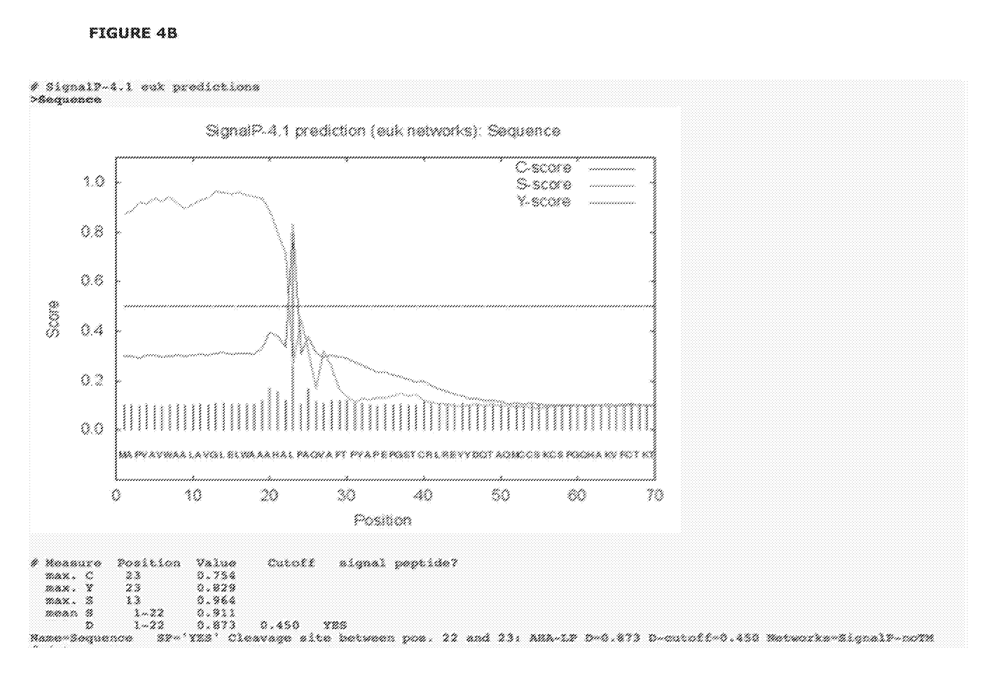

A signal peptide is predicted for translated EqDegR2 with cleavage in the analogous position to that predicted for the human p80 TNFR. The signal peptide cleavage site was predicted from SignalP software (http://www.cbs.dtu.dk/services/SignalP/). The SignalP algorithm employs a neural network for pattern recognition to predict a consensus sequence for the signal peptide. The signal peptide cleavage site is shown in FIG. 2B (see also FIG. 4B).

Cleavage of the signal sequence gives rise to a mature equine p80 TNFR polypeptide comprising an N-terminal “VPAQV” motif.
It is to be noted that the earlier database entry for equine p80 TNFR (TNFRSF1B; found here: http://www.ncbi.nlm.nih.gov/nuccore/XM_005607617) comprises the following amino acid sequence (SEQ ID NO:19):

SEQ ID NO: 19:

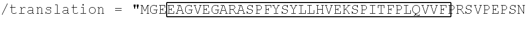

LCQPREYYDERAQRRCSQCPPGCRAKSFCNETSDTVCVPCEDSTYTQLWNWLPECLSC
GSRCSTGQVETQACTLKQNRICTCEPGRYCILPRQEGCQVCGLLRKCPPGFGVAKPGT
ATSNVVCAACAPGTFSDRTSSTDTCRPHRNCSSVAVPGNASMDAVCKSVLPTPRVASE
PASAPQPGSTRSHHAELTRGPSTAPGTSPLPPMVPSPPAEGLITGNFSLPIGLIVGVT
ALGLLVIGLVNCVIMTQKKKKSFCLQGEAKVPHLPADKAGGAPGQEQQHLLTTAQSSS
SSSLESSASTADKRAPTGDQLHAPGTGKASGPGEVRASSSSSAEPSSGSHGTQVNVTC
IVNVCSNSGHGSQCPSQTSSTAGDTDASPSDSLKDEQVPFSKEECPCQSQSGASETLL
QNPEEKPLPLGVPEGGVKSLTRPARDASQPRGADLWAF”

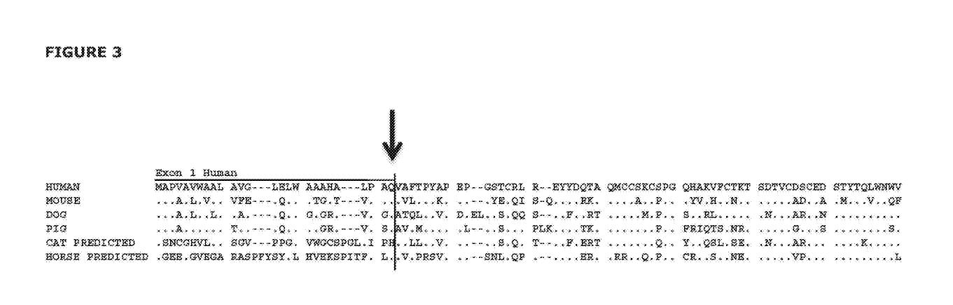

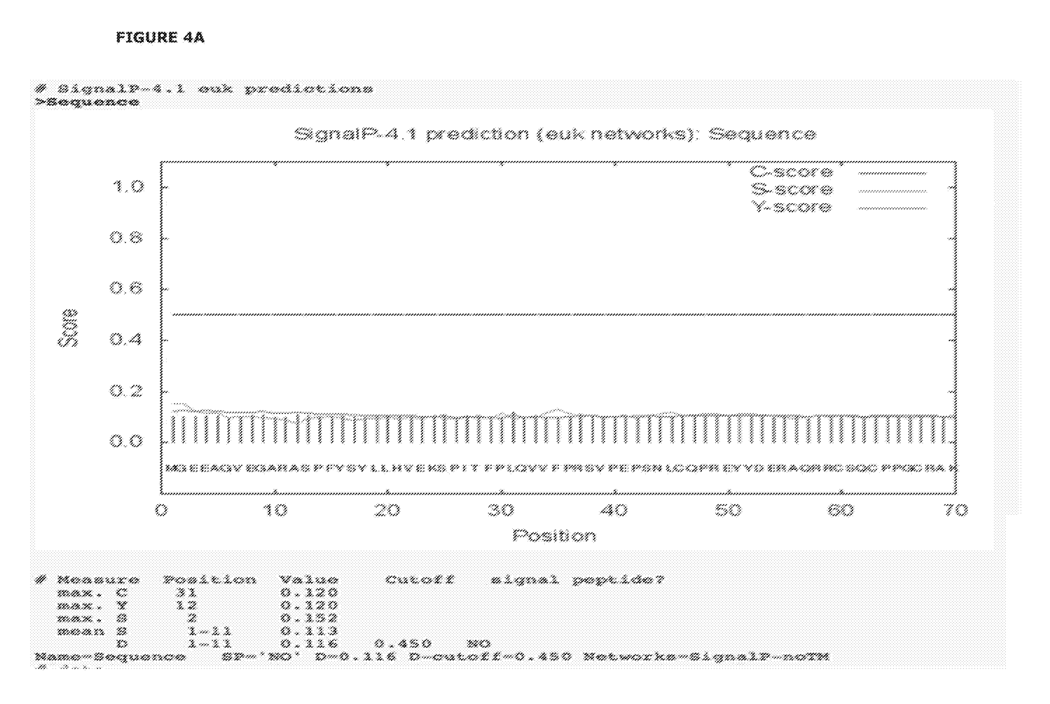

The boxed sequence is derived from exon 1 of the genome sequence (see FIG. 3) which, in the human, is located 21,626 bp upstream of exon 2. The inventors have found that, because of the large distance between exons 1 and 2 in the TNFR1B gene, exon prediction software will fail to identify the correct exon 1 in the genomic sequence of companion animals. This is evidenced in FIG. 4A. When the boxed sequence is entered into a signal peptide prediction program such as SignalP (http://www.cbs.dtu.dk/services/SignalP/), no signal peptide is predicted (see FIG. 4A). This is in contrast to the same analysis performed with the human p80 sequence (see FIGS. 2B and 4B).

The incorrect sequence shown the current database for equine p80 TNFR, with ‘leader’ sequence: MGEEAGVEGARASPFYSYLLHVEKSPITFPLQ, does not conform to the consensus and is not recognized as a signal peptide. A molecule lacking a signal peptide, as is the case for the predicted equine p80 TNFR sequence (XM_005607617), would not be secreted from the cell and would thus be non-functional.

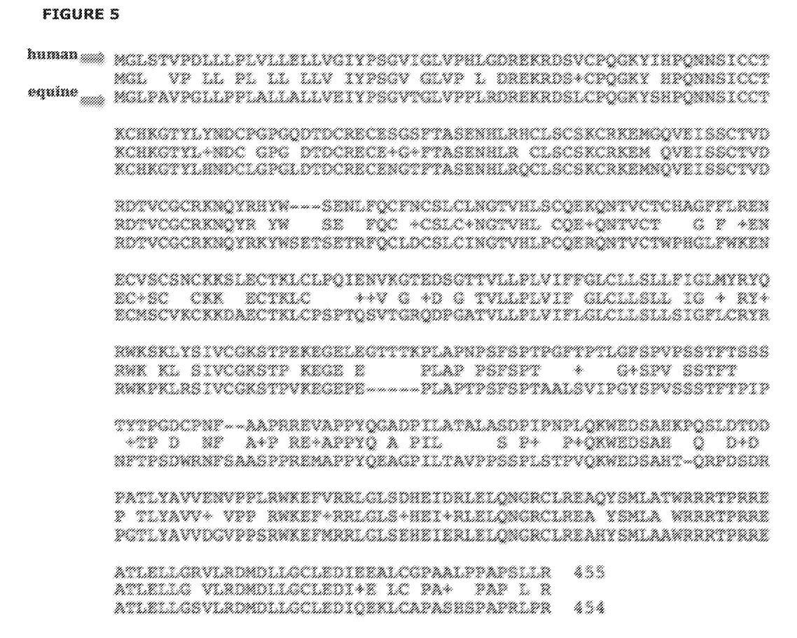

In contrast to the p80 isoform of equine TNFR, the p60 isoform (TNFR1A) is correctly predicted from the equine genomic sequence. The alignment between human and equine p60 sequences is shown in FIG. 5. It should be noted that the distance between exon 1 and exon 2 in the TNFR1A gene is only 7512 bp, as compared to a distance of 21,626 bp between exon 1 and exon 2 of the equine p80 TNFR1B gene.

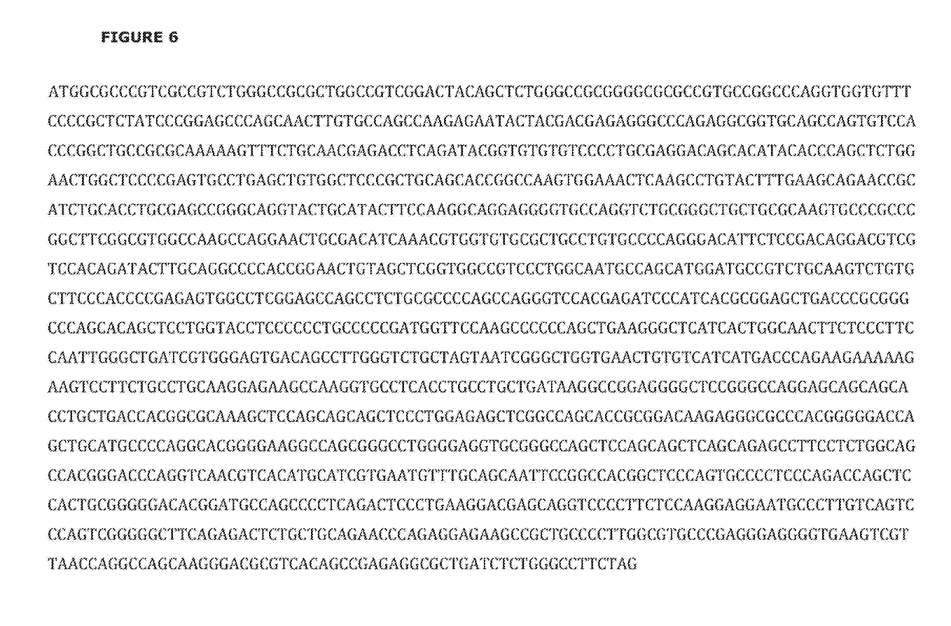

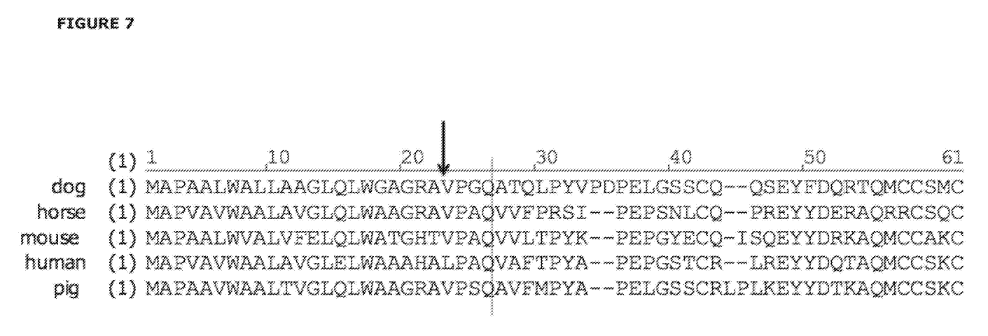

FIG. 6 shows the DNA sequence, EqDegR2, encoding exon 1 of equine p80 TNFR.

In summary, the amino acid sequence of the currently predicted equine TNFR p80 isoform (XM_005607617) was identified as an incomplete version that codes for an incomplete equine TNFR p80 extracellular domain that is incapable of being expressed in a mammalian cell, by virtue of it lacking a signal sequence and missing an N-terminal sequence of the mature peptide, VPAQV. By using a degenerate primer, EqDeg, designed from knowledge of the DNA sequences that encode the human, mouse, pig and canine N-terminal sequences of the p80 isoforms of TNFR, the present inventors have identified the correct N-terminal sequence for the p80 isoform of equine p80 TNFR.

### Example 2—Purification of Equine TNFR Polypeptides and Equine TNFR-Fc Fusion Proteins

CHO cells are transfected with expression plasmids encoding any one or more of SEQ ID NO:5, SEQ ID NO:16 and SEQ ID NO:17, resulting in the expression of a precursor equine TNFR p80 isoform, which is subsequently cleaved to release the signal sequence, generating a functional mature equine TNFR p80 isoform that can be tested and compared to an equivalent human TNFR polypeptide or to an equivalent human TNFR fusion protein polypeptide, where the fusion peptide comprises, for example, a human TNFR p80 extracellular domain fused N-terminally to a polypeptide comprising a hinge region and the CH2 and CH3 domains of human IgG.

An enzyme-linked immunosorbent assay (ELISA) is then used to determine binding of the generated functional equine TNFR polypeptide or fusion protein to equine TNF using, for example, a secondary anti-equine IgG polyclonal antibody-HRP conjugate.

The recombinant equine fusion protein can be purified from the supernatants by post-affinity capture on tandem Protein A and Protein G Sepharose columns, depending on the sequence of the IgG heavy chain constant region-derived fusion partner that has been employed. The present inventors have previously shown that Protein A chromatography can assist in the purification of equine IgG2 subtypes (HC2) and that equine-derived immunoglobulin heavy chain domains of type IgG2 are purified more efficiently than those of type IgG6. Accordingly, purification of fusion proteins according to an embodiment of the present invention provides higher yields where the fusion partner is derived from an equine IgG2 subtype, as compared to an equine IgG6 subtype.

Products of Protein A and/or Protein G affinity purification are then analysed by non-reducing SDS-PAGE gel electrophoresis to confirm the recovery (i.e., yield) of equine TNFR fusion proteins.

### Example 3—Inhibition of Equine TNF Activity

To determine whether the TNFR polypeptides or fusion proteins of the present invention, or fragments thereof, can inhibit TNF activity, a cell-based assay is used. 293-HEK cells transfected with the NF-kB-EGFP reporter construct pTRH1 (Vince et al., Cell, 131, 682, 2007) are exposed to equine TNF-alpha (R&D systems, 1 ng/ml) in the presence or absence of the TNFR polypeptide and/or fusion protein and/or fragment thereof. These cells respond to equine TNF by fluorescence, such that inhibition of binding is evidenced by reduced fluorescence as compared to the level of fluorescence in the absence of the TNFR polypeptide, fusion protein or fragment thereof, as is appropriate to the control group.

### Example 4—TNF Receptor Fusion Proteins Lack Complement Activity

A fusion protein according to the present invention is incubated with plates pre-coated with equine TNF (4 μg/ml). For comparison, a caninised monoclonal antibody (MAb, canine isotype HCB) with specificity to TNFR is incubated with plates coated with canine TNF. The plates are then washed and incubated with normal or heat-killed human serum as a source of complement. Binding of complement C1q is detected using a C1q reactive polyclonal antibody-HRP conjugate.

### Example 5—Feline TNFR p80 Polypeptide

As noted elsewhere herein, the inventors have identified, for the first time, the nucleic sequence for the p80 isoform of feline TNFR. The degenerate primer EqDeq, as described elsewhere herein, was designed from knowledge of the DNA sequences that encode the human, mouse, pig and canine N-terminal sequences of the p80 isoforms of TNFR. FIG. 1 shows the alignment of canine, human, murine (mouse) and porcine (pig) p80 TNFR nucleotide sequences used for the design of the degenerate sense primer, EqDeg.

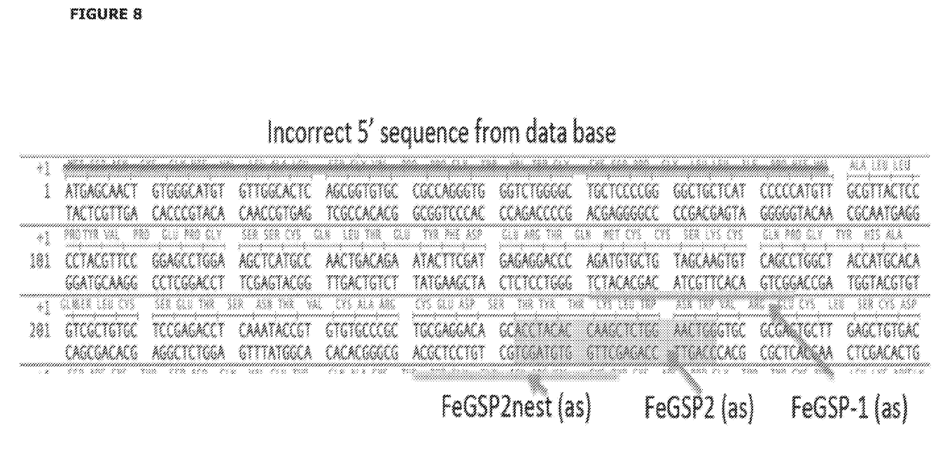

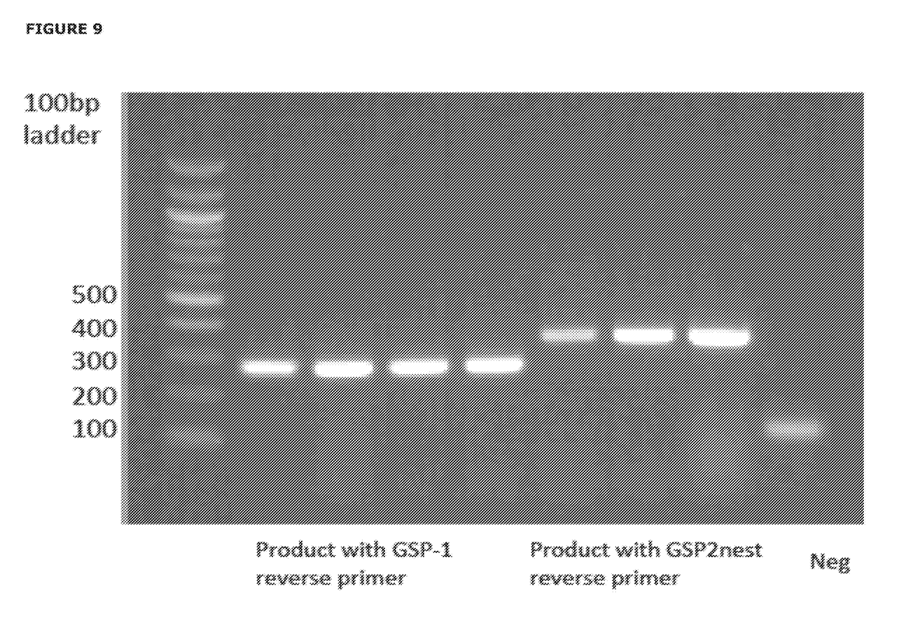

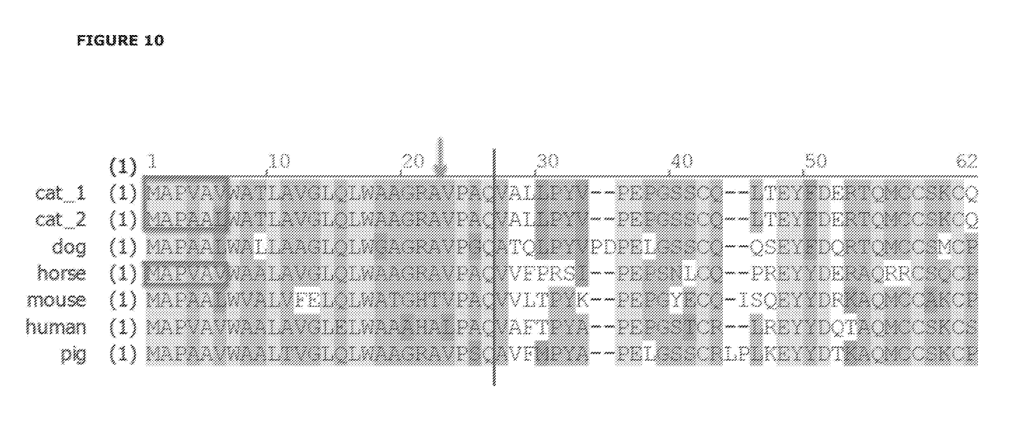

Total RNA was isolated from buffy coat cells prepared from 50 mls of feline blood. Specifically-primed first strand cDNA was prepared from 2 μg of the feline total RNA; the antisense primer FeGSP-1 (see FIG. 8) was used for priming first strand synthesis. This specifically-primed first strand cDNA was then used as a template for standard PCR amplification using the degenerate forward primer EqDeg, and a nested reverse primer FeGSP2nest. A PCR product of approximately 356 bp was obtained and this was subcloned and sequenced (see FIG. 9). An amino acid alignment of the translated sequenced PCR product is shown in FIG. 10.

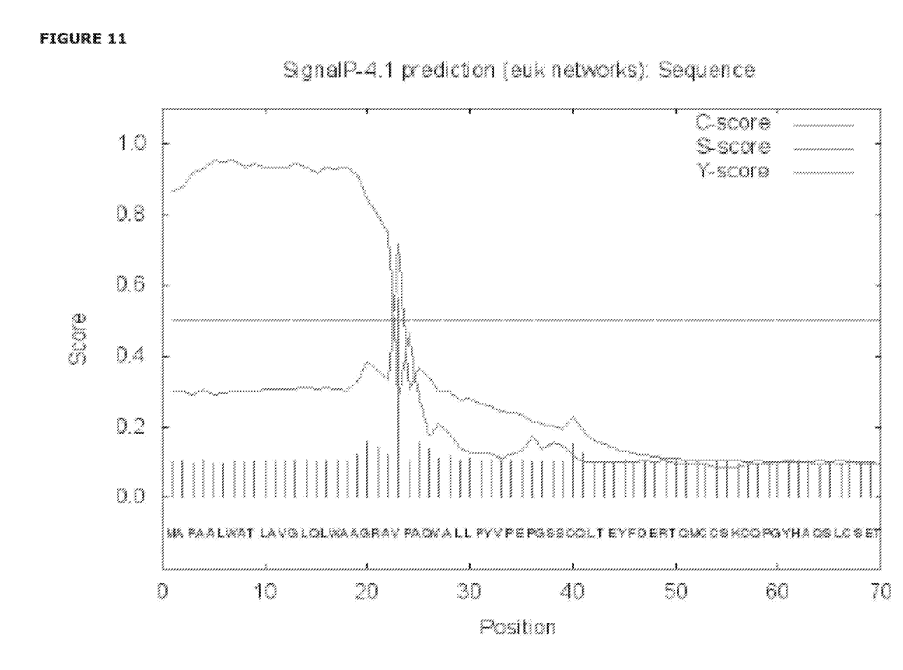

A signal peptide is predicted for the translated feline p80 isoform, as shown in FIG. 10 (see arrow). The signal peptide cleavage site was predicted from SignalP software (http://www.cbs.dtu.dk/services/SignalP/). The SignalP algorithm employs a neural network for pattern recognition to predict a consensus sequence for the signal peptide, as shown in FIG. 11.

Cleavage of the signal sequence gives rise to a mature feline p80 TNFR peptide comprising an N-terminal “VPAQV” motif.

### Example 6—Purification of Feline TNFR Fusion Proteins

CHO cells are transfected with expression plasmids encoding any one or more of SEQ ID NO:41, resulting in the expression of a fusion protein comprising a precursor feline TNFR p80 polypeptide, which is subsequently cleaved to release the signal sequence, generating a fusion protein comprising a functional mature feline TNFR p80 polypeptide that can be tested and compared to an equivalent human TNFR fusion protein polypeptide, where the fusion peptide comprises, for example, a human TNFR p80 extracellular domain fused N-terminally to a polypeptide comprising a hinge region and the CH2 and CH3 domains of human IgG.

An enzyme-linked immunosorbent assay (ELISA) is then used to determine the binding to feline TNF of the supernatants of expressed feline TNFR fusion proteins detected using a secondary anti-feline IgG polyclonal antibody-HRP conjugate.

Recombinant feline TNFR fusion proteins can then be purified from the supernatants by post-affinity capture on tandem Protein A and Protein G Sepharose columns, depending on the sequence of the Fc region of the chimeric fusion polypeptide. The present inventors have previously shown that Protein A chromatography can assist in the purification of feline IgG2 subtypes (HC2). Accordingly, purification of chimeric fusion polypeptides according to an embodiment of the present invention may be purified through a Protein A Sepharose column.

Products of Protein A affinity purification can then be analysed by non-reducing SD S-PAGE gel electrophoresis to confirm the recovery (i.e., yield) of the feline TNFR chimeric fusion proteins.

### Example 7—Inhibition of Feline TNF Activity

To determine whether the feline TNFR fusion proteins of the present invention, or fragments thereof, can inhibit TNF activity, a cell-based assay can be used. For example, 293-HEK cells transfected with the NF-kB-EGFP reporter construct pTRH1 (Vince et al., Cell, 131, 682, 2007) are exposed to feline TNF-alpha (R&D systems, 1 ng/ml) in the presence or absence of the feline TNFR fusion protein, and/or a fragment thereof. These cells respond to feline TNF by fluorescence, such that inhibition of binding is evidenced by reduced fluorescence as compared to the level of fluorescence in the absence of the TNFR fusion protein or fragment thereof, as is appropriate to the control group.

### Example 8—Feline TNF Receptor Fusion Proteins Lack Complement Activity

The feline fusion protein of the present invention is incubated with plates pre-coated with feline TNF (4 μg/ml). For comparison, a caninised monoclonal antibody (MAb, canine isotype HCB) with specificity to TNFR is incubated with plates coated with canine TNF. The plates are then washed and incubated with normal or heat-killed human serum as a source of complement. Binding of complement C1q is detected using a C1q reactive polyclonal antibody-HRP conjugate.

### Example 9—Purification of Feline TNFR Receptor Fusion Protein

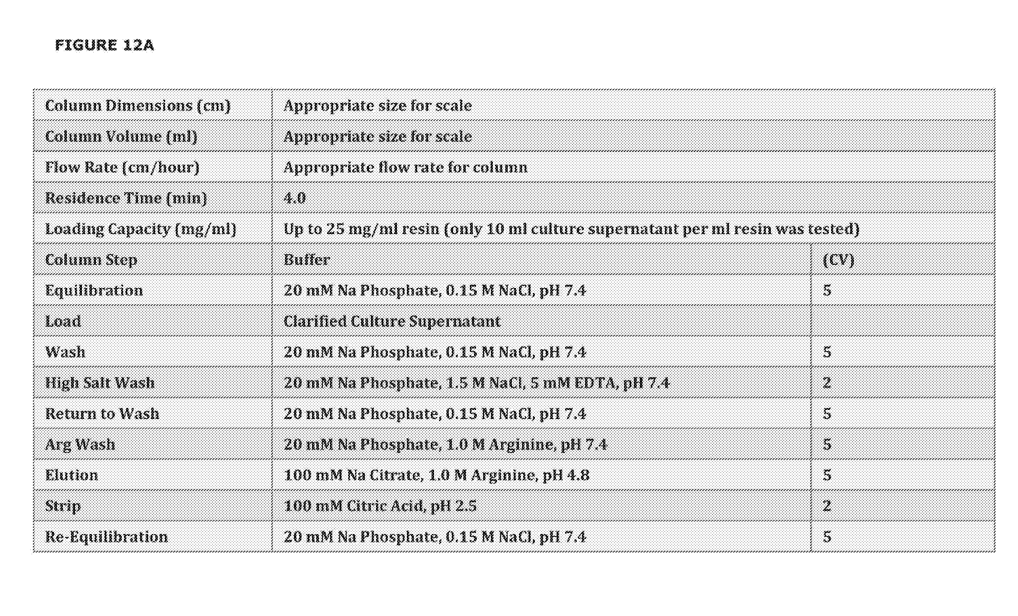

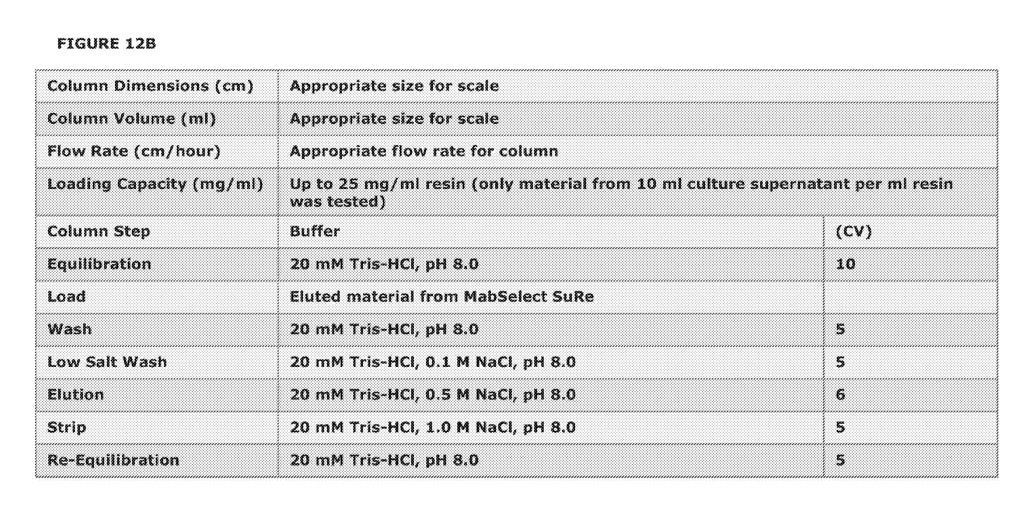

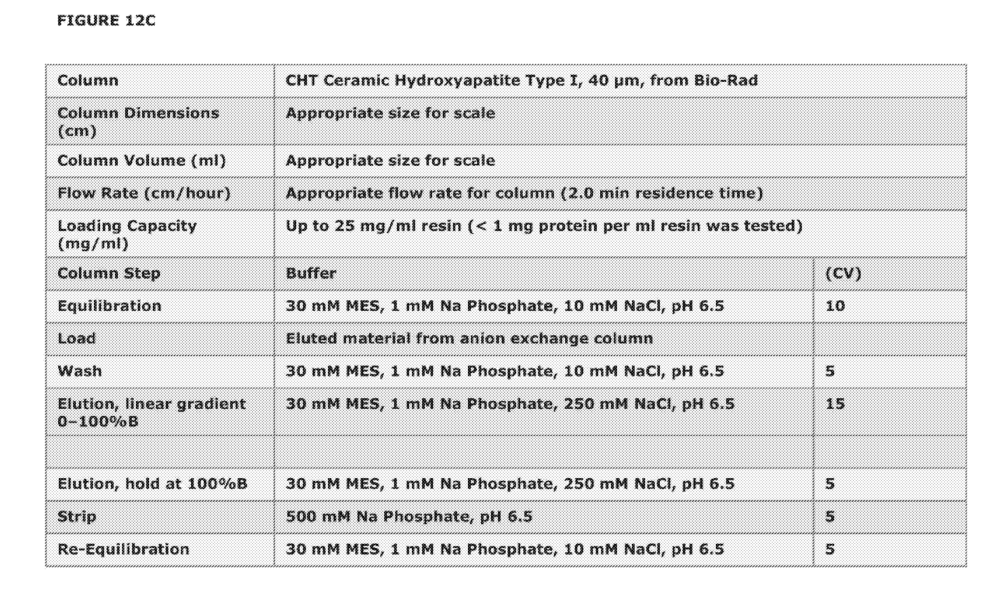

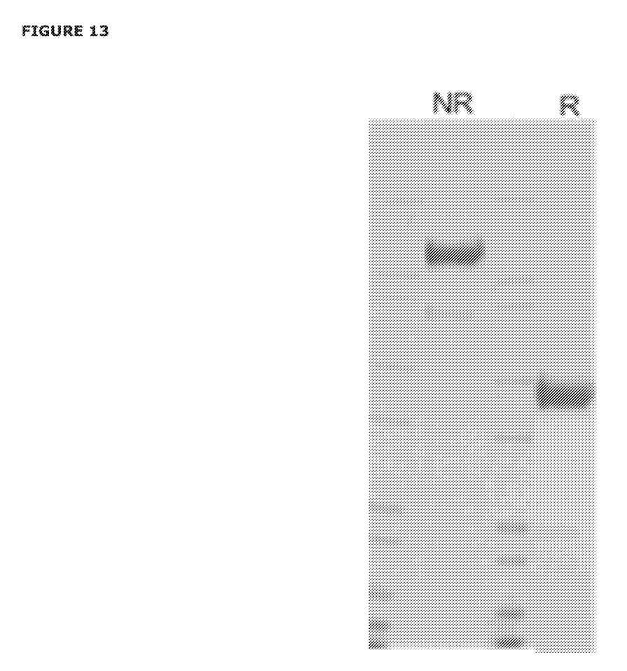

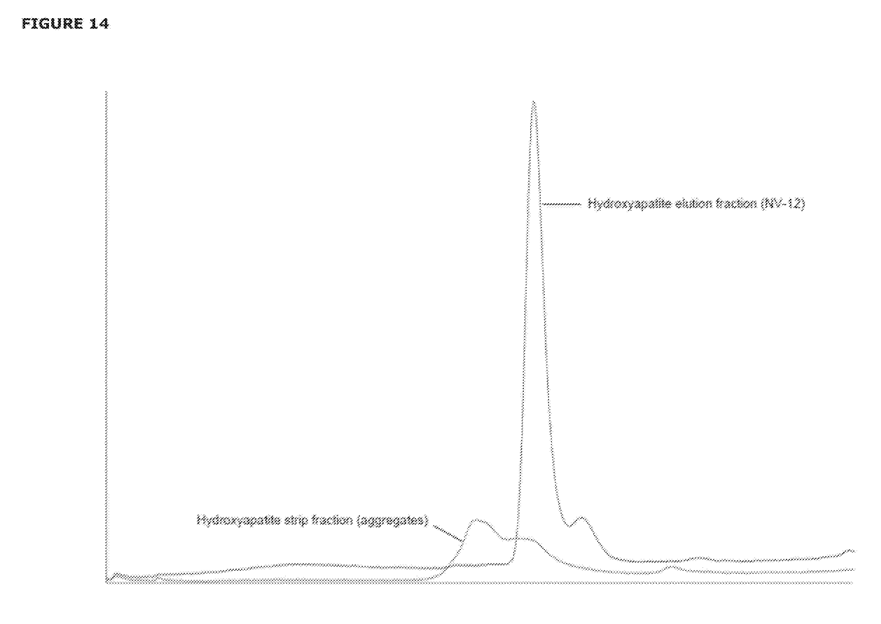

The feline TNFR p80-Fc fusion peptide, labelled NV-12 and having the amino acid sequence shown in SEQ ID NO:40, was purified by a 3-step process comprising (i) MabSelect SuRe chromatography (step 1; see FIG. 12A), HiTrap Q XL: anion exchange chromatography (step 2; see FIG. 12B) and CHT Ceramic Hydroxyapatite purification (step 3; FIG. 12C). The purified feline TNFR p80-Fc fusion peptide was then run on an SD S-PAGE gel under reducing and non-reducing conditions, as shown in FIG. 13. FIG. 14 shows the elution profile of the purified feline TNFR p80-Fc fusion peptide, NV-12, as obtained following the 3-step purification process, as outlined above and in FIGS. 12A-C.

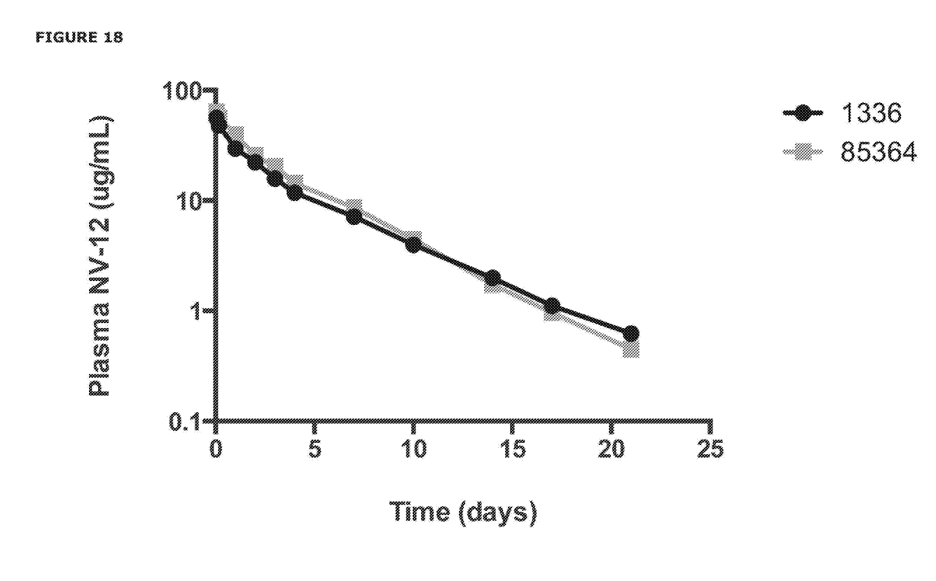

To determine the pharmacokinetics of the feline TNFR p80-Fc fusion peptide, NV-12, approximately 2 mg/kg body weight of NV-12 purified by the 3-step process disclosed herein was administered intravenously to two cats (subject nos. 1336 and 85364) and the plasma NV-12 level measured at various time points post-administration by enzyme-linked immunosorbent assay (ELISA). The data is presented in FIG. 18. The terminal half-life of NV-12 was estimated to be about 3.6 days.

### Example 10—Purification of Equine TNFR Receptor Fusion Protein

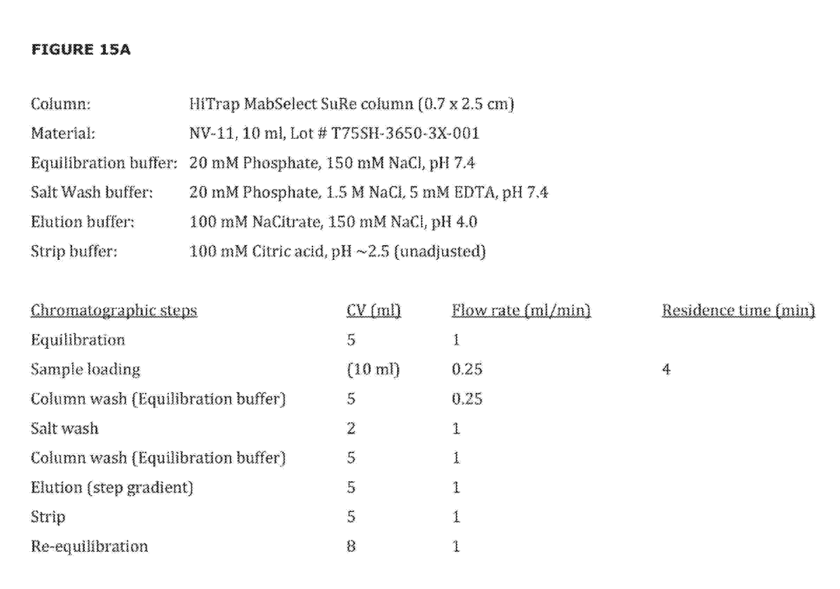

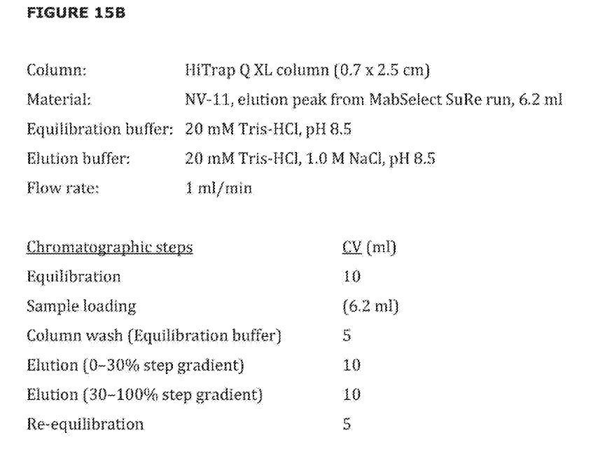

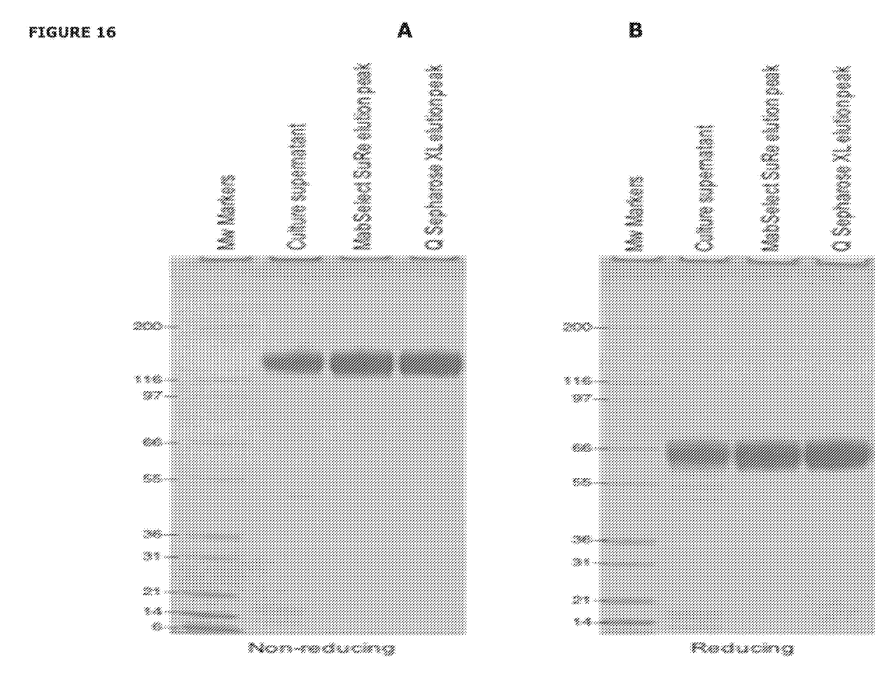

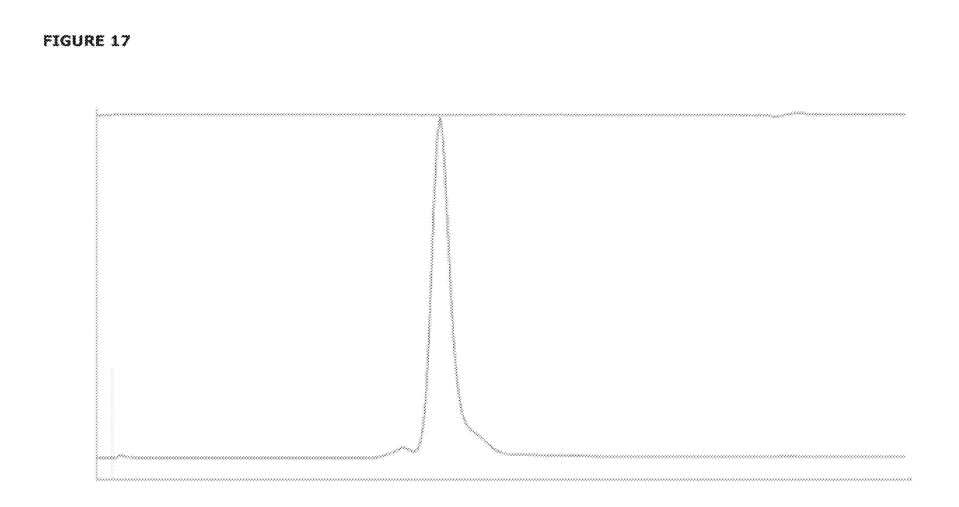

The equine TNFR p80-Fc fusion peptide, labelled NV-11 and having the amino acid sequence shown in SEQ ID NO:14, was purified by a 2-step process comprising MabSelect SuRe chromatography (step 1; see FIG. 15A) and Q Sepharose XL anion exchange chromatography (step 2; see FIG. 15B). The purified equine TNFR p80-Fc fusion peptide was then run on an SDS-PAGE gel under reducing and non-reducing conditions, as shown in FIG. 16. FIG. 17 shows the elution profile of the purified equine TNFR p80-Fc fusion peptide, NV-11, as obtained following the 2-step purification process, as outlined above and in FIGS. 15A and 15B.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### Brief Description of Sequences

SEQ ID NO:1 (truncated extracellular domain of equine TNFR p80 polypeptide comprising the CDR2 and CDR3 TNF-binding domains, as predicted from the CDR2 and CDR3 binding domains of the human TNFR p80 isoform, as described by Mukai et al. in Science Signaling 3 (148):ra83)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCA

SEQ ID NO:2 (extracellular domain of the mature equine TNFR p80 polypeptide)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGN

SEQ ID NO:3 (mature equine TNFR p80 isoform, including the extracellular domain (ECD; bolded and underlined) and the transmembrane domain)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNFSLPIGLIVGVTAL
GLLVIGLVNCVIMTQKKKKSFCLQGEAKVPHLPADKAGGAPGQEQQHLLT
TAQSSSSSSLESSASTADKRAPTGDQLHAPGTGKASGPGEVRASSSSSAE
PSSGSHGTQVNVTCIVNVCSNSGHGSQCPSQTSSTAGDTDASPSDSLKDE
QVPFSKEECPCQSQSGASETLLQNPEEKPLPLGVPEGGVKSLTRPARDAS
QPRGADLWAF

SEQ ID NO:4 (equine TNFR p80 signal sequence)

MAPVAVWAALAVGLQLWAAGRA

SEQ ID NO:5 (predicted precursor equine TNFR p80 polypeptide comprising a signal sequence (bolded+underlined), the ECD and the transmembrane domain)

MAPVAVWAALAVGLQLWAAGRAVPAQVVFPRSIPEPSNLCQPREYYDERA
QRRCSQCPPGCRAKSFCNETSDTVCVPCEDSTYTQLWNWLPECLSCGSRC
STGQVETQACTLKQNRICTCEPGRYCILPRQEGCQVCGLLRKCPPGFGVA
KPGTATSNVVCAACAPGTFSDRTSSTDTCRPHRNCSSVAVPGNASMDAVC
KSVLPTPRVASEPASAPQPGSTRSHHAELTRGPSTAPGTSPLPPMVPSPP
AEGLITGNFSLPIGLIVGVTALGLLVIGLVNCVIMTQKKKKSFCLQGEAK
VPHLPADKAGGAPGQEQQHLLTTAQSSSSSSLESSASTADKRAPTGDQLH
APGTGKASGPGEVRASSSSSAEPSSGSHGTQVNVTCIVNVCSNSGHGSQC
PSQTSSTAGDTDASPSDSLKDEQVPFSKEECPCQSQSGASETLLQNPEEK
PLPLGVPEGGVKSLTRPARDASQPRGADLWAF

SEQ ID NO:6 (nucleic acid coding (sense) sequence encoding equine TNFR p80 polypeptide)

ATGGCGCCCGTCGCCGTCTGGGCCGCGCTGGCCGTCGGACTACAGCTCTG
GGCCGCGGGGCGCGCCGTGCCGGCCCAGGTGGTGTTTCCCCGCTCTATCC
CGGAGCCCAGCAACTTGTGCCAGCCAAGAGAATACTACGACGAGAGGGCC
CAGAGGCGGTGCAGCCAGTGTCCACCCGGCTGCCGCGCAAAAAGTTTCTG
CAACGAGACCTCAGATACGGTGTGTGTCCCCTGCGAGGACAGCACATACA
CCCAGCTCTGGAACTGGCTCCCCGAGTGCCTGAGCTGTGGCTCCCGCTGC
AGCACCGGCCAAGTGGAAACTCAAGCCTGTACTTTGAAGCAGAACCGCAT
CTGCACCTGCGAGCCGGGCAGGTACTGCATACTTCCAAGGCAGGAGGGGT
GCCAGGTCTGCGGGCTGCTGCGCAAGTGCCCGCCCGGCTTCGGCGTGGCC
AAGCCAGGAACTGCGACATCAAACGTGGTGTGCGCTGCCTGTGCCCCAGG
GACATTCTCCGACAGGACGTCGTCCACAGATACTTGCAGGCCCCACCGGA
ACTGTAGCTCGGTGGCCGTCCCTGGCAATGCCAGCATGGATGCCGTCTGC
AAGTCTGTGCTTCCCACCCCGAGAGTGGCCTCGGAGCCAGCCTCTGCGCC
CCAGCCAGGGTCCACGAGATCCCATCACGCGGAGCTGACCCGCGGGCCCA
GCACAGCTCCTGGTACCTCCCCCCTGCCCCCGATGGTTCCAAGCCCCCCA
GCTGAAGGGCTCATCACTGGCAACTTCTCCCTTCCAATTGGGCTGATCGT
GGGAGTGACAGCCTTGGGTCTGCTAGTAATCGGGCTGGTGAACTGTGTCA
TCATGACCCAGAAGAAAAAGAAGTCCTTCTGCCTGCAAGGAGAAGCCAAG
GTGCCTCACCTGCCTGCTGATAAGGCCGGAGGGGCTCCGGGCCAGGAGCA
GCAGCACCTGCTGACCACGGCGCAAAGCTCCAGCAGCAGCTCCCTGGAGA
GCTCGGCCAGCACCGCGGACAAGAGGGCGCCCACGGGGGACCAGCTGCAT
GCCCCAGGCACGGGGAAGGCCAGCGGGCCTGGGGAGGTGCGGGCCAGCTC
CAGCAGCTCAGCAGAGCCTTCCTCTGGCAGCCACGGGACCCAGGTCAACG
TCACATGCATCGTGAATGTTTGCAGCAATTCCGGCCACGGCTCCCAGTGC
CCCTCCCAGACCAGCTCCACTGCGGGGGACACGGATGCCAGCCCCTCAGA
CTCCCTGAAGGACGAGCAGGTCCCCTTCTCCAAGGAGGAATGCCCTTGTC
AGTCCCAGTCGGGGGCTTCAGAGACTCTGCTGCAGAACCCAGAGGAGAAG
CCGCTGCCCCTTGGCGTGCCCGAGGGAGGGGTGAAGTCGTTAACCAGGCC
AGCAAGGGACGCGTCACAGCCGAGAGGCGCTGATCTCTGGGCCTTCTAG

SEQ ID NO: 7 (equine IgG1 heavy chain constant region; HC1; the hinge region and CH2 and CH3 domains are underlined)

ASTTAPKVFALAPGCGTTSDSTVALGCLVSGYFPEPVKVSWNSGSLTSGV
HTFPSVLQSSGFYSLSSMVTVPASSWTSETYICNVVHAASNFKVDKRIEP
IPDNHQKVCDMSKCPKCPAPELLGGPSVFIFPPNPKDTLMITRTPEVTCV
VVDVSQENPDVKFNWYMDGVEVRTATTRPKEEQFNSTYRVVSVLRIQHQD
WLSGKEFKCKVNNQALPQPIERTITKTKGRSQEPQVYVLAPHPDELSKSK
VSVTCLVKDFYPPEINIEWQSNGQPELETKYSTTQAQQDSDGSYFLYSKL
SVDRNRWQQGTTFTCGVMHEALHNHYTQKNVSKNPGK

SEQ ID NO: 8 (equine IgG2 heavy chain constant region; HC2; the hinge region and CH2 and CH3 domains are underlined)

ASTTAPKYFQLTPSCGITSDATVALGCLVSDYYPEPVTVSWNSGALTSGV
HTFPSVLQSSGLYALSSMVTVPASTWTSETYICNVAHPASSTKVDKRIPP
CVLSAEGVIPIPSVPKPQCPPYTHSKFLGGPSVFIFPPNPKDALMISRTP
VVTCVVVNLSDQYPDVQFSWYVDNTEVHSAITKQREAQFNSTYRVVSVLP
IQHQDWLSGKEFKCSVTNVGVPQPISRAISRGKGPSRVPQVYVLPPHPDE
LAKSKVSVTCLVKDFYPPDISVEWQSNRWPELEGKYSTTPAQLDGDGSYF
LYSKLSLETSRWQQVESFTCAVMHEALHNHFTKTDISESLGK

SEQ ID NO:9 (equine IgG3 heavy chain constant region; HC3; the hinge region and CH2 and CH3 domains are underlined)

STTAPKVFPLAPSCGNTSDSTVALGCLVSSYFPEPVTVSWNSGTLTSGVR
TFPSVLQSSGLYSLSSMVTVPASSLESKTYICNVAHPASSTKVDKRIEPV
LPKPTTPAPTVPLTTTVPVETTTPPCPCECPKCPAPELLGGPSVFIFPPK
PKDVLMITRMPEVTCLVVDVSHDSSDVLFTWYVDGTEVKTAKTMPNEEQN
NSTYRVVSVLRIQHQDWLNGKKFKCKVNNQALPAPVERTISKATGQTRVP
QVYVLAPHPDELSKNKVSVTCLVKDFYPTDITVEWQSNEHPEPEGKYRTT
EAQKDSDGSYFLYSKLTVEKDRWQQGTTFTCVVMHEALHNHVMQKNISKN
PGK

SEQ ID NO:10 (equine IgG4 heavy chain constant region; HC4; the hinge region and CH2 and CH3 domains are underlined)

ASTTAPKVFPLASHSAATSGSTVALGCLVSSYFPEPVTVSWNSGALTSGV
HTFPSVLQSSGLYSLSSMVTVPASSLKSQTYICNVAHPASSTKVDKKIVI
KECNGGCPAECLQVGPSVFIFPPKPKDVLMISRTPTVTCVVVDVGHDFPD
VQFNWYVDGVETHTATTEPKQEQFNSTYRVVSVLPIQHKDWLSGKEFKCK
VNNKALPAPVERTISKPTGQPREPQVYVLAPHRDELSKNKVSVTCLVKDF
YPTDIDIEWKSNGQPEPETKYSTTPAQLDSDGSYFLYSKLTVETNRWQQG
TTFTCAVMHEALHNHYTEKSVSKSPGK

SEQ ID NO:11 (equine IgG5 heavy chain constant region; HC5; the hinge region and CH2 and CH3 domains are underlined)

ESPKAPDVFPLTICGNTPDPTVPVGCLVSNYFPEPVTVSWNCDALKGDIH
TFPLDLSNSAHHSLSSMMAVPRSSLNQTYICSVAHPASSTKVDKRIVVKG
SPCPKCPAPELPGGPSVFIFPPKPKDVLKISRKPEVTCVVVDLGHDDPDV
QFTWFVDGVETHTATTEPKEEQFNSTYRVVSVLPIQHQDWLSGKEFKCSV
TNKALPAPVERTTSKAKGQLRVPQVYVLAPHPDELAKNTVSVTCLVKDFY
PPEIDVEWQSNEHPEPEGKYSTTPAQLNSDGSYFLYSKLSVETSRWKQGE
SFTCGVMHEAVENHYTQKNVSHSPGK

SEQ ID NO:12 (equine IgG6 heavy chain constant region; HC6; the hinge region and CH2 and CH3 domains are underlined)

ASTTAPKVFQLASHSAGTSDSTVALGCLVSSYFPEPVTVSWNSGALTSGV
HTFPSVRQSSGLYSLSSMVTVPASSLKSQTYICNVAHPASSTKVDKRIVI
KEPCCCPKCPGRPSVFIFPPNPKDTLMISRTPEVTCVVVDVSQENPDVKF
NWYVDGVEAHTATTKAKEKQDNSTYRWSVLPIQHQDWRRGKEFKCKVNNR
ALPAPVERTITKAKGELQDPKVYILAPHREEVTKNTVSVTCLVKDFYPPD
INVEWQSNEEPEPEVKYSTTPAQLDGDGSYFLYSKLTVETDRWEQGESFT
CWMHEAIRHTYROKSITNFPGK

SEQ ID NO:13 (equine IgG7 heavy chain constant region; HC7; the hinge region and CH2 and CH3 domains are underlined)

ASTTAPKVFPLASHSAATSGSTVALGCLVSSYFPEPVTVSWNSGALTSGV
HTFPSVLQSSGLYSLSSMVTVPASSLKSQTYICNVAHPASSTKVDKKIVI
KECGGCPTCPECLSVGPSVFIFPPKPKDVLMISRTPTVTCVVVDVGHDFP
DVQFNWYVDGVETHTATTEPKQEQNNSTYRVVSILAIQHKDWLSGKEFKC
KVNNQALPAPVQKTISKPTGQPREPQVYVLAPHRDELSKNKVSVTCLVKD
FYPTDIDIEWKSNGQPEPETKYSTTPAQLDSDGSYFLYSKLTVETNRWQQ
GTTFTCAVMHEALHNHYTEKSVSKSPGK

SEQ ID NO:14 (fusion protein, eqTNFRp80:HC2, comprising the ECD of mature equine TNFR p80 and equine IgG2 heavy chain constant region; the ECD of equine TNFR p80 is bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNASTTAPKYEQLTPS
CGITSDATVALGCLVSDYYPEPVTVSWNSGALTSGVHTFPSVLQSSGLYA
LSSMVTVPASTWTSETYICNVAHPASSTKVDKRIPPCVLSAEGVIPIPSV
PKPOCPPYTHSKFLGGPSVFIFPPNPKDALMISRTPVVTCVVVNLSDQYP
DVQFSWYVDNTEVHSAITKQREAQFNSTYRWSVLPIOHODWLSGKEFKCS
VINVGVPQPISRAISRGKGPSRVPOVYVLPPHPDELAKSKVSVTCLVKDF
YPPDISVEWQSNRWPELEGKYSTTPAOLDGOGSYFLYSKLSLETSRWOOV
ESFTCAVMHEALHNHFTKTDISESLGK

SEQ ID NO:15 (fusion protein, eqTNFRp80:HC6, comprising the ECD of mature equine TNFR p80 and equine IgG6 heavy chain constant region; the ECD of equine TNFR p80 is bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNASTTAPKVFQLASH
SAGTSDSTVALGCLVSSYFPEPVTVSWNSGALTSGVHTFPSVRQSSGLYS
LSSMVTVPASSLKSQTYICNVAHPASSTKVDKRIVIKEPCCCPKCPGRPS
VFIFPPNPKDTLMISRTPEVTCVVVDVSQENPDVKFNWYVDGVEAHTATT
KAKEKQDNSTYRWSVLPIQHQDWRRGKEFKCKVNNRALPAPVERTITKAK
GELQDPKVYILAPHREEVTKNTVSVTCLVKDFYPPDINVEWQSNEEPEPE
VKYSTTPAQLDGDGSYFLYSKLTVETDRWEQGESFTCWMHEAIRHTYROK
SITNFPGK

SEQ ID NO:16 (fusion protein, eqpreTNFRp80:HC2, comprising the ECD of the precursor equine TNFR p80 polypeptide and equine IgG2 heavy chain constant region; the signal peptide is bolded+underlined)

MAPVAVWAALAVGLQLWAAGRAVPAQVVFPRSIPEPSNLCQPREYYDERA
QRRCSQCPPGCRAKSFCNETSDTVCVPCEDSTYTQLWNWLPECLSCGSRC
STGQVETQACTLKQNRICTCEPGRYCILPRQEGCQVCGLLRKCPPGFGVA
KPGTATSNVVCAACAPGTFSDRTSSTDTCRPHRNCSSVAVPGNASMDAVC
KSVLPTPRVASEPASAPQPGSTRSHHAELTRGPSTAPGTSPLPPMVPSPP
AEGLITGNASTTAPKYEQLTPSCGITSDATVALGCLVSDYYPEPVTVSWN
SGALTSGVHTFPSVLQSSGLYALSSMVTVPASTWTSETYICNVAHPASST
KVDKRIPPCVLSAEGVIPIPSVPKPOCPPYTHSKFLGGPSVFIFPPNPKD
ALMISRTPVVTCVVVNLSDQYPDVQFSWYVDNTEVHSAITKQREAQFNST
YRWSVLPIOHODWLSGKEFKCSVINVGVPQPISRAISRGKGPSRVPOVYV
LPPHPDELAKSKVSVTCLVKDFYPPDISVEWQSNRWPELEGKYSTTPAOL
DGOGSYFLYSKLSLETSRWOOVESFTCAVM
HEALHNHFTKTDISESLGK

SEQ ID NO:17 (fusion protein, eqpreTNFRp80:HC6, comprising the ECD of the precursor equine TNFR p80 polypeptide and equine IgG6 heavy chain constant region; the signal peptide is bolded+underlined)

MAPVAVWAALAVGLQLWAAGRAVPAQVVFPRSIPEPSNLCQPREYYDERA
QRRCSQCPPGCRAKSFCNETSDTVCVPCEDSTYTQLWNWLPECLSCGSRC
STGQVETQACTLKQNRICTCEPGRYCILPRQEGCQVCGLLRKCPPGFGVA
KPGTATSNVVCAACAPGTFSDRTSSTDTCRPHRNCSSVAVPGNASMDAVC
KSVLPTPRVASEPASAPQPGSTRSHHAELTRGPSTAPGTSPLPPMVPSPP
AEGLITGNASTTAPKVFQLASHSAGTSDSTVALGCLVSSYFPEPVTVSWN
SGALTSGVHTFPSVRQSSGLYSLSSMVTVPASSLKSQTYICNVAHPASST
KVDKRIVIKEPCCCPKCPGRPSVFIFPPNPKDTLMISRTPEVTCVVVDVS
QENPDVKFNWYVDGVEAHTATTKAKEKQDNSTYRWSVLPIQHQDWRRGKE
FKCKVNNRALPAPVERTITKAKGELQDPKVYILAPHREEVTKNTVSVTCL
VKDFYPPDINVEWQSNEEPEPEVKYSTTPAQLDGDGSYFLYSKLTVETDR
WEQGESFTCWMHEAIRHTYROKSITNFPGK

SEQ ID NO:18 (leader sequence depicted in Genbank accession no. XM_005607617)

MGEEAGVEGARASPFYSYLLHVEKSPITFPLQ.

SEQ ID NO:19 (PREDICTED equine p80 TNFR, TNFRSF1B; XM_00507617)

/translation=“MGEEAGVEGARASPFYSYLLHVEKSPITFPLQVVFP
RSVPEPSNLCQPREYYDERAQRRCSQCPPGCRAKSFCNETSDTVCVPCED
STYTQLWNWLPECLSCGSRCSTGQVETQACTLKQNRICTCEPGRYCILPR
QEGCQVCGLLRKCPPGFGVAKPGTATSNVVCAACAPGTFSDRTSSTDTCR
PHRNCSSVAVPGNASMDAVCKSVLPTPRVASEPASAPQPGSTRSHHAELT
RGPSTAPGTSPLPPMVPSPPAEGLITGNFSLPIGLIVGVTALGLLVIGLV
NCVIMTQKKKKSFCLQGEAKVPHLPADKAGGAPGQEQQHLLTTAQSSSSS
SLESSASTADKRAPTGDQLHAPGTGKASGPGEVRASSSSSAEPSSGSHGT
QVNVTCIVNVCSNSGHGSQCPSQTSSTAGDTDASPSDSLKDEQVPFSKEE
CPCQSQSGASETLLQNPEEKPLPLGVPEGGVKSLTRPARDASQPRGADLW
AF”

SEQ ID NO:20 (fusion protein, eqpreTNFRp80:HC1, comprising the extracellular domain of the precursor equine TNFR p80 polypeptide, equine IgG1 hinge region and the CH2 and CH3 domains of equine IgG1 heavy chain constant region; the signal peptide is highlighted by the box)

| | |
|---|---|
| 1 | 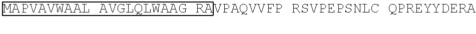 |
| 51 | QRRCSQCPPG CRAKSFCNET SDTVCVPCED STYTQLWNWL PECLSCGSRC |
| 101 | STGQVETQAC TLKQNRICTC EPGRYCILPR QEGCQVCGLL RKCPPGFGVA |
| 151 | KPGTATSNVV CAACAPGTFS DRTSSTDTCR PHRNCSSVAV PGNASMDAVC |
| 201 | KSVLPTPRVA SEPASAPQPG STRSHHAELT RGPSTAPGTS PLPPMVPSPP |
| 251 | AEGLITGNEP IPDNHQKVCD MSKCPKCPAP ELLGGPSVFI FPPNPKDTLM |
| 301 | ITRTPEVTCV VVDVSQENPD VKFNWYMDGV EVRTATTRPK EEQFNSTYRV |
| 351 | VSVLRIQHQD WLSGKEFKCK VNNQALPQPI ERTITKTKGR SQEPQVYVLA |
| 401 | PHPDELSKSK VSVTCLVKDF YPPEINIEWQ SNGQPELETK YSTTQAQQDS |
| 451 | DGSYFLYSKL SVDRNRWQQG TTFTCGVMHE ALHNHYTQKN VSKNPGK** |

SEQ ID NO:21 (fusion protein comprising the ECD of mature equine TNFR p80, equine IgG1 hinge region and the CH2 and CH3 domains of equine IgG1 heavy chain constant region; the ECD of mature equine TNFR p80 is bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNEPIPDNHQKVCDMS
KCPKCPAPELLGGPSVFIFPPNPKDTLMITRTPEVTCVVVDVSQENPDVK
FNWYMDGVEVRTATTRPKEEQFNSTYRVVSVLRIQHQDWLSGKEFKCKVN
NQALPQPIERTITKTKGRSQEPQVYVLAPHPDELSKSKVSVTCLVKDFYP
PEINIEWQSNGQPELETKYSTTQAQQDSDGSYFLYSKLSVDRNRWQQGTT
FTCGVMHEALHNHYTQKNVSKNPGK

SEQ ID NO:22 (fusion protein comprising the ECD of mature equine TNFR p80, equine IgG2 hinge region and the CH2 and CH3 domains of equine IgG2 heavy chain constant region; the ECD of mature equine TNFR p80 is bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNPPCVLSAEGVIPIP
SVPKPOCPPYTHSKFLGGPSVFIFPPNPKDALMISRTPVVTCVVVNLSDQ
YPDVQFSWYVDNTEVHSAITKQREAQFNSTYRWSVLPIOHODWLSGKEFK
CSVINVGVPQPISRAISRGKGPSRVPOVYVLPPHPDELAKSKVSVTCLVK
DFYPPDISVEWQSNRWPELEGKYSTTPAOLDGOGSYFLYSKLSLETSRWO
OVESFTCAVMHEALHNHFTKTDISESLGK

SEQ ID NO:23 (fusion protein comprising the ECD of mature equine TNFR p80, equine IgG3 hinge region and the CH2 and CH3 domains of equine IgG3 heavy chain constant region; the ECD of mature equine TNFR p80 is bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNEPVLPKPTTPAPTV
PLTTTVPVETTTPPCPCECPKCPAPELLGGPSVFIFPPKPKDVLMITRMP
EVTCLVVDVSHDSSDVLFTWYVDGTEVKTAKTMPNEEQNNSTYRVVSVLR
IQHQDWLNGKKFKCKVNNQALPAPVERTISKATGQTRVPQVYVLAPHPDE
LSKNKVSVTCLVKDFYPTDITVEWQSNEHPEPEGKYRTTEAQKDSDGSYF
LYSKLTVEKDRWQQGTTFTCVVMHEALHNHVMQKNISKNPGK

SEQ ID NO:24 (fusion protein comprising the ECD of mature equine TNFR p80, equine IgG4 hinge region and the CH2 and CH3 domains of equine IgG4 heavy chain constant region; the ECD of mature equine TNFR p80 is bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNVIKECNGGCPAECL
QVGPSVFIFPPKPKDVLMISRTPTVTCVVVDVGHDFPDVQFNWYVDGVET
HTATTEPKQEQFNSTYRVVSVLPIQHKDWLSGKEFKCKVNNKALPAPVER
TISKPTGQPREPQVYVLAPHRDELSKNKVSVTCLVKDFYPTDIDIEWKSN
GQPEPETKYSTTPAQLDSDGSYFLYSKLTVETNRWQQGTTFTCAVMHEAL
HNHYTEKSVSKSPGK

SEQ ID NO:25 (fusion protein comprising the ECD of mature equine TNFR p80, equine IgG5 hinge region and the CH2 and CH3 domains of equine IgG5 heavy chain constant region; the ECD of mature equine TNFR p80 is bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNVVKGSPCPKCPAPE
LPGGPSVFIFPPKPKDVLKISRKPEVTCVVVDLGHDDPDVQFTWFVDGVE
THTATTEPKEEQFNSTYRVVSVLPIQHQDWLSGKEFKCSVTNKALPAPVE
RTTSKAKGQLRVPQVYVLAPHPDELAKNTVSVTCLVKDFYPPEIDVEWQS
NEHPEPEGKYSTTPAQLNSDGSYFLYSKLSVETSRWKQGESFTCGVMHEA
VENHYTQKNVSHSPGK

SEQ ID NO:26 (fusion protein comprising the ECD of mature equine TNFR p80, equine IgG6 hinge region and the CH2 and CH3 domains of equine IgG6 heavy chain constant region; the ECD of mature equine TNFR p80 is bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNVIKEPCCCPKCPGR
PSVFIFPPNPKDTLMISRTPEVTCVVVDVSQENPDVKFNWYVDGVEAHTA
TTKAKEKQDNSTYRWSVLPIQHQDWRRGKEFKCKVNNRALPAPVERTITK
AKGELQDPKVYILAPHREEVTKNTVSVTCLVKDFYPPDINVEWQSNEEPE
PEVKYSTTPAQLDGDGSYFLYSKLTVETDRWEQGESFTCWMHEAIRHTYR
OKSITNFPGK

SEQ ID NO:27 (fusion protein comprising the ECD of mature equine TNFR p80, equine IgG7 hinge region and the CH2 and CH3 domains of equine IgG7 heavy chain constant region; the ECD of mature equine TNFR p80 is bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAACAPGTFSDR
TSSTDTCRPHRNCSSVAVPGNASMDAVCKSVLPTPRVASEPASAPQPGST
RSHHAELTRGPSTAPGTSPLPPMVPSPPAEGLITGNVIKECGGCPTCPEC
LSVGPSVFIFPPKPKDVLMISRTPTVTCVVVDVGHDFPDVQFNWYVDGVE
THTATTEPKQEQNNSTYRVVSILAIQHKDWLSGKEFKCKVNNQALPAPVQ
KTISKPTGQPREPQVYVLAPHRDELSKNKVSVTCLVKDFYPTDIDIEWKS
NGQPEPETKYSTTPAQLDSDGSYFLYSKLTVETNRWQQGTTFTCAVMHEA
LHNHYTEKSVSKSPGK

SEQ ID NO:28 (fusion protein comprising the predicted CDR2 and CDR3 TNF-binding domains of equine TNFR p80, equine IgG1 hinge region and the CH2 and CH3 domains of equine IgG1 heavy chain constant region; the predicted CDR2 and CDR3 TNF-binding domains of equine TNFR p80 are bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAEPIPDNHQKV
CDMSKCPKCPAPELLGGPSVFIFPPNPKDTLMITRTPEVTCVVVDVSQEN
PDVKFNWYMDGVEVRTATTRPKEEQFNSTYRVVSVLRIQHQDWLSGKEFK
CKVNNQALPQPIERTITKTKGRSQEPQVYVLAPHPDELSKSKVSVTCLVK
DFYPPEINIEWQSNGQPELETKYSTTQAQQDSDGSYFLYSKLSVDRNRWQ
QGTTFTCGVMHEALHNHYTQKNVSKNPGK

SEQ ID NO:29 (fusion protein comprising the predicted CDR2 and CDR3 TNF-binding domains of equine TNFR p80, equine IgG2 hinge region and the CH2 and CH3 domains of equine IgG2 heavy chain constant region; the predicted CDR2 and CDR3 TNF-binding domains of equine TNFR p80 are bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAPPCVLSAEGV
IPIPSVPKPOCPPYTHSKFLGGPSVFIFPPNPKDALMISRTPVVTCVVVN
LSDQYPDVQFSWYVDNTEVHSAITKQREAQFNSTYRWSVLPIOHODWLSG
KEFKCSVINVGVPQPISRAISRGKGPSRVPOVYVLPPHPDELAKSKVSVT
CLVKDFYPPDISVEWQSNRWPELEGKYSTTPAOLDGOGSYFLYSKLSLET
SRWOOVESFTCAVMHEALHNHFTKTDISESLGK

SEQ ID NO:30 (fusion protein comprising the predicted CDR2 and CDR3 TNF-binding domains of equine TNFR p80, equine IgG3 hinge region and the CH2 and CH3 domains of equine IgG3 heavy chain constant region; the predicted CDR2 and CDR3 TNF-binding domains of equine TNFR p80 are bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAEPVLPKPTTP
APTVPLTTTVPVETTTPPCPCECPKCPAPELLGGPSVFIFPPKPKDVLMI
TRMPEVTCLVVDVSHDSSDVLFTWYVDGTEVKTAKTMPNEEQNNSTYRVV
SVLRIQHQDWLNGKKFKCKVNNQALPAPVERTISKATGQTRVPQVYVLAP
HPDELSKNKVSVTCLVKDFYPTDITVEWQSNEHPEPEGKYRTTEAQKDSD
GSYFLYSKLTVEKDRWQQGTTFTCVVMHEALHNHVMQKNISKNPGK

SEQ ID NO:31 (fusion protein comprising the predicted CDR2 and CDR3 TNF-binding domains of equine TNFR p80, equine IgG4 hinge region and the CH2 and CH3 domains of equine IgG4 heavy chain constant region; the predicted CDR2 and CDR3 TNF-binding domains of equine TNFR p80 are bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAVIKECNGGCP
AECLQVGPSVFIFPPKPKDVLMISRTPTVTCVVVDVGHDFPDVQFNWYVD
GVETHTATTEPKQEQFNSTYRVVSVLPIQHKDWLSGKEFKCKVNNKALPA
PVERTISKPTGQPREPQVYVLAPHRDELSKNKVSVTCLVKDFYPTDIDIE
WKSNGQPEPETKYSTTPAQLDSDGSYFLYSKLTVETNRWQQGTTFTCAVM
HEALHNHYTEKSVSKSPGK

SEQ ID NO:32 (fusion protein comprising the predicted CDR2 and CDR3 TNF-binding domains of equine TNFR p80, equine IgG5 hinge region and the CH2 and CH3 domains of equine IgG5 heavy chain constant region; the predicted CDR2 and CDR3 TNF-binding domains of equine TNFR p80 are bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAVVKGSPCPKC
PAPELPGGPSVFIFPPKPKDVLKISRKPEVTCVVVDLGHDDPDVQFTWFV
DGVETHTATTEPKEEQFNSTYRVVSVLPIQHQDWLSGKEFKCSVTNKALP
APVERTTSKAKGQLRVPQVYVLAPHPDELAKNTVSVTCLVKDFYPPEIDV
EWQSNEHPEPEGKYSTTPAQLNSDGSYFLYSKLSVETSRWKQGESFTCGV
MHEAVENHYTQKNVSHSPGK

SEQ ID NO:33 (fusion protein comprising the predicted CDR2 and CDR3 TNF-binding domains of equine TNFR p80, equine IgG6 hinge region and the CH2 and CH3 domains of equine IgG6 heavy chain constant region; the predicted CDR2 and CDR3 TNF-binding domains of equine TNFR p80 are bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAVIKEPCCCPK
CPGRPSVFIFPPNPKDTLMISRTPEVTCVVVDVSQENPDVKFNWYVDGVE
AHTATTKAKEKQDNSTYRWSVLPIQHQDWRRGKEFKCKVNNRALPAPVER
TITKAKGELQDPKVYILAPHREEVTKNTVSVTCLVKDFYPPDINVEWQSN
EEPEPEVKYSTTPAQLDGDGSYFLYSKLTVETDRWEQGESFTCWMHEAIR
HTYROKSITNFPGK

SEQ ID NO:34 (fusion protein comprising the predicted CDR2 and CDR3 TNF-binding domains of equine TNFR p80, equine IgG7 hinge region and the CH2 and CH3 domains of equine IgG7 heavy chain constant region; the predicted CDR2 and CDR3 TNF-binding domains of equine TNFR p80 are bolded+underlined)

VPAQVVFPRSIPEPSNLCQPREYYDERAQRRCSQCPPGCRAKSFCNETSD
TVCVPCEDSTYTQLWNWLPECLSCGSRCSTGQVETQACTLKQNRICTCEP
GRYCILPRQEGCQVCGLLRKCPPGFGVAKPGTATSNVVCAVIKECGGCPT
CPECLSVGPSVFIFPPKPKDVLMISRTPTVTCVVVDVGHDFPDVQFNWYV
DGVETHTATTEPKQEQNNSTYRVVSILAIQHKDWLSGKEFKCKVNNQALP
APVQKTISKPTGQPREPQVYVLAPHRDELSKNKVSVTCLVKDFYPTDIDI
EWKSNGQPEPETKYSTTPAQLDSDGSYFLYSKLTVETNRWQQGTTFTCAV
MHEALHNHYTEKSVSKSPGK

SEQ ID NO:35 (equine IgG1 hinge region)

EPIPDNHQKVCDMSKCPKCP

SEQ ID NO:36 (truncated extracellular domain of feline TNFR p80 polypeptide comprising the CDR2 and CDR3 TNF-binding domains, as predicted from the CDR2 and CDR3 binding domains of the human TNFR p80 isoform, as described by Mukai et al. in Science Signaling 3 (148):ra83)

RCEDSTYTKLWNWVRECLSCDSRCTSDQVETQACTPEQNRVCTCRPGWYC
TLKRQKGCRLCAPLHRCRPGFGVTRPGTATSNVACA

SEQ ID NO:37 (extracellular domain of the mature feline TNFR p80 polypeptide, including the N-terminal VPAQV motif)

VPAQVALLPYVPEPGSSCQLTEYFDERTQMCCSKCQPGYHAQSLCSETSN
TVCARCEDSTYTKLWNWVRECLSCDSRCTSDQVETQACTPEQNRVCTCRP
GWYCTLKRQKGCRLCAPLHRCRPGFGVTRPGTATSNVACAPCGPGTFSDT
TSSTDVCRPHRICGSVAIPGNATMDAVCASVPPTLRMAPRPAPTSQPAST
LSQQVEPTPGPRTAPSTSLLFVMVPTPPAEGLSTGD

SEQ ID NO:38 (feline TNFR p80 signal sequence)

MAPVAVWATLAVGLQLWAAGRA

SEQ ID NO:39 (alternative feline TNFR p80 signal sequence)

MAPAALWATLAVGLQLWAAGRA

SEQ ID NO:40 (fusion protein, feTNFRp80:HC1, comprising the extracellular domain of the mature feline TNFR p80 polypeptide and the hinge region, CH2 and CH3 domains of the feline IgG1 heavy chain constant region (amino acid residues 237-473))

VPAQVALLPYVPEPGSSCQLTEYFDERTQMCCSKCQPGYHAQSLCSETSN
TVCARCEDSTYTKLWNWVRECLSCDSRCTSDQVETQACTPEQNRVCTCRP
GWYCTLKRQKGCRLCAPLHRCRPGFGVTRPGTATSNVACAPCGPGTFSDT
TSSTDVCRPHRICGSVAIPGNATMDAVCASVPPTLRMAPRPAPTSQPAST
LSQQVEPTPGPRTAPSTSLLFVMVPTPPAEGLSTGDRKTDHPPGPKPCDC
PKCPPPEMLGGPSIFIFPPKPKDTLSISRTPEVTCLVVDLGPDDSDVQIT
WFVDNTQVYTAKTSPREEQFNSTYRVVSVLPILHQDWLKGKEFKCKVNSK
SLPSPIERTISKAKGQPHEPQVYVLPPAQEELSRNKVSVTCLIKSFHPPD
IAVEWEITGQPEPENNYRTTPPQLDSDGTYFVYSKLSVDRSHWQRGNTYT
CSVSHEALHSHHTQKSLTQSPGK**

SEQ ID NO:41 (fusion protein, fepreTNFRp80:HC1, comprising the extracellular domain of the precursor feline TNFR p80 polypeptide, feline IgG1 hinge region and the CH2 and CH3 domains of the feline IgG1 heavy chain constant region; the signal peptide (SEQ ID NO:38) is underlined)

MAPVAVWATLAVGLQLWAAGRAVPAQVALLPYVPEPGSSCQLTEYFDERT
QMCCSKCQPGYHAQSLCSETSNTVCARCEDSTYTKLWNWVRECLSCDSRC
TSDQVETQACTPEQNRVCTCRPGWYCTLKRQKGCRLCAPLHRCRPGFGVT
RPGTATSNVACAPCGPGTFSDTTSSTDVCRPHRICGSVAIPGNATMDAVC
ASVPPTLRMAPRPAPTSQPASTLSQQVEPTPGPRTAPSTSLLFVMVPTPP
AEGLSTGDRKTDHPPGPKPCDCPKCPPPEMLGGPSIFIFPPKPKDTLSIS
RTPEVTCLVVDLGPDDSDVQITWFVDNTQVYTAKTSPREEQFNSTYRVVS
VLPILHQDWLKGKEFKCKVNSKSLPSPIERTISKAKGQPHEPQVYVLPPA
QEELSRNKVSVTCLIKSFHPPDIAVEWEITGQPEPENNYRTTPPQLDSDG
TYFVYSKLSVDRSHWQRGNTYTCSVSHEALHSHHTQKSLTQSPGK**

SEQ ID NO:42 (nucleic acid coding (sense) sequence encoding feline TNFR p80 polypeptide)

ATGGCGCCCGTCGCCGTGTGGGCCACGCTGGCCGTCGGACTGCAGCTCTG
GGCCGCGGGGCGCGCCGTGCCCGCCCAGGTTGCGTTACTCCCCTACGTTC
CGGAGCCTGGAAGCTCATGCCAACTGACAGAATACTTCGATGAGAGGACC
CAGATGTGCTGTAGCAAGTGTCAGCCTGGCTACCATGCACAGTCGCTGTG
CTCCGAGACCTCAAATACCGTGTGTGCCCGCTGCGAGGACAGCACCTACA
CCAAGCTCTGGAACTGGGTGCGCGAGTGCTTGAGCTGTGACTCCCGCTGC
ACCTCTGACCAAGTGGAGACTCAGGCCTGCACTCCGGAACAGAACCGCGT
CTGCACCTGCAGGCCGGGCTGGTACTGCACGTTGAAGAGGCAGAAAGGGT
GCCGGCTGTGTGCGCCCCTGCACAGGTGCCGCCCGGGCTTCGGCGTGACC
AGGCCAGGAACTGCAACGTCAAATGTGGCGTGCGCTCCCTGTGGCCCAGG
GACGTTCTCCGACACAACGTCGTCCACAGATGTCTGCAGGCCCCACCGGA
TCTGTGGCTCAGTGGCCATCCCTGGCAATGCAACCATGGATGCCGTCTGC
GCATCTGTGCCTCCTACCCTGAGGATGGCCCCACGCCCAGCCCCCACGTC
CCAGCCAGCGTCTACACTATCCCAGCAAGTGGAGCCGACTCCAGGCCCTC
GCACGGCCCCTAGCACCTCCCTTCTGTTTGTGATGGTCCCAACCCCCCCA
GCTGAAGGGCTCAGCACGGGCGACATCTCTCTCCTCATTGGACTGATTGT
GGCTGTGACATCCTTGGGTCTGGTGATCATAGGGCTGGTGAAATGTGTCA
TTATGACCCAGAAAAAAAAGAAGCCCTTCTGTCTACAAGGAGAAGCCAAA
GTGCCTCACCTGCCTGCTGACAAGGCCCGAAGTGCCCCCGGCCCCGAGCA
GCAGCACCTGCTGACCACAGCGCCCAGCTCCAGCAGCAGCTCCCTGGAGA
GCTCAGGCAGCACCGCAGACGGGAGGGCGCCCACCGGGACCCGGCTGCCC
GCACCAGGCACGGAGAAGGCCGCCGGGTCTGGGGAGGCCTGGGCCAGCTC
CAGCAGCTCAGAGCCTTCCTCTGGCAGCCACGGGACCCAGGTCAACGTCA
CATGCATCGTGAATGTGTGCAGCGCCTCCGACCACGGCTCTCAGTGCGCC
TCCCAGGCCAGCTACACGATGGGGGACGTGGATGCCAGCTCCTCCGGCTC
CCCAAATGACGAGCAGGTCCCCTTCTCCAAGGAAGAATGCCCTTTTCAGT
CTCAGCCAGGGGCCCCGGAGACTCTGCTGGAGAACCCGGAGGAGAAGCCC
CTGCCCCTTGGTGTGCCTGATGCTGGGATGAAGCCCAGTTAG

SEQ ID NO:43 (feline IgG1a heavy chain constant region; GenBank Accession No. AB0167710; the hinge region, CH2 and CH3 domains are underlined)

ASTTAPSVFPLAPSCGTTSGATVALACLVLGYFPEPVTVSWNSGALTSGV
HTFPAVLQASGLYSLSSMVTVPSSRWLSDTFTCNVAHPPSNTKVDKTVRK
TDHPPGPKPCDCPKCPPPEMLGGPSIFIFPPKPKDTLSISRTPEVTCLVV
DLGPDDSDVQITWFVDNTQVYTAKTSPREEQFNSTYRVVSVLPILHQDWL
KGKEFKCKVNSKSLPSPIERTISKAKGQPHEPQVYVLPPAQEELSRNKVS
VTCLIKSFHPPDIAVEWEITGQPEPENNYRTTPPQLDSDGTYFVYSKLSV
DRSHWQRGNTYTCSVSHEALHSHHTQKSLTQSPGK

SEQ ID NO:44 (feline IgG1b heavy chain constant region; GenBank Accession No. AB016711; the hinge region, CH2 and CH3 domains are underlined)

ASTTAPSVFPLAPSCGTTSGATVALACLVLGYFPEPVTVSWNSGALTSGV
HTFPAVLQASGLYSLSSMVTVPSSRWLSDTFTCNVAHPPSNTKVDKTVRK
TDHPPGPKPCDCPKCPPPEMLGGPSIFIFPPKPKDTLSISRTPEVTCLVV
DLGPDDSDVQITWFVDNTQVYTAKTSPREEQFNSTYRVVSVLPILHQDWL
KGKEFKCKVNSKSLPSPIERTISKDKGQPHEPQVYVLPPAQEELSRNKVS
VTCLIEGFYPSDIAVEWEITGQPEPENNYRTTPPQLDSDGTYFLYSRLSV
DRSRWQRGNTYTCSVSHEALHSHHTQKSLTQSPGK

SEQ ID NO:45 (feline IgG2 heavy chain constant region; GenBank Accession No. KF811175; the hinge region, CH2 and CH3 domains are underlined)

ASTTASSVFPLAPSCGTTSGATVALACLVLGYFPEPVTVSWNSGALTSGV
HTFPSVLQASGLYSLSSMVTVPSSRWLSDTFTCNVAHRPSSTKVDKTVPK
TASTIESKTGEGPKCPVPEIPGAPSVFIFPPKPKDTLSISRTPEVTCLVV
DLGPDDSNVQITWFVDNTEMHTAKTRPREEQFNSTYRVVSVLPILHQDWL
KGKEFKCKVNSKSLPSAMERTISKAKGQPHEPQVYVLPPTQEELSENKVS
VTCLIKGFHPPDIAVEWEITGQPEPENNYQTTPPQLDSDGTYFLYSRLSV
DRSHWQRGNTYTCSVSHEALHSHHTQKSLTQSPGK

SEQ ID NO:46 (fusion protein comprising feline TNFR p80 (bolded), the hinge region, CH2 and CH3 domains of the feline IgG1a heavy chain constant region (underlined))

VPAQVALLPYVPEPGSSCQLTEYFDERTQMCCSKCQPGYHAQSLCSETSN
TVCARCEDSTYTKLWNWVRECLSCDSRCTSDQVETQACTPEQNRVCTCRP
GWYCTLKRQKGCRLCAPLHRCRPGFGVTRPGTATSNVACAPCGPGTFSDT
TSSTDVCRPHRICGSVAIPGNATMDAVCASVPPTLRMAPRPAPTSQPAST
LSQQVEPTPGPRTAPSTSLLFVMVPTPPAEGLSTGDRKTDHPPGPKPCDC
PKCPPPEMLGGPSIFIFPPKPKDTLSISRTPEVTCLVVDLGPDDSDVQIT
WFVDNTQVYTAKTSPREEQFNSTYRVVSVLPILHQDWLKGKEFKCKVNSK
SLPSPIERTISKAKGQPHEPQVYVLPPAQEELSRNKVSVTCLIKSFHPPD
IAVEWEITGQPEPENNYRTTPPQLDSDGTYFVYSKLSVDRSHWQRGNTYT
CSVSHEALHSHHTQKSLTQSPGK

SEQ ID NO:47 (fusion protein comprising feline TNFR p80 (bolded) and the hinge region, CH2 and CH3 domains of the feline IgG1b heavy chain constant region (underlined))

VPAQVALLPYVPEPGSSCQLTEYFDERTQMCCSKCQPGYHAQSLCSETSN
TVCARCEDSTYTKLWNWVRECLSCDSRCTSDQVETQACTPEQNRVCTCRP
GWYCTLKRQKGCRLCAPLHRCRPGFGVTRPGTATSNVACAPCGPGTFSDT
TSSTDVCRPHRICGSVAIPGNATMDAVCASVPPTLRMAPRPAPTSQPAST
LSQQVEPTPGPRTAPSTSLLFVMVPTPPAEGLSTGDRKTDHPPGPKPCDC
PKCPPPEMLGGPSIFIFPPKPKDTLSISRTPEVTCLVVDLGPDDSDVQIT
WFVDNTQVYTAKTSPREEQFNSTYRVVSVLPILHQDWLKGKEFKCKVNSK
SLPSPIERTISKDKGQPHEPQVYVLPPAQEELSRNKVSVTCLIEGFYPSD
IAVEWEITGQPEPENNYRTTPPQLDSDGTYFLYSRLSVDRSRWQRGNTYT
CSVSHEALHSHHTQKSLTQSPGK

SEQ ID NO:48 (fusion protein comprising feline TNFR p80 (bolded) and the hinge region, CH2 and CH3 domains of the feline IgG2 heavy chain constant region (underlined))

VPAQVALLPYVPEPGSSCQLTEYFDERTQMCCSKCQPGYHAQSLCSETSN
TVCARCEDSTYTKLWNWVRECLSCDSRCTSDQVETQACTPEQNRVCTCRP
GWYCTLKRQKGCRLCAPLHRCRPGFGVTRPGTATSNVACAPCGPGTFSDT
TSSTDVCRPHRICGSVAIPGNATMDAVCASVPPTLRMAPRPAPTSQPAST
LSQQVEPTPGPRTAPSTSLLFVMVPTPPAEGLSTGDPKTASTIESKTGEG
PKCPVPEIPGAPSVFIFPPKPKDTLSISRTPEVTCLVVDLGPDDSNVQIT
WFVDNTEMHTAKTRPREEQFNSTYRVVSVLPILHQDWLKGKEFKCKVNSK
SLPSAMERTISKAKGQPHEPQVYVLPPTQEELSENKVSVTCLIKGFHPPD
IAVEWEITGQPEPENNYQTTPPQLDSDGTYFLYSRLSVDRSHWQRGNTYT
CSVSHEALHSHHTQKSLTQSPGK

SEQ ID NO:49 (fusion protein comprising a truncated feline TNFR p80 TNF-binding region (bolded), the hinge region, CH2 and CH3 domains of the feline IgG1a heavy chain constant region (underlined))

VPAQVALLPYVPEPGSSCQLTEYFDERTQMCCSKCQPGYHAQSLCSETSN
TVCARCEDSTYTKLWNWVRECLSCDSRCTSDQVETQACTPEQNRVCTCRP
GWYCTLKRQKGCRLCAPLHRCRPGFGVTRPGTATSNVACARKTDHPPGPK
PCDCPKCPPPEMLGGPSIFIFPPKPKDTLSISRTPEVTCLVVDLGPDDSD
VQITWFVDNTQVYTAKTSPREEQFNSTYRVVSVLPILHQDWLKGKEFKCK
VNSKSLPSPIERTISKAKGQPHEPQVYVLPPAQEELSRNKVSVTCLIKSF
HPPDIAVEWEITGQPEPENNYRTTPPQLDSDGTYFVYSKLSVDRSHWQRG
NTYTCSVSHEALHSHHTQKSLTQSPGK

SEQ ID NO:50 (fusion protein comprising a truncated feline TNFR p80 TNF-binding region (bolded) and the hinge region, CH2 and CH3 domains of the feline IgG1b heavy chain constant region (underlined))

VPAQVALLPYVPEPGSSCQLTEYFDERTQMCCSKCQPGYHAQSLCSETSN
TVCARCEDSTYTKLWNWVRECLSCDSRCTSDQVETQACTPEQNRVCTCRP
GWYCTLKRQKGCRLCAPLHRCRPGFGVTRPGTATSNVACARKTDHPPGPK
PCDCPKCPPPEMLGGPSIFIFPPKPKDTLSISRTPEVTCLVVDLGPDDSD
VQITWFVDNTQVYTAKTSPREEQFNSTYRVVSVLPILHQDWLKGKEFKCK
VNSKSLPSPIERTISKDKGQPHEPQVYVLPPAQEELSRNKVSVTCLIEGF
YPSDIAVEWEITGQPEPENNYRTTPPQLDSDGTYFLYSRLSVDRSRWQRG
NTYTCSVSHEALHSHHTQKSLTQSPGK

SEQ ID NO:51 (fusion protein comprising a truncated feline TNFR p80 TNF-binding region (bolded) and the hinge region, CH2 and CH3 domains of the feline IgG2 heavy chain constant region (underlined))

VPAQVALLPYVPEPGSSCQLTEYFDERTQMCCSKCQPGYHAQSLCSETSN
TVCARCEDSTYTKLWNWVRECLSCDSRCTSDQVETQACTPEQNRVCTCRP
GWYCTLKRQKGCRLCAPLHRCRPGFGVTRPGTATSNVACARKTASTIESK
TGEGPKCPVPEIPGAPSVFIFPPKPKDTLSISRTPEVTCLVVDLGPDDSN
VQITWFVDNTEMHTAKTRPREEQFNSTYRVVSVLPILHQDWLKGKEFKCK
VNSKSLPSAMERTISKAKGQPHEPQVYVLPPTQEELSENKVSVTCLIKGF
HPPDIAVEWEITGQPEPENNYQTTPPQLDSDGTYFLYSRLSVDRSHWQRG
NTYTCSVSHEALHSHHTQKSLTQSPGK

SEQ ID NO:52 (mouse TNFR p80, including the ECD and the transmembrane domain; GenBank Accession Number CAA74969.1)

MAPAALWVAL VFELQLWATG HTVPAQVVLT PYKPEPGYEC
QISQEYYDRK AQMCCAKCPP GQYVKHFCNK TSDTVCADSD
TVCADCEASM YTQVWNQFRT CLSCSSSCST DQVETRACTK
QQNRVCACEA GRYCALKTHS GSCRQCMRLS KCGPGFGVAS
SRAPNGNVLC KACAPGTFSD TTSSTDVCRP HRICSILAIP
GNASTDAVCA PESPTLSAIP RTLYVSQPEP TRSQPLDQEP
GPSQTPSILT SLGSTPIIEQ STKGGISLPI GLIVGVTSLG
LLMLGLVNCF ILVQRKKKPS CLQRDAKVPH VPDEKSQDAV
GLEQQHLLTT APSSSSSSSL ESSASAGDRR APPGGHPQAR
VMAEAQGSQE ARASSRISDS SHGSHGTHVN VTCIVNVCSS
SDHSSQCSSQ ASATVGDPDA KPSASPKDEQ VPFSQEECPS
QSPYETTETL QSHEKPLPLG VPDMGMKPSQ AGWFDQIAVK
VA

SEQ ID NO:53 (mouse TNFR p80, including the ECD and the transmembrane domain; GenBank Accession Numbers P25119.1 or NP_035740.2)

MAPAALWVAL VFELQLWATG HTVPAQVVLT PYKPEPGYEC
QISQEYYDRK AQMCCAKCPP GQYVKHFCNK TSDTVCADCE
ASMYTQVWNQ FRTCLSCSSS CTTDQVEIRA CTKQQNRVCA
CEAGRYCALK THSGSCRQCM RLSKCGPGFG VASSRAPNGN
VLCKACAPGT FSDTTSSTDV CRPHRICSIL AIPGNASTDA
VCAPESPTLS AIPRTLYVSQ PEPTRSQPLD QEPGPSQTPS
ILTSLGSTPI IEQSTKGGIS LPIGLIVGVT SLGLLMLGLV
NCIILVQRKK KPSCLQRDAK VPHVPDEKSQ DAVGLEQQHL
LTTAPSSSSS SLESSASAGD RRAPPGGHPQ ARVMAEAQGF
QEARASSRIS DSSHGSHGTH VNVTCIVNVC SSSDHSSQCS
SQASATVGDP DAKPSASPKD EQVPFSQEEC PSQSPCETTE
TLQSHEKPLP LGVPDMGMKP SQAGWFDQIA VKVA

SEQ ID NO: 54 (the hinge region and the CH2 and CH3 domains of the equine IgG1 heavy chain constant region)

EPIPDNHQKVCDMSKCPKCPAPELLGGPSVFIFPPNPKDTLMITRTPEVT
CVVVDVSQENPDVKFNWYMDGVEVRTATTRPKEEQFNSTYRVVSVLRIQH
QDWLSGKEFKCKVNNQALPQPIERTITKTKGRSQEPQVYVLAPHPDELSK
SKVSVTCLVKDFYPPEINIEWQSNGQPELETKYSTTQAQQDSDGSYFLYS
KLSVDRNRWQQGTTFTCGVMHEALHNHYTQKNVSKNPGK

SEQ ID NO: 55 (the hinge region and the CH2 and CH3 domains of the equine IgG2 heavy chain constant region)

PPCVLSAEGVIPIPSVPKPQCPPYTHSKFLGGPSVFIFPPNPKDALMISR
TPVVTCVVVNLSDQYPDVQFSWYVDNTEVHSAITKQREAQFNSTYRVVSV
LPIQHQDWLSGKEFKCSVTNVGVPQPISRAISRGKGPSRVPQVYVLPPHP
DELAKSKVSVTCLVKDFYPPDISVEWQSNRWPELEGKYSTTPAQLDGDGS
YFLYSKLSLETSRWQQVESFTCAVMHEALHNHFTKTDISESLGK

SEQ ID NO:56 (the hinge region and the CH2 and CH3 domains of the equine IgG3 heavy chain constant region)

EPVLPKPTTPAPTVPLTTTVPVETTTPPCPCECPKCPAPELLGGPSVFIF
PPKPKDVLMITRMPEVTCLVVDVSHDSSDVLFTWYVDGTEVKTAKTMPNE
EQNNSTYRVVSVLRIQHQDWLNGKKFKCKVNNQALPAPVERTISKATGQT
RVPQVYVLAPHPDELSKNKVSVTCLVKDFYPTDITVEWQSNEHPEPEGKY
RTTEAQKDSDGSYFLYSKLTVEKDRWQQGTTFTCVVMHEALHNHVMQKNI
SKNPGK

SEQ ID NO:57 (the hinge region and the CH2 and CH3 domains of the equine IgG4 heavy chain constant region)

VIKECNGGCPAECLQVGPSVFIFPPKPKDVLMISRTPTVTCVVVDVGHDF
PDVQFNWYVDGVETHTATTEPKQEQFNSTYRVVSVLPIQHKDWLSGKEFK
CKVNNKALPAPVERTISKPTGQPREPQVYVLAPHRDELSKNKVSVTCLVK
DFYPTDIDIEWKSNGQPEPETKYSTTPAQLDSDGSYFLYSKLTVETNRWQ
QGTTFTCAVMHEALHNHYTEKSVSKSPGK

SEQ ID NO:58 (the hinge region and the CH2 and CH3 domains of the equine IgG5 heavy chain constant region)

VVKGSPCPKCPAPELPGGPSVFIFPPKPKDVLKISRKPEVTCVVVDLGHD
DPDVQFTWFVDGVETHTATTEPKEEQFNSTYRVVSVLPIQHQDWLSGKEF
KCSVTNKALPAPVERTTSKAKGQLRVPQVYVLAPHPDELAKNTVSVTCLV
KDFYPPEIDVEWQSNEHPEPEGKYSTTPAQLNSDGSYFLYSKLSVETSRW
KQGESFTCGVMHEAVENHYTQKNVSHSPGK

SEQ ID NO:59 (the hinge region and the CH2 and CH3 domains of the equine IgG6 heavy chain constant region)

VIKEPCCCPKCPGRPSVFIFPPNPKDTLMISRTPEVTCVVVDVSQENPDV
KFNWYVDGVEAHTATTKAKEKQDNSTYRWSVLPIQHQDWRRGKEFKCKVN
NRALPAPVERTITKAKGELQDPKVYILAPHREEVTKNTVSVTCLVKDFYP
PDINVEWQSNEEPEPEVKYSTTPAQLDGDGSYFLYSKLTVETDRWEQGES
FTCWMHEAIRHTYROKSITNFPGK

SEQ ID NO:60 (the hinge region and the CH2 and CH3 domains of the equine IgG7 heavy chain constant region)

VIKECGGCPTCPECLSVGPSVFIFPPKPKDVLMISRTPTVTCVVVDVGHD
FPDVQFNWYVDGVETHTATTEPKQEQNNSTYRVVSILAIQHKDWLSGKEF
KCKVNNQALPAPVQKTISKPTGQPREPQVYVLAPHRDELSKNKVSVTCLV
KDFYPTDIDIEWKSNGQPEPETKYSTTPAQLDSDGSYFLYSKLTVETNRW
QQGTTFTCAVMHEALHNHYTEKSVSKSPGK

SEQ ID NO:61 (the hinge region and the CH2 and CH3 domains of the feline IgG1a heavy chain constant region)

RKTDHPPGPKPCDCPKCPPPEMLGGPSIFIFPPKPKDTLSISRTPEVTCL
VVDLGPDDSDVQITWFVDNTQVYTAKTSPREEQFNSTYRVVSVLPILHQD
WLKGKEFKCKVNSKSLPSPIERTISKAKGQPHEPQVYVLPPAQEELSRNK
VSVTCLIKSFHPPDIAVEWEITGQPEPENNYRTTPPQLDSDGTYFVYSKL
SVDRSHWQRGNTYTCSVSHEALHSHHTQKSLTQSPGK

SEQ ID NO:62 (the hinge region and the CH2 and CH3 domains of the feline IgG1b heavy chain constant region)

RKTDHPPGPKPCDCPKCPPPEMLGGPSIFIFPPKPKDTLSISRTPEVTCL
VVDLGPDDSDVQITWFVDNTQVYTAKTSPREEQFNSTYRVVSVLPILHQD
WLKGKEFKCKVNSKSLPSPIERTISKDKGQPHEPQVYVLPPAQEELSRNK
VSVTCLIEGFYPSDIAVEWEITGQPEPENNYRTTPPQLDSDGTYFLYSRL
SVDRSRWQRGNTYTCSVSHEALHSHHTQKSLTQSPGK

SEQ ID NO:63 (the hinge region and the CH2 and CH3 domains of the feline IgG2 heavy chain constant region)

PKTASTIESKTGEGPKCPVPEIPGAPSVFIFPPKPKDTLSISRTPEVTCL
VVDLGPDDSNVQITWFVDNTEMHTAKTRPREEQFNSTYRVVSVLPILHQD
WLKGKEFKCKVNSKSLPSAMERTISKAKGQPHEPQVYVLPPTQEELSENK
VSVTCLIKGFHPPDIAVEWEITGQPEPENNYQTTPPQLDSDGTYFLYSRL
SVDRSHWQRGNTYTCSVSHEALHSHHTQKSLTQSPGK

SEQ ID NO:64 (PREDICTED feline p80 TNFR, TNFRSF1B; XP_003989632.1)

MSNCGHVLAL SGVPPGWVWG CSPGLLIPHV ALLPYVPEPG
SSCQLTEYFD ERTQMCCSKC QPGYHAQSLC SETSNTVCAR
CEDSTYTKLW NWVRECLSCD SRCTSDQVET QACTPEQNRV
CTCRPGWYCT LKRQKGCRLC APLHRCRPGF GVTRPGTATS
NVACAPCGPG TFSDTTSSTD VCRPHRICGS VAIPGNATMD
AVCASVPPTL RMAPRPAPTS QPASTLSQQV EPTPGPRTAP
STSLLFVMVP TPPAEGLSTG DISLLIGLIV AVTSLGLVII
GLVKCVIMTQ KKKKPFCLQG EAKVPHLPAD KARSAPGPEQ
QHLLTTAPSS SSSSLESSGS TADGRAPTGT RLPAPGTEKA
AGSGEAWASS SSSEPSSGSH GTQVNVTCIV NVCSASDHGS
QCASQASYTM GDVDASSSGS PNDEQVPFSK EECPFQSQPG
APETLLENPE EKPLPLGVPD AGMKPS

SEQ ID NO:65 (leader sequence depicted in Genbank accession no. XP_003989632.1)

MSNCGHVLAL SGVPPGWVWG CS

1-39. (canceled)

40. An isolated tumor necrosis factor receptor (TNFR) polypeptide capable of binding to TNF, or a TNF-binding fragment of the polypeptide, wherein the polypeptide comprises an amino acid sequence of SEQ ID NO:1, or an amino acid sequence that has at least 85% identity thereto after optimal alignment, and wherein the polypeptide or the TNF-binding fragment thereof comprises an N-terminal VPAQV motif (SEQ ID NO: 68), with the proviso that the polypeptide is not a mouse TNFR p80.

41. The polypeptide of claim 40, or a TNF-binding fragment thereof, wherein the polypeptide or fragment comprises an N-terminal VPAQVVF motif (SEQ ID NO: 69).

42. The polypeptide of claim 41, or a TNF-binding fragment thereof, wherein the polypeptide comprises an amino acid sequence of SEQ ID NO:2, or an amino acid sequence that has at least 85% identity thereto after optimal alignment.

43. The polypeptide of claim 41, or a TNF-binding fragment thereof, wherein the polypeptide comprises an amino acid sequence of SEQ ID NO:3, or an amino acid sequence that has at least 85% identity thereto after optimal alignment.

44. The polypeptide of claim 40, or a TNF-binding fragment thereof, wherein the polypeptide or fragment comprises an N-terminal VPAQVAL motif (SEQ ID NO: 70).

45. The polypeptide of claim 44, or a TNF-binding fragment thereof, wherein the polypeptide comprises an amino acid sequence of SEQ ID NO:36, or an amino acid sequence that has at least 85% identity thereto after optimal alignment.

46. The polypeptide of claim 44, or a TNF-binding fragment thereof, wherein the polypeptide comprises an amino acid sequence of SEQ ID NO:37, or an amino acid sequence that has at least 85% identity thereto after optimal alignment.

47. A fusion protein comprising a TNFR polypeptide, or a TNF-binding fragment thereof, as claimed in claim 40, linked to a fusion partner.

48. A fusion protein comprising a TNFR polypeptide, or a TNF-binding fragment thereof, as claimed in claim 41, linked to a fusion partner.

49. The fusion protein of claim 47, wherein the fusion partner comprises a CH2 domain and a CH3 domain of an immunoglobulin heavy chain constant region.

50. The fusion protein of claim 48, wherein the fusion partner comprises a CH2 domain and a CH3 domain of an immunoglobulin heavy chain constant region.

51. The fusion protein of claim 48, wherein the fusion partner comprises an amino acid selected from the group consisting of SEQ ID NOs:7-13 and 54-60, or an amino acid sequence that has at least 85% identity to any of SEQ ID NOs:7-13 and 54-60 after optimal alignment.

52. A fusion protein comprising a TNFR polypeptide, or a TNF-binding fragment thereof, as claimed in claim 44, linked to a fusion partner.

53. The fusion protein of claim 52, wherein the fusion partner comprises a CH2 domain and a CH3 domain of an immunoglobulin heavy chain constant region.

54. The fusion protein of claim 52 wherein the fusion partner comprises an amino acid sequence selected from the group consisting of SEQ ID NOs: 43-45 and 61-63, or an amino acid sequence that has at least 85% identity to any of SEQ ID NOs: 43-45 and 61-63 after optimal alignment.

55. An isolated polynucleotide comprising a nucleic acid sequence encoding the fusion protein of claim 48.

56. A pharmaceutical composition comprising the polypeptide of claim 40, or a TNF-binding fragment thereof, and a pharmaceutically acceptable carrier or excipient.

57. A pharmaceutical composition comprising the fusion protein of claim 48 and a pharmaceutically acceptable carrier or excipient.

58. A method for treating or preventing a condition mediated by TNF in an equine, the method comprising the step of administering a therapeutically effective amount of the polypeptide of claim 41, or a TNF-binding fragment thereof, to an equine in need thereof.

59. A method for treating or preventing a condition mediated by TNF in an equine, the method comprising the step of administering a therapeutically effective amount of the fusion protein of claim 48 to an equine in need thereof.

60. A method for treating or preventing a condition mediated by TNF in a feline, the method comprising the step of administering a therapeutically effective amount of the polypeptide of claim 44, or a TNF-binding fragment thereof, to a feline in need thereof.

61. A method for treating or preventing a condition mediated by TNF in a feline, the method comprising the step of administering a therapeutically effective amount of the fusion protein of claim 52 to a feline in need thereof.

62. The method of claim 58, wherein the condition mediated by TNF is selected from the group consisting of an inflammatory mediated condition, a chronic inflammatory disease, arthritis, such as immune mediated polyarthritis, rheumatoid arthritis, osteoarthritis, polyarthritidies, juvenile idiopathic arthritis, psoriatic arthritis, ankylosing spondylitis, Crohn's disease, ulcerative colitis, psoriasis, systemic vasculitis, atopic dermatitis, congestive heart failure, refractory uveitis, bronchial asthma, allergic conditions, sepsis, shock, diabetes mellitus, and neuro-degenerative conditions, such as Alzheimer's disease, Parkinson's disease, stroke and amyotrophic lateral sclerosis.

63. The method of claim 60, wherein the condition mediated by TNF is selected from the group consisting of an inflammatory mediated condition, a chronic inflammatory disease, arthritis, such as immune mediated polyarthritis, rheumatoid arthritis, osteoarthritis, polyarthritidies, juvenile idiopathic arthritis, psoriatic arthritis, ankylosing spondylitis, Crohn's disease, ulcerative colitis, psoriasis, systemic vasculitis, atopic dermatitis, congestive heart failure, refractory uveitis, bronchial asthma, allergic conditions, sepsis, shock, diabetes mellitus, and neuro-degenerative conditions, such as Alzheimer's disease, Parkinson's disease, stroke and amyotrophic lateral sclerosis.


SEQUENCE LISTING (115 sequences)

SEQ ID NO: 1 - protein, 140 aa, Equus caballus
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCA

SEQ ID NO: 2 - protein, 236 aa, Equus caballus
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGN

SEQ ID NO: 3 - protein, 460 aa, Equus caballus
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNFSLPIGLIVGVTALGLLVIGLVNCVIMTQKKKKSFCLQGEAKVPHLPADKAGGAPGQEQQHLLTTAQSSSSSSLESSASTADKRAPTGDQLHAPGTGKASGPGEVRASSSSSAEPSSGSHGTQVNVTCIVNVCSNSGHGSQCPSQTSSTAGDTDASPSDSLKDEQVPFSKEECPCQSQSGASETLLQNPEEKPLPLGVPEGGVKSLTRPARDASQPRGADLWAF

SEQ ID NO: 4 - protein, 22 aa, Equus caballus
MAPVAVWAALAVGLQLWAAGRA

SEQ ID NO: 5 - protein, 482 aa, Equus caballus
MAPVAVWAALAVGLQLWAAGRAVPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNFSLPIGLIVGVTALGLLVIGLVNCVIMTQKKKKSFCLQGEAKVPHLPADKAGGAPGQEQQHLLTTAQSSSSSSLESSASTADKRAPTGDQLHAPGTGKASGPGEVRASSSSSAEPSSGSHGTQVNVTCIVNVCSNSGHGSQCPSQTSSTAGDTDASPSDSLKDEQVPFSKEECPCQSQSGASETLLQNPEEKPLPLGVPEGGVKSLTRPARDASQPRGADLWAF

SEQ ID NO: 6 - DNA, 1449 bp, Equus caballus
atggcgcccgtcgccgtctgggccgcgctggccgtcggactacagctctgggccgcggggcgcgccgtgccggcccaggtggtgtttccccgctctatcccggagcccagcaacttgtgccagccaagagaatactacgacgagagggcccagaggcggtgcagccagtgtccacccggctgccgcgcaaaaagtttctgcaacgagacctcagatacggtgtgtgtcccctgcgaggacagcacatacacccagctctggaactggctccccgagtgcctgagctgtggctcccgctgcagcaccggccaagtggaaactcaagcctgtactttgaagcagaaccgcatctgcacctgcgagccgggcaggtactgcatacttccaaggcaggaggggtgccaggtctgcgggctgctgcgcaagtgcccgcccggcttcggcgtggccaagccaggaactgcgacatcaaacgtggtgtgcgctgcctgtgccccagggacattctccgacaggacgtcgtccacagatacttgcaggccccaccggaactgtagctcggtggccgtccctggcaatgccagcatggatgccgtctgcaagtctgtgcttcccaccccgagagtggcctcggagccagcctctgcgccccagccagggtccacgagatcccatcacgcggagctgacccgcgggcccagcacagctcctggtacctcccccctgcccccgatggttccaagccccccagctgaagggctcatcactggcaacttctcccttccaattgggctgatcgtgggagtgacagccttgggtctgctagtaatcgggctggtgaactgtgtcatcatgacccagaagaaaaagaagtccttctgcctgcaaggagaagccaaggtgcctcacctgcctgctgataaggccggaggggctccgggccaggagcagcagcacctgctgaccacggcgcaaagctccagcagcagctccctggagagctcggccagcaccgcggacaagagggcgcccacgggggaccagctgcatgccccaggcacggggaaggccagcgggcctggggaggtgcgggccagctccagcagctcagcagagccttcctctggcagccacgggacccaggtcaacgtcacatgcatcgtgaatgtttgcagcaattccggccacggctcccagtgcccctcccagaccagctccactgcgggggacacggatgccagcccctcagactccctgaaggacgagcaggtccccttctccaaggaggaatgcccttgtcagtcccagtcgggggcttcagagactctgctgcagaacccagaggagaagccgctgccccttggcgtgcccgagggaggggtgaagtcgttaaccaggccagcaagggacgcgtcacagccgagaggcgctgatctctgggccttctag

SEQ ID NO: 7 - protein, 337 aa, Equus caballus
ASTTAPKVFALAPGCGTTSDSTVALGCLVSGYFPEPVKVSWNSGSLTSGVHTFPSVLQSSGFYSLSSMVTVPASSWTSETYICNVVHAASNFKVDKRIEPIPDNHQKVCDMSKCPKCPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 8 - protein, 342 aa, Equus caballus
ASTTAPKYFQLTPSCGITSDATVALGCLVSDYYPEPVTVSWNSGALTSGVHTFPSVLQSSGLYALSSMVTVPASTWTSETYICNVAHPASSTKVDKRIPPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 9 - protein, 353 aa, Equus caballus
STTAPKVFPLAPSCGNTSDSTVALGCLVSSYFPEPVTVSWNSGTLTSGVRTFPSVLQSSGLYSLSSMVTVPASSLESKTYICNVAHPASSTKVDKRIEPVLPKPTTPAPTVPLTTTVPVETTTPPCPCECPKCPAPELLGGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFYPTDITVEWQSNEHPEPEGKYRTTEAQKDSDGSYFLYSKLTVEKDRWQQGTTFTCVVMHEALHNHVMQKNISKNPGK

SEQ ID NO: 10 - protein, 327 aa, Equus caballus
ASTTAPKVFPLASHSAATSGSTVALGCLVSSYFPEPVTVSWNSGALTSGVHTFPSVLQSSGLYSLSSMVTVPASSLKSQTYICNVAHPASSTKVDKKIVIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 11 - protein, 326 aa, Equus caballus
ESPKAPDVFPLTICGNTPDPTVPVGCLVSNYFPEPVTVSWNCDALKGDIHTFPLDLSNSAHHSLSSMMAVPRSSLNQTYICSVAHPASSTKVDKRIVVKGSPCPKCPAPELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 12 - protein, 322 aa, Equus caballus
ASTTAPKVFQLASHSAGTSDSTVALGCLVSSYFPEPVTVSWNSGALTSGVHTFPSVRQSSGLYSLSSMVTVPASSLKSQTYICNVAHPASSTKVDKRIVIKEPCCCPKCPGRPSVFIFPPNPKDTLMISRTPEVTCVVVDVSQENPDVKFNWYVDGVEAHTATTKAKEKQDNSTYRWSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCWMHEAIRHTYRQKSITNFPGK

SEQ ID NO: 13 - protein, 328 aa, Equus caballus
ASTTAPKVFPLASHSAATSGSTVALGCLVSSYFPEPVTVSWNSGALTSGVHTFPSVLQSSGLYSLSSMVTVPASSLKSQTYICNVAHPASSTKVDKKIVIKECGGCPTCPECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 14 - protein, 578 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNASTTAPKYEQLTPSCGITSDATVALGCLVSDYYPEPVTVSWNSGALTSGVHTFPSVLQSSGLYALSSMVTVPASTWTSETYICNVAHPASSTKVDKRIPPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 15 - protein, 558 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNASTTAPKVFQLASHSAGTSDSTVALGCLVSSYFPEPVTVSWNSGALTSGVHTFPSVRQSSGLYSLSSMVTVPASSLKSQTYICNVAHPASSTKVDKRIVIKEPCCCPKCPGRPSVFIFPPNPKDTLMISRTPEVTCVVVDVSQENPDVKFNWYVDGVEAHTATTKAKEKQDNSTYRWSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCWMHEAIRHTYRQKSITNFPGK

SEQ ID NO: 16 - protein, 600 aa, Artificial Sequence
  Description of Artificial Sequence Synthetic polypeptide
MAPVAVWAALAVGLQLWAAGRAVPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNASTTAPKYEQLTPSCGITSDATVALGCLVSDYYPEPVTVSWNSGALTSGVHTFPSVLQSSGLYALSSMVTVPASTWTSETYICNVAHPASSTKVDKRIPPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 17 - protein, 580 aa, Artificial Sequence
  Description of Artificial Sequence Synthetic polypeptide
MAPVAVWAALAVGLQLWAAGRAVPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNASTTAPKVFQLASHSAGTSDSTVALGCLVSSYFPEPVTVSWNSGALTSGVHTFPSVRQSSGLYSLSSMVTVPASSLKSQTYICNVAHPASSTKVDKRIVIKEPCCCPKCPGRPSVFIFPPNPKDTLMISRTPEVTCVVVDVSQENPDVKFNWYVDGVEAHTATTKAKEKQDNSTYRWSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCWMHEAIRHTYRQKSITNFPGK

SEQ ID NO: 18 - protein, 32 aa, Equus caballus
MGEEAGVEGARASPFYSYLLHVEKSPITFPLQ

SEQ ID NO: 19 - protein, 488 aa, Equus caballus
MGEEAGVEGARASPFYSYLLHVEKSPITFPLQVVFPRSVPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNFSLPIGLIVGVTALGLLVIGLVNCVIMTQKKKKSFCLQGEAKVPHLPADKAGGAPGQEQQHLLTTAQSSSSSSLESSASTADKRAPTGDQLHAPGTGKASGPGEVRASSSSSAEPSSGSHGTQVNVTCIVNVCSNSGHGSQCPSQTSSTAGDTDASPSDSLKDEQVPFSKEECPCQSQSGASETLLQNPEEKPLPLGVPEGGVKSLTRPARDASQPRGADLWAF

SEQ ID NO: 20 - protein, 497 aa, Artificial Sequence
  Description of Artificial Sequence Synthetic polypeptide
MAPVAVWAALAVGLQLWAAGRAVPAQVVFPRSVPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNEPIPDNHQKVCDMSKCPKCPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 21 - protein, 475 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNEPIPDNHQKVCDMSKCPKCPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 22 - protein, 480 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNPPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 23 - protein, 492 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNEPVLPKPTTPAPTVPLTTTVPVETTTPPCPCECPKCPAPELLGGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFYPTDITVEWQSNEHPEPEGKYRTTEAQKDSDGSYFLYSKLTVEKDRWQQGTTFTCVVMHEALHNHVMQKNISKNPGK

SEQ ID NO: 24 - protein, 465 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNVIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 25 - protein, 466 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNVVKGSPCPKCPAPELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 26 - protein, 460 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNVIKEPCCCPKCPGRPSVFIFPPNPKDTLMISRTPEVTCVVVDVSQENPDVKFNWYVDGVEAHTATTKAKEKQDNSTYRWSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCWMHEAIRHTYRQKSITNFPGK

SEQ ID NO: 27 - protein, 466 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAACAPGTFSDRTSSTDTCRPHRNCSSVAVPGNASMDAVCKSVLPTPRVASEPASAPQPGSTRSHHAELTRGPSTAPGTSPLPPMVPSPPAEGLITGNVIKECGGCPTCPECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 28 - protein, 379 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAEPIPDNHQKVCDMSKCPKCPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 29 - protein, 384 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAPPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 30 - protein, 396 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAEPVLPKPTTPAPTVPLTTTVPVETTTPPCPCECPKCPAPELLGGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFYPTDITVEWQSNEHPEPEGKYRTTEAQKDSDGSYFLYSKLTVEKDRWQQGTTFTCVVMHEALHNHVMQKNISKNPGK

SEQ ID NO: 31 - protein, 369 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAVIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 32 - protein, 370 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAVVKGSPCPKCPAPELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 33 - protein, 364 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAVIKEPCCCPKCPGRPSVFIFPPNPKDTLMISRTPEVTCVVVDVSQENPDVKFNWYVDGVEAHTATTKAKEKQDNSTYRWSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCWMHEAIRHTYRQKSITNFPGK

SEQ ID NO: 34 - protein, 370 aa, Artificial Sequence
  Description of Artificial Sequence Synthetic polypeptide
VPAQVVFPRSIPEPSNLCQPREYYDERAQRRCSQCPPGCRAKSFCNETSDTVCVPCEDSTYTQLWNWLPECLSCGSRCSTGQVETQACTLKQNRICTCEPGRYCILPRQEGCQVCGLLRKCPPGFGVAKPGTATSNVVCAVIKECGGCPTCPECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 35 - protein, 20 aa, Equus caballus
EPIPDNHQKVCDMSKCPKCP

SEQ ID NO: 36 - protein, 86 aa, Felis catus
RCEDSTYTKLWNWVRECLSCDSRCTSDQVETQACTPEQNRVCTCRPGWYCTLKRQKGCRLCAPLHRCRPGFGVTRPGTATSNVACA

SEQ ID NO: 37 - protein, 236 aa, Felis catus
VPAQVALLPYVPEPGSSCQLTEYFDERTQMCCSKCQPGYHAQSLCSETSNTVCARCEDSTYTKLWNWVRECLSCDSRCTSDQVETQACTPEQNRVCTCRPGWYCTLKRQKGCRLCAPLHRCRPGFGVTRPGTATSNVACAPCGPGTFSDTTSSTDVCRPHRICGSVAIPGNATMDAVCASVPPTLRMAPRPAPTSQPASTLSQQVEPTPGPRTAPSTSLLFVMVPTPPAEGLSTGD

SEQ ID NO: 38 - protein, 22 aa, Felis catus
MAPVAVWATLAVGLQLWAAGRA

SEQ ID NO: 39 - protein, 22 aa, Felis catus
MAPAALWATLAVGLQLWAAGRA

SEQ ID NO: 40 - protein, 473 aa, Artificial Sequence
  Description of Artificial Sequence Synthetic polypeptide
VPAQVALLPYVPEPGSSCQLTEYFDERTQMCCSKCQPGYHAQSLCSETSNTVCARCEDSTYTKLWNWVRECLSCDSRCTSDQVETQACTPEQNRVCTCRPGWYCTLKRQKGCRLCAPLHRCRPGFGVTRPGTATSNVACAPCGPGTFSDTTSSTDVCRPHRICGSVAIPGNATMDAVCASVPPTLRMAPRPAPTSQPASTLSQQVEPTPGPRTAPSTSLLFVMVPTPPAEGLSTGDRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 41 - protein, 495 aa, Artificial Sequence
  Description of Artificial Sequence Synthetic polypeptide
MAPVAVWATLAVGLQLWAAGRAVPAQVALLPYVPEPGSSCQLTEYFDERTQMCCSKCQPGYHAQSLCSETSNTVCARCEDSTYTKLWNWVRECLSCDSRCTSDQVETQACTPEQNRVCTCRPGWYCTLKRQKGCRLCAPLHRCRPGFGVTRPGTATSNVACAPCGPGTFSDTTSSTDVCRPHRICGSVAIPGNATMDAVCASVPPTLRMAPRPAPTSQPASTLSQQVEPTPGPRTAPSTSLLFVMVPTPPAEGLSTGDRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 42 - DNA, 1392 bp, Felis catus
atggcgcccgtcgccgtgtgggccacgctggccgtcggactgcagctctgggccgcggggcgcgccgtgcccgcccaggttgcgttactcccctacgttccggagcctggaagctcatgccaactgacagaatacttcgatgagaggacccagatgtgctgtagcaagtgtcagcctggctaccatgcacagtcgctgtgctccgagacctcaaataccgtgtgtgcccgctgcgaggacagcacctacaccaagctctggaactgggtgcgcgagtgcttgagctgtgactcccgctgcacctctgaccaagtggagactcaggcctgcactccggaacagaaccgcgtctgcacctgcaggccgggctggtactgcacgttgaagaggcagaaagggtgccggctgtgtgcgcccctgcacaggtgccgcccgggcttcggcgtgaccaggccaggaactgcaacgtcaaatgtggcgtgcgctccctgtggcccagggacgttctccgacacaacgtcgtccacagatgtctgcaggccccaccggatctgtggctcagtggccatccctggcaatgcaaccatggatgccgtctgcgcatctgtgcctcctaccctgaggatggccccacgcccagcccccacgtcccagccagcgtctacactatcccagcaagtggagccgactccaggccctcgcacggcccctagcacctcccttctgtttgtgatggtcccaacccccccagctgaagggctcagcacgggcgacatctctctcctcattggactgattgtggctgtgacatccttgggtctggtgatcatagggctggtgaaatgtgtcattatgacccagaaaaaaaagaagcccttctgtctacaaggagaagccaaagtgcctcacctgcctgctgacaaggcccgaagtgcccccggccccgagcagcagcacctgctgaccacagcgcccagctccagcagcagctccctggagagctcaggcagcaccgcagacgggagggcgcccaccgggacccggctgcccgcaccaggcacggagaaggccgccgggtctggggaggcctgggccagctccagcagctcagagccttcctctggcagccacgggacccaggtcaacgtcacatgcatcgtgaatgtgtgcagcgcctccgaccacggctctcagtgcgcctcccaggccagctacacgatgggggacgtggatgccagctcctccggctccccaaatgacgagcaggtccccttctccaaggaagaatgcccttttcagtctcagccaggggccccggagactctgctggagaacccggaggagaagcccctgccccttggtgtgcctgatgctgggatgaagcccagttag

SEQ ID NO: 43 - protein, 335 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 44 - protein, 335 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 45 - protein, 335 aa, Felis catus
ASTTASSVFPLAPSCGTTSGATVALACLVLGYFPEPVTVSWNSGALTSGVHTFPSVLQASGLYSLSSMVTVPSSRWLSDTFTCNVAHRPSSTKVDKTVPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 46 - protein, 473 aa, Artificial Sequence; identical to SEQ ID NO: 40
  Description of Artificial Sequence Synthetic polypeptide

SEQ ID NO: 47 - protein, 473 aa, Artificial Sequence
  Description of Artificial Sequence Synthetic polypeptide
VPAQVALLPYVPEPGSSCQLTEYFDERTQMCCSKCQPGYHAQSLCSETSNTVCARCEDSTYTKLWNWVRECLSCDSRCTSDQVETQACTPEQNRVCTCRPGWYCTLKRQKGCRLCAPLHRCRPGFGVTRPGTATSNVACAPCGPGTFSDTTSSTDVCRPHRICGSVAIPGNATMDAVCASVPPTLRMAPRPAPTSQPASTLSQQVEPTPGPRTAPSTSLLFVMVPTPPAEGLSTGDRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 48 - protein, 473 aa, Artificial Sequence
  Description of Artificial Sequence Synthetic polypeptide
VPAQVALLPYVPEPGSSCQLTEYFDERTQMCCSKCQPGYHAQSLCSETSNTVCARCEDSTYTKLWNWVRECLSCDSRCTSDQVETQACTPEQNRVCTCRPGWYCTLKRQKGCRLCAPLHRCRPGFGVTRPGTATSNVACAPCGPGTFSDTTSSTDVCRPHRICGSVAIPGNATMDAVCASVPPTLRMAPRPAPTSQPASTLSQQVEPTPGPRTAPSTSLLFVMVPTPPAEGLSTGDPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 49 - protein, 377 aa, Artificial Sequence
  Description of Artificial Sequence Synthetic polypeptide
VPAQVALLPYVPEPGSSCQLTEYFDERTQMCCSKCQPGYHAQSLCSETSNTVCARCEDSTYTKLWNWVRECLSCDSRCTSDQVETQACTPEQNRVCTCRPGWYCTLKRQKGCRLCAPLHRCRPGFGVTRPGTATSNVACARKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 50 - protein, 377 aa, Artificial Sequence
  Description of Artificial Sequence Synthetic polypeptide
VPAQVALLPYVPEPGSSCQLTEYFDERTQMCCSKCQPGYHAQSLCSETSNTVCARCEDSTYTKLWNWVRECLSCDSRCTSDQVETQACTPEQNRVCTCRPGWYCTLKRQKGCRLCAPLHRCRPGFGVTRPGTATSNVACARKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 51 - protein, 377 aa, Artificial Sequence
  Description of Artificial Sequence Synthetic polypeptide
VPAQVALLPYVPEPGSSCQLTEYFDERTQMCCSKCQPGYHAQSLCSETSNTVCARCEDSTYTKLWNWVRECLSCDSRCTSDQVETQACTPEQNRVCTCRPGWYCTLKRQKGCRLCAPLHRCRPGFGVTRPGTATSNVACAPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 52 - protein, 482 aa, Mus musculus
MAPAALWVALVFELQLWATGHTVPAQVVLTPYKPEPGYECQISQEYYDRKAQMCCAKCPPGQYVKHFCNKTSDTVCADSDTVCADCEASMYTQVWNQFRTCLSCSSSCSTDQVETRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCFILVQRKKKPSCLQRDAKVPHVPDEKSQDAVGLEQQHLLTTAPSSSSSSSLESSASAGDRRAPPGGHPQARVMAEAQGSQEARASSRISDSSHGSHGTHVNVTCIVNVCSSSDHSSQCSSQASATVGDPDAKPSASPKDEQVPFSQEECPSQSPYETTETLQSHEKPLPLGVPDMGMKPSQAGWFDQIAVKVA

SEQ ID NO: 53 - protein, 474 aa, Mus musculus
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 54 - protein, 239 aa, Equus caballus
EPIPDNHQKVCDMSKCPKCPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 55 - protein, 244 aa, Equus caballus
PPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 56 - protein, 256 aa, Equus caballus
EPVLPKPTTPAPTVPLTTTVPVETTTPPCPCECPKCPAPELLGGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFYPTDITVEWQSNEHPEPEGKYRTTEAQKDSDGSYFLYSKLTVEKDRWQQGTTFTCVVMHEALHNHVMQKNISKNPGK

SEQ ID NO: 57 - protein, 229 aa, Equus caballus
VIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 58 - protein, 230 aa, Equus caballus
VVKGSPCPKCPAPELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 59 - protein, 224 aa, Equus caballus
VIKEPCCCPKCPGRPSVFIFPPNPKDTLMISRTPEVTCVVVDVSQENPDVKFNWYVDGVEAHTATTKAKEKQDNSTYRWSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCWMHEAIRHTYRQKSITNFPGK

SEQ ID NO: 60 - protein, 230 aa, Equus caballus
VIKECGGCPTCPECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 61 - protein, 237 aa, Felis catus
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 62 - protein, 237 aa, Felis catus
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 63 - protein, 237 aa, Felis catus
PKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 64 - protein, 466 aa, Felis catus
MSNCGHVLALSGVPPGWVWGCSPGLLIPHVALLPYVPEPGSSCQLTEYFDERTQMCCSKCQPGYHAQSLCSETSNTVCARCEDSTYTKLWNWVRECLSCDSRCTSDQVETQACTPEQNRVCTCRPGWYCTLKRQKGCRLCAPLHRCRPGFGVTRPGTATSNVACAPCGPGTFSDTTSSTDVCRPHRICGSVAIPGNATMDAVCASVPPTLRMAPRPAPTSQPASTLSQQVEPTPGPRTAPSTSLLFVMVPTPPAEGLSTGDISLLIGLIVAVTSLGLVIIGLVKCVIMTQKKKKPFCLQGEAKVPHLPADKARSAPGPEQQHLLTTAPSSSSSSLESSGSTADGRAPTGTRLPAPGTEKAAGSGEAWASSSSSEPSSGSHGTQVNVTCIVNVCSASDHGSQCASQASYTMGDVDASSSGSPNDEQVPFSKEECPFQSQPGAPETLLENPEEKPLPLGVPDAGMKPS

SEQ ID NO: 65 - protein, 22 aa, Felis catus
MSNCGHVLALSGVPPGWVWGCS

SEQ ID NO: 66 - protein, 32 aa, Artificial Sequence; identical to SEQ ID NO: 18
  Description of Artificial Sequence Synthetic polypeptide

SEQ ID NO: 67 - DNA, 3076 bp, Equus caballus
atgggggaggaggcgggggtggagggagccagggcctctcccttctactcctacctgctccacgtggagaagtccccaatcaccttcccgctccaggtggtgtttccccgctctgtcccggagcccagcaacttgtgccagccaagagaatactacgacgagagggcccagaggcggtgcagccagtgtccacccggctgccgcgcaaaaagtttctgcaacgagacctcagatacggtgtgtgtcccctgcgaggacagcacatacacccagctctggaactggctccccgagtgcctgagctgtggctcccgctgcagcaccggccaagtggaaactcaagcctgtactttgaagcagaaccgcatctgcacctgcgagccgggcaggtactgcatacttccaaggcaggaggggtgccaggtctgcgggctgctgcgcaagtgcccgcccggcttcggcgtggccaagccaggaactgcgacatcaaacgtggtgtgcgctgcctgtgccccagggacattctccgacaggacgtcgtccacagatacttgcaggccccaccggaactgtagctcggtggccgtccctggcaatgccagcatggatgccgtctgcaagtctgtgcttcccaccccgagagtggcctcggagccagcctctgcgccccagccagggtccacgagatcccatcacgcggagctgacccgcgggcccagcacagctcctggtacctcccccctgcccccgatggttccaagccccccagctgaagggctcatcactggcaacttctcccttccaattgggctgatcgtgggagtgacagccttgggtctgctagtaatcgggctggtgaactgtgtcatcatgacccagaagaaaaagaagtccttctgcctgcaaggagaagccaaggtgcctcacctgcctgctgataaggccggaggggctccgggccaggagcagcagcacctgctgaccacggcgcaaagctccagcagcagctccctggagagctcggccagcaccgcggacaagagggcgcccacgggggaccagctgcatgccccaggcacggggaaggccagcgggcctggggaggtgcgggccagctccagcagctcagcagagccttcctctggcagccacgggacccaggtcaacgtcacatgcatcgtgaatgtttgcagcaattccggccacggctcccagtgcccctcccagaccagctccactgcgggggacacggatgccagcccctcagactccctgaaggacgagcaggtccccttctccaaggaggaatgcccttgtcagtcccagtcgggggcttcagagactctgctgcagaacccagaggagaagccgctgccccttggcgtgcccgagggaggggtgaagtcgttaaccaggccagcaagggacgcgtcacagccgagaggcgctgatctctgggccttctaggcccccagccccaggactctggctctttctgggccaaattcctttagcggcctccagcctcagccttcctctcccctgcaggcagagggacgaggcagggagagtcgtggaaagctctcccgtcatggtgcgcgtccctcccagaaggctagctgggcatggacctgctgggcagcccggggcgagccctgcctctctgagagctgcccccagcctgtcctgcgagcttggcttctgcagcccttgttgagatcttcctccctgggcctgctggggtggaggggtgccgtgtgcgccatctgtggagcagggcagtggaggaagggcagcgccttagcctggatgctgggactgctggccggtcccagcgggggtggcggtgtcatccaccagatggttctgtgccgggtggggctggcagtccctgtagggcggtcccccaggttcctcaggacccttgaaaagcaccacctccaatccgttcctgccagctccacctcttgtacagtggtgtgaaaaccagctgcgcagaggcccaggcaggcaagtggaatgagggccccggccggggcaagatggcaaaccactaaccgatagtcttccacttttaacacgtggcaattaaaaagaaaaaggaagccccatgcaggcaaccaagccaaccacataacaaagccaaactcaagagtttgtgtcctctgcctttgaccttcgtttcaggtccccccagctccggcccctgctccctgctgtgtggggtgggttctcccatcagggagtttcaggaactggagatgactgaggcccgcagccatctctctcctcctacctcagcctggaactttctcctttgcctcccgagaggctgctcctgttttccctgaccccctcctgctgttcctggaggcccagccccaggggagccatggggccgtgttggtactgtgtccgctgcgatcccaagtggatgtgagatgccgagagcctgtcttgtgtgtgtgtgtctgtgtcacgttgtgggcctgtgtagccatgttgtcgggttgaatagcctgcctgaagcagctgaagctgacaattctgccaaaggattccttcattaaagaggcacccctctccccccacacacacccccgagaccctgcagagggtggacgaatgtggccttgaccagattctgaaggaaggtgggtggggactccttgagaaggcagttgggtcccaagcacttggatcccttgttgaaaaattccactggacttgaacttggcagctgaacctttggagggtgggagagtccagccattacaacggaggcgggaacggcttccaccctggaatggggatatcagctgcctggactggctggggtctcggcccacgttggaccctgcagggcgatggggcatgtctgccccctggtggacagtcctgggaggacctcggccgcactgtgatctccctgcagagccaggaagctggagatcggaaggagaccctttctgggtgtggcctcagttgcccagctggtgttgatgcccagacaatgagtttgcactgttgctggacagcatccttgcttatcaataaaccttttgtttgttgtaca

SEQ ID NO: 68 - protein, 5 aa, Artificial Sequence
  Description of Artificial Sequence Synthetic peptide
VPAQV

SEQ ID NO: 69 - protein, 7 aa, Artificial Sequence
  Description of Artificial Sequence Synthetic peptide
VPAQVVF

SEQ ID NO: 70 - protein, 7 aa, Artificial Sequence
  Description of Artificial Sequence Synthetic peptide
VPAQVAL

SEQ ID NO: 71 - protein, 7 aa, Artificial Sequence
  Description of Artificial Sequence Synthetic peptide
VPAQVVL

SEQ ID NO: 72 - protein, 6 aa, Artificial Sequence
  Description of Artificial Sequence Synthetic peptide
VPAQVV

SEQ ID NO: 73 - protein, 6 aa, Artificial Sequence
  Description of Artificial Sequence Synthetic peptide
VPAQVA

SEQ ID NO: 74 - protein, 7 aa, Artificial Sequence
  Description of Artificial Sequence Synthetic peptide
VPAQVAF

SEQ ID NO: 75 - protein, 32 aa, Equus caballus; identical to SEQ ID NO: 18

SEQ ID NO: 76 - protein, 7 aa, Artificial Sequence
  Description of Artificial Sequence Synthetic peptide
  MOD_RES (4)..(4): Ala or Val
  MOD_RES (6)..(6): Leu or Val
MAPXAXW

SEQ ID NO: 77 - DNA, 91 bp, Canus sp.
atggcgcccgccgccctgtgggccctgctggccgccggactgcagctctggggcgcggggcgcgccgtgcccggccaggccacgcagctcc

SEQ ID NO: 78 - DNA, 91 bp, Homo sapiens
atggcgcccgtcgccgtctgggccgcgctggccgtcggactggagctctgggctgcggcgcacgccttgcccgcccaggtggcatttacac

SEQ ID NO: 79 - DNA, 91 bp, Mus musculus
atggcgcccgccgccctctgggtcgcgctggtcttcgaactgcagctgtgggccaccgggcacacagtgcccgcccaggttgtcttgacac

SEQ ID NO: 80 - DNA, 91 bp, Sus sp.
atggcgcccgccgccgtctgggccgcgctgaccgtcggactgcagctctgggccgctgggcgcgccgtgccctcccaggctgtgttcatgc

SEQ ID NO: 81 - DNA, 20 bp, Artificial Sequence
  Description of Artificial Sequence Synthetic oligonucleotide
atggcgcccgycgccststg

SEQ ID NO: 82 - DNA, 189 bp, Equus caballus
  CDS (1)..(189)
gtggtgtttccccgctctgtcccggagcccagcaacttgtgccagccaagagaatactacgacgagagggcccagaggcggtgcagccagtgtccacccggctgccgcgcaaaaagtttctgcaacgagacctcagatacggtgtgtgtcccctgcgaggacagcacatacacccagctctggaactgg

SEQ ID NO: 83 - protein, 63 aa, Equus caballus
VVFPRSVPEPSNLCQPREYYDERAQRRCSQCPPGCRAKSFCNETSDTVCVPCEDSTYTQLWNW

SEQ ID NO: 84 - protein, 28 aa, Artificial Sequence
  Description of Artificial Sequence Synthetic peptide
MAPVAVWAALAVGLQLWAAGRAVPAQVV

SEQ ID NO: 85 - protein, 28 aa, Canus sp.
MAPAALWALLAAGLQLWGAGRAVPGQAT

SEQ ID NO: 86 - protein, 28 aa, Mus musculus
MAPAALWVALVFELQLWATGHTVPAQVV

SEQ ID NO: 87 - protein, 28 aa, Homo sapiens
MAPVAVWAALAVGLELWAAAHALPAQVA

SEQ ID NO: 88 - protein, 28 aa, Sus sp.
MAPAAVWAALTVGLQLWAAGRAVPSQAV

SEQ ID NO: 89 - protein, 70 aa, Artificial Sequence
  Description of Artificial Sequence Synthetic polypeptide
MAPVAVWAALAVGLQLWAAGRAVPAQVVFPRSIPEPSNLCQPREYYDERAQRRCSQCPPGCRAKSFCNET

SEQ ID NO: 90 - protein, 90 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWV

SEQ ID NO: 91 - protein, 91 aa, Mus musculus
MAPAALWVALVFELQLWATGHTVPAQVVLTPYKPEPGYECQISQEYYDRKAQMCCAKCPPGQYVKHFCNKTSDTVCADCEASMYTQVWNQF

SEQ ID NO: 92 - protein, 92 aa, Canus sp.
MAPAALWALLAAGLQLWGAGRAVPGQATQLPYVPDPELGSSCQQSEYFDQRTQMCCSMCPPGSHARLFCTKTSNTVCARCENSTYTQLWNWV

SEQ ID NO: 93 - protein, 92 aa, Sus sp.
MAPAAVWAALTVGLQLWAAGRAVPSQAVFMPYAPELGSSCRLPLKEYYDTKAQMCCSKCPPGFRIQTSCNRTSDTVCGSCESSTYTQLWNSV

SEQ ID NO: 94 - protein, 93 aa, Felis catus
MSNCGHVLALSGVPPGWVWGCSPGLLIPHVALLPYVPEPGSSCQLTEYFDERTQMCCSKCQPGYHAQSLCSETSNTVCARCEDSTYTKLWNWV

SEQ ID NO: 95 - protein, 96 aa, Equus caballus
MGEEAGVEGARASPFYSYLLHVEKSPITFPLQVVFPRSVPEPSNLCQPREYYDERAQRRCSQCPPGCRAKSFCNETSDTVCVPCEDSTYTQLWNWL

SEQ ID NO: 96 - protein, 70 aa, Equus caballus
MGEEAGVEGARASPFYSYLLHVEKSPITFPLQVVFPRSVPEPSNLCQPREYYDERAQRRCSQCPPGCRAK

SEQ ID NO: 97 - protein, 70 aa, Homo sapiens
MAPVAVWAALAVGLELWAAAHALPAQVAFTPYAPEPGSTCRLREYYDQTAQMCCSKCSPGQHAKVFCTKT

SEQ ID NO: 98 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 99 - protein, 454 aa, Equus caballus
MGLPAVPGLLPPLALLALLVEIYPSGVTGLVPPLRDREKRDSLCPQGKYSHPQNNSICCTKCHKGTYLHNDCLGPGLDTDCRECENGTFTASENHLRQCLSCSKCRKEMNQVEISSCTVDRDTVCGCRKNQYRKYWSETSETRFQCLDCSLCINGTVHLPCQERQNTVCTWPHGLFWKENECMSCVKCKKDAECTKLCPSPTQSVTGRQDPGATVLLPLVIFLGLCLLSLLSIGFLCRYRRWKPKLRSIVCGKSTPVKEGEPEPLAPTPSFSPTAALSVIPGYSPVSSSTFTPIPNFTPSDWRNFSAASPPREMAPPYQEAGPILTAVPPSSPLSTPVQKWEDSAHTQRPDSDRPGTLYAVVDGVPPSRWKEFMRRLGLSEHEIERLELQNGRCLREAHYSMLAAWRRRTPRREATLELLGSVLRDMDLLGCLEDIQEKLCAPASHSPAPRLPR

SEQ ID NO: 100 - DNA, 1449 bp, Equus caballus; identical to SEQ ID NO: 6

SEQ ID NO: 101 - protein, 59 aa, Canus sp.
MAPAALWALLAAGLQLWGAGRAVPGQATQLPYVPDPELGSSCQQSEYFDQRTQMCCSMC

SEQ ID NO: 102 - protein, 57 aa, Equus caballus
MAPVAVWAALAVGLQLWAAGRAVPAQVVFPRSIPEPSNLCQPREYYDERAQRRCSQC

SEQ ID NO: 103 - protein, 58 aa, Mus musculus
MAPAALWVALVFELQLWATGHTVPAQVVLTPYKPEPGYECQISQEYYDRKAQMCCAKC

SEQ ID NO: 104 - protein, 57 aa, Homo sapiens
MAPVAVWAALAVGLELWAAAHALPAQVAFTPYAPEPGSTCRLREYYDQTAQMCCSKC

SEQ ID NO: 105 - protein, 59 aa, Sus sp.
MAPAAVWAALTVGLQLWAAGRAVPSQAVFMPYAPELGSSCRLPLKEYYDTKAQMCCSKC

SEQ ID NO: 106 - DNA, 300 bp, Felis catus
  CDS (1)..(300)
atgagcaactgtgggcatgtgttggcactcagcggtgtgccgccagggtgggtctggggctgctccccggggctgctcatcccccatgttgcgttactcccctacgttccggagcctggaagctcatgccaactgacagaatacttcgatgagaggacccagatgtgctgtagcaagtgtcagcctggctaccatgcacagtcgctgtgctccgagacctcaaataccgtgtgtgcccgctgcgaggacagcacctacaccaagctctggaactgggtgcgcgagtgcttgagctgtgac

SEQ ID NO: 107 - protein, 100 aa, Felis catus
MSNCGHVLALSGVPPGWVWGCSPGLLIPHVALLPYVPEPGSSCQLTEYFDERTQMCCSKCQPGYHAQSLCSETSNTVCARCEDSTYTKLWNWVRECLSCD

SEQ ID NO: 108 - protein, 58 aa, Artificial Sequence
  Description of Artificial Sequence Synthetic polypeptide
MAPVAVWATLAVGLQLWAAGRAVPAQVALLPYVPEPGSSCQLTEYFDERTQMCCSKCQ

SEQ ID NO: 109 - protein, 58 aa, Artificial Sequence
  Description of Artificial Sequence Synthetic polypeptide
MAPAALWATLAVGLQLWAAGRAVPAQVALLPYVPEPGSSCQLTEYFDERTQMCCSKCQ

SEQ ID NO: 110 - protein, 60 aa, Canus sp.
MAPAALWALLAAGLQLWGAGRAVPGQATQLPYVPDPELGSSCQQSEYFDQRTQMCCSMCP

SEQ ID NO: 111 - protein, 58 aa, Equus caballus
MAPVAVWAALAVGLQLWAAGRAVPAQVVFPRSIPEPSNLCQPREYYDERAQRRCSQCP

SEQ ID NO: 112 - protein, 59 aa, Mus musculus
MAPAALWVALVFELQLWATGHTVPAQVVLTPYKPEPGYECQISQEYYDRKAQMCCAKCP

SEQ ID NO: 113 - protein, 58 aa, Homo sapiens
MAPVAVWAALAVGLELWAAAHALPAQVAFTPYAPEPGSTCRLREYYDQTAQMCCSKCS

SEQ ID NO: 114 - protein, 60 aa, Sus sp.
MAPAAVWAALTVGLQLWAAGRAVPSQAVFMPYAPELGSSCRLPLKEYYDTKAQMCCSKCP

SEQ ID NO: 115 - protein, 70 aa, Felis catus
MAPAALWATLAVGLQLWAAGRAYPAQYALLPYYPEPGSSCQLTEYFDERTQWCCSKCQPGYHAQSLCSET